(19) United States
(12) Patent Application Publication
(10) Pub. No.: US 2026/0224511 A1
(43) Pub. Date: Aug. 6, 2026

(54) PLASMALOGEN MODULATION OF IMMUNE CELLS

(71) Applicant: BAKER HEART AND DIABETES INSTITUTE, Melbourne (AU)

(72) Inventors: Graeme LANCASTER, Melbourne (AU); Peter John MEIKLE, South Yarra (AU); Sudip PAUL, Truganina (AU); Kevin HUYNH, Melbourne (AU); Andrew MURPHY, Melbourne (AU); Pooranee K. Chanthira MORGAN, Melbourne (AU)

(73) Assignee: BAKER HEART AND DIABETES INSTITUTE, Melbourne (AU)

(21) Appl. No.: 19/155,131

(22) PCT Filed: Feb. 9, 2024
(86) PCT No.: PCT/EP2024/053394

§ 371 Date: Aug. 8, 2025

(30) Foreign Application Priority Data
Feb. 10, 2023 (AU) 2023900331

(51) Int. Cl. A61K 31/08; A61K 31/22; A61K 31/685; A61P 37/04

(52) U.S. Cl. CPC A61K 31/08; A61K 31/22; A61K 31/685; A61P 37/04

(57) ABSTRACT

The disclosure relates to certain compounds that can be consumed by a subject to modulate and/or increase mixtures of ether lipid molecules, such as plasmalogens, in immune cells of the subject. The disclosure further relates to compositions and/or formulations comprising said compounds, compositions and/or formulations comprising at least two compounds, and uses thereof.

This application claims priority to Australian Provisional Patent Application No. AU2023900331, filed on 10 Feb. 2023, the entire contents of which is herein incorporated by reference in its entirety.

### FIELD

This disclosure generally relates to compositions and methods for modulating and increasing mixtures of ether lipid molecules, such as plasmalogens, in immune cells of a human subject.

### BACKGROUND

The reference in this specification to any prior publication, or to any matter which is known, is not, and should not be taken as an acknowledgment or admission or any form of suggestion that that prior publication or known matter forms part of the common general knowledge in the field of endeavour to which this specification relates.

Bibliographic details of documents referred to are listed at the end of the specification.

Poor immune health and associated diseases, disorders or conditions, such as metabolic disease, encompassing obesity, insulin resistance and type 2 diabetes, and cardiovascular disease (thought to be associated with abnormal (usually elevated) amounts of unhealthy lipids such as triglycerides and cholesterol) are a major drain on health systems. Early intervention has the potential to substantially improve health and reduce health expenditure. However, such intervention should ideally be inexpensive and low risk to apply to a large subset of the population. Modulation of the lipid dysregulation by statins represents a proven and attractive option for early intervention; and is arguably one of the most significant developments in terms of health outcomes in the past century. However, statins have only reduced negative cardiovascular outcomes by ~30%, which leaves the majority of the disease burden uncontrolled. Further to this, the dramatic increase in obesity and diabetes (themselves risk factors for cardiovascular disease) has offset much of the risk reduction provided by statins and so new prevention/treatment measures are required.

Lipids are among the least studied molecules of the metabolome. Plasmanyl- and plasmenyl-phospholipids are a unique class of ether phospholipids that are major components of cell membranes. They are characterised by an ether or vinyl-ether linked alkyl chain in the sn1 position and an acyl linked fatty acid in the sn2 position. The structure below shows a plasmenyl-phospholipid, with a vinyl-ether linked 16 carbon alkyl chain in the sn1 position and an acyl linked 18:2 fatty acid in the sn2 position.

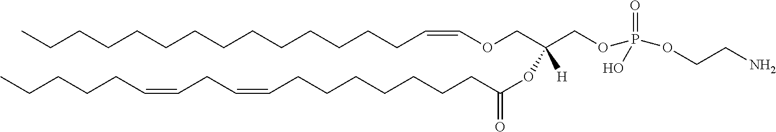 [SMILES: CCCCC/C=C\C/C=C\CCCCCCCC(=O)O[C@H](CO/C=C\CCCCCCCCCCCCCC)COP(=O)(O)OCCN]

### Structure of a Phosphatidylethanolamine Plasmalogen (PE(P-16:0/18:2)

Their biophysical role in cell membranes has been studied while knowledge concerning their biological roles is an important area of new research.

Plasmalogens are primarily present as alkenylphosphatidylcholine (PC(P)) and alkenylphosphatidylethanolamine (PE(P)) species. They are characterised by a cis vinyl ether bond linking an alkyl chain to the sn1 position of the glycerol backbone. They also have an acyl linked fatty acid in the sn2 position. Plasmalogens are often esterified with polyunsaturated fatty acids such as arachidonic acid (20:4) and the omega-3 fatty acid docosahexaenoic acid (22:6, a major constituent of fish oil), whereas the vinyl ether linked residue is usually saturated (i.e. no double bonds present in the chain other than the vinyl ether group) or monounsaturated (i.e. one double bond present in the chain in addition to the vinyl ether group).

Plasmalogen biosynthesis is a complex process involving multiple enzymes within the peroxisome and endoplasmic reticulum. The rate-limiting step in this pathway is the formation of the long chain fatty alcohol by fatty acyl-CoA reductase 1 and 2 (Far-1/2). It is possible to bypass the rate-limiting step in plasmalogen synthesis through the oral administration of naturally occurring alkylglycerols or derivatives thereof (e.g. 1-O-alkylglycerol or 1-O-alkyl-2,3-diacylglycerol). These can be incorporated directly into the phospholipid pathway, and so bypass the peroxisome. This leads to an increase in circulating and tissue plasmalogens.

Although alkylglycerols are present in our diet, the levels in typical diets are insufficient to significantly boost our plasmalogen levels. Shark liver oil is rich in alkylglycerols and is used as a dietary supplement to reduce inflammation and improve immune function. However, the composition of the alkyl chains in shark liver oil is substantially different to the composition of alkenyl chains in human plasmalogens (Paul et al. J Lipid Res. 2021; 62:100092).

Alkylglycerols can also be synthesised, providing a future avenue for an environmentally sustainable source of these compounds (Magnusson et al. Tetrahedron. 2011; 67:1821-36; Shi et al. Green Chemistry. 2010; 12(12)).

A complex process controls the plasmalogen composition with respect to the ratio of different alkenyl chains present at the sn1 position and different acyl chains present at the sn2 position of the plasmalogen. The process depends on the availability of fatty acids for conversion into fatty alcohols in the early steps of the ether linked alkyl chain and the availability of fatty acids for direct incorporation and remodelling of the sn2 acyl linkage. While the sn2 acyl linkage can be remodelled by the action of phospholipases and acyltransferases within the cells, the sn1 alkenyl linkage has been found to be fixed. Thus, dietary supplementation with precursor molecules such as alkylglycerols (1-O-alkylglycerol or 1-O-alkyl-2,3-diacylglycerol) require appropriate formulations to be effective in modulating the level of plasmalogens while maintaining the tissue specific composition of the alkenyl chains at the sn1 position.

Plasmalogen modulation has previously been targeted to whole body modulation, and so the supplement has typically been formulated to match the composition of plasmalogens in circulation. In human plasma the ratio of three major vinyl ether chains in PE(P) species 16:0, 18:0 and 18:1 is 34:41:25. However, other specific target ratios of 1-O vinyl ether moieties for plasmalogen modulation may be effective for improving other health outcomes or in facilitating the prevention, treatment or risk reduction of particular disorders, diseases, and/or conditions.

There is determined herein a need for alternative ether lipid supplementation programmes that can specifically target other tissues and systems.

Ferroptosis is a type of cell death caused by iron-dependent lipid peroxidation, which disrupts cellular membranes and produces oxidised phospholipids (Jiang, X., Stockwell, B. R. & Conrad, M. “Ferroptosis: mechanisms, biology and role in disease,” Nature Reviews Molecular Cell Biology 22, 266-282 (2021)). The exact mechanism of ferroptosis and its relationship to certain types of cancer is still unclear, although the morphological, genetic, and chemical characteristics of ferroptotic cells are very distinguishable from other causes of cell death such as apoptosis and necroptosis (Chen, Z., Wang, W., Abdul Razak, S. R. et al., “Ferroptosis as a potential target for cancer therapy,” Cell Death Dis 14, 460 (2023)).

There are numerous surveillance methods to protect the cells from unwanted ferroptosis, including glutathione peroxidase (GPX-4), which reduces ferroptosis-derived lipid peroxidases into alcohols (Jiang, X., Stockwell, B. R. & Conrad, M. “Ferroptosis: mechanisms, biology and role in disease,” Nature Reviews Molecular Cell Biology 22, 266-282 (2021)). Other enzymes that attenuate ferroptosis include ferroptosis suppressor protein 1, GTP cyclohydrase-1 and dihydroorotate dehydrogenase (Kist, M and Vucic, D. “Cell death pathways: intricate connections and disease implications,” The EMBO Journal 40, e106700 (2021)). Certain diseases such as cancer, inflammatory diseases, bacterial infections, pulmonary fibrosis, hepatitis, inflammatory bowel disease, neurodegenerative diseases, kidney injury, ischemia-reperfusion injury, skeletal muscle injury, Alzheimer's disease, and frailty in the elderly may be treated by blocking ferroptosis (see, e.g., Zhang, C. Liu, X., Jin, S., Chen, Y., Guo, R. “Ferroptosis in cancer therapy: a novel approach to reversing drug resistance,” Molecular Cancer 21, (2022); Kist, M and Vucic, D. “Cell death pathways: intricate connections and disease implications,” The EMBO Journal 40, e106700 (2021)); Singh, G., Kesharwani, P., Singh, G. K., Kumar, S., Putta, A., Modi, G., “Ferroptosis and its modulators: A raising target for cancer and Alzheimer's disease,” Bioorg. Med. Chem., 98, p. 117564 (2024); and Chavoshinezhad, S., Beirami, E., Izadphanah, E., Feligioni, M., Hassanzadeh, K., “Molecular mechanism and potential therapeutic targets of necroptosis and ferroptosis in Alzheimer's disease” Biomedicine & Pharmacotherapy 168, p. 115656 (2023).

### SUMMARY

The present inventors have undertaken extensive research into the composition of plasmalogens in various tissues and from this research have identified immune cells as a target for plasmalogen modulation. Controlled modulation of the plasmalogen composition in immune cells can provide health benefits, such as prevention, treatment and/or risk reduction of associated diseases, disorders, and/or conditions thereof. A controlled plasmalogen modulation of immune cells can also be provided using a supplement formulated to maintain an effective composition of immune cell plasmalogen sn1 alkenyl chains to achieve beneficial biological function. Further advantages can be provided using unique compositions of alkyl and alkenyl sn1 ether chains for modulating for all immune cell types. Particular compositions can also be formulated to target specific immune cell types. An understanding of plasmalogen composition in immune cells has therefore enabled development of formulations of plasmalogens and/or alkylglycerols as dietary supplements to provide for a coordinated increase in the plasmalogen level in immune cells, or for specific immune cell types, while generally maintaining the composition of the alkenyl chains at the sn1 position.

In one embodiment, there is provided a composition for increasing in vivo ether lipids in immune cells comprising an ether lipid molecule of Compound 1:

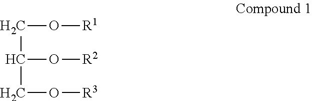

wherein
R¹ is an alkyl or alkenyl group;
R² is hydrogen or

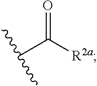

R³ is hydrogen,

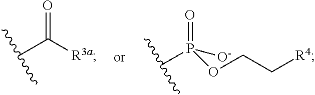

R^{2a} and R^{3a} are each an alkyl or alkenyl group; and
R⁴ is —N(Me)₃⁺ or —NH₃⁺.

In some embodiments, there is provided a method of increasing in vivo ether lipids in immune cells comprising administering to a subject an effective amount of a composition according to any aspects, embodiments, or examples thereof as described herein.

In some embodiments, there is provided a method of treating or preventing a disease, disorder, or condition, associated with the immune system in a subject comprising administering to a subject an effective amount of a composition according to any aspects, embodiments, or examples thereof as described herein.

In some embodiments, there is provided a method of improving immune health or an immune response in a subject comprising administering to a subject an effective amount of a composition according to any aspects, embodiments, or examples thereof as described herein. In some embodiments, there is provided a method of improving response to vaccination in a subject comprising administering to a subject an effective amount of a composition according to any aspects, embodiments, or examples thereof as described herein.

In some embodiments, there is provided a method of treating or preventing an adverse response to a viral infection (e.g. COVID 19) in a subject comprising administering to a subject an effective amount of a composition according to any aspects, embodiments, or examples thereof as described herein.

In some embodiments, there is provided a method of treating or preventing a disease, disorder, or condition, associated with ferroptosis of immune cells in a subject comprising administering to a subject an effective amount of a composition according to any aspects, embodiments, or examples thereof as described herein.

In some embodiments, there is provided a use of a composition according to any aspects, embodiments, or examples thereof as described herein, for the manufacture of a medicament for improving immune health or an immune response in a subject in need thereof, or for treating or preventing an adverse response to a vaccination and/or viral infection in a subject.

In some embodiments, there is provided a use of a composition according to any aspects, embodiments, or examples thereof as described herein, for the manufacture of a medicament for treating or preventing a disease, disorder, or condition, associated with ferroptosis of immune cells in a subject.

It will be appreciated that other aspects, embodiments, or examples, of the compositions and/or methods may be provided according to any aspects, embodiments, or examples thereof as described below and herein.

### BRIEF DESCRIPTION OF THE FIGURES

The accompanying drawings, which are incorporated into and form a part of the specification, illustrate several embodiments of the present disclosure and, together with the description, serve to explain the principles of the disclosure. Unless specified otherwise, data are presented with box plots where the centre line within each box denotes the median value, boxes extend from the 25^{th} to the 75^{th} percentile of each cell type's distribution of values, the whiskers represent the minimum and maximum values, and the circles beyond these whiskers represent outliers.

FIG. 1 shows levels of total alkenylphosphatidylethanolamine (PE(P)) in human immune cell subtypes. Immune cell subsets were isolated from n=9-14 individual donors for each cell type and lipidomic analysis was performed. Phosphatidylethanolamine plasmalogens (PE(P)) were expressed relative to the total lipidome.

FIG. 2 shows levels of total alkenylphosphatidylethanolamine with 16:0 alkenyl chain (PE(P-16:0/xx)) in different human immune cell subtypes. Immune cell subsets were isolated from n=9-14 individual donors for each cell type and lipidomic analysis was performed. Phosphatidylethanolamine plasmalogens with 16:0 alkenyl chain (PE(P-16:0/xx)) were expressed relative to the total lipidome.

FIG. 3 shows levels of total alkenylphosphatidylethanolamine with 18:0 alkenyl chain (PE(P-18:0/xx)) in different human immune cell subtypes. Immune cell subsets were isolated from n=9-14 individual donors for each cell type and lipidomic analysis was performed. Phosphatidylethanolamine plasmalogens with 18:0 alkenyl chain (PE(P-18:0/xx)) were expressed relative to the total lipidome.

FIG. 4 shows levels of total alkenylphosphatidylethanolamines with 18:1 alkenyl chain (PE(P-18:1/xx)) in different human immune cell subtypes. Immune cell subsets were isolated from n=9-14 individual donors for each cell type and lipidomic analysis was performed. Phosphatidylethanolamine plasmalogens with 18:1 alkenyl chain (PE(P-18:1/xx)) were expressed relative to the total lipidome.

FIG. 5 shows relative proportion of alkenylphosphatidylethanolamines with 16:0 alkenyl chain (PE(P-16:0/xx)) in human immune cell subtypes. Immune cell subsets were isolated from n=9-14 individual donors for each cell type and lipidomic analysis was performed. The proportion of phosphatidylethanolamine plasmalogens with 16:0 alkenyl chain (PE(P-16:0/xx)) was expressed relative to the total level of PE(P-16:0/xx), PE(P-18:0/xx) and PE(P-18:1/xx).

FIG. 6 shows relative proportion of alkenylphosphatidylethanolamines with 18:0 alkenyl chain (PE(P-18:0/xx)) in human immune cell subtypes. Immune cell subsets were isolated from n=9-14 individual donors for each cell type and lipidomic analysis was performed. The proportion of phosphatidylethanolamine plasmalogens with 18:0 alkenyl chain (PE(P-18:0/xx)) was expressed relative to the total level of PE(P-16:0/xx), PE(P-18:0/xx) and PE(P-18:1/xx).

FIG. 7 shows relative proportion of alkenylphosphatidylethanolamine with 18:1 alkenyl chain (PE(P-18:1/xx)) in different human immune cell subtypes. Immune cell subsets were isolated from n=9-14 individual donors for each cell type and lipidomic analysis was performed. The proportion of phosphatidylethanolamine plasmalogens with 18:1 alkenyl chain (PE(P-18:1/xx)) was expressed relative to the total level of PE(P-16:0/xx), PE(P-18:0/xx) and PE(P-18:1/xx).

FIG. 8 shows levels of total alkenylphosphatidylethanolamine (PE(P)) in mouse immune cell subtypes. Immune cell subsets were isolated from n=8-10 individual mice for each cell type and lipidomic analysis was performed. Phosphatidylethanolamine plasmalogens (PE(P)) were expressed relative to the total lipidome.

FIG. 9 shows levels of total alkenylphosphatidylethanolamines with 16:0 alkenyl chain (PE(P-16:0/xx)) in mouse immune cell subtypes. Immune cell subsets were isolated from n=8-10 individual mice for each cell type and lipidomic analysis was performed. Phosphatidylethanolamine plasmalogens with 16:0 alkenyl chain (PE(P-16:0/xx)) were expressed relative to the total lipidome.

FIG. 10 shows levels of total alkenylphosphatidylethanolamines with 18:0 alkenyl chain (PE(P-18:0/xx)) in mouse immune cell subtypes. Immune cell subsets were isolated from n=8-10 individual mice for each cell type and lipidomic analysis was performed. Phosphatidylethanolamine plasmalogens with 18:0 alkenyl chain (PE(P-18:0/xx)) were expressed relative to the total lipidome.

FIG. 11 shows levels of total alkenylphosphatidylethanolamines with 18:1 alkenyl chain (PE(P-18:1/xx)) in mouse immune cell subtypes. Immune cell subsets were isolated from n=8-10 individual mice for each cell type and lipidomic analysis was performed. Phosphatidylethanolamine plasmalogens with 18:1 alkenyl chain (PE(P-18:1/xx)) were expressed relative to the total lipidome.

FIG. 12 shows relative proportion of alkenylphosphatidylethanolamines with 16:0 alkenyl chain (PE(P-16:0/xx)) in mouse immune cell subtypes. Immune cell subsets were isolated from n=8-10 individual mice for each cell type and lipidomic analysis was performed. The proportion of phosphatidylethanolamine plasmalogens with 16:0 alkenyl chain (PE(P-16:0/xx)) was expressed relative to the total level of PE(P-16:0/xx), PE(P-18:0/xx) and PE(P-18:1/xx).

FIG. 13 shows relative proportion of alkenylphosphatidylethanolamines with 18:0 alkenyl chain (PE(P-18:0/xx)) in mouse immune cell subtypes. Immune cell subsets were isolated from n=8-10 individual donors for each cell type and lipidomic analysis was performed. The proportion of phosphatidylethanolamine plasmalogens with 18:0 alkenyl chain (PE(P-18:0/xx)) was expressed relative to the total level of PE(P-16:0/xx), PE(P-18:0/xx) and PE(P-18:1/xx).

FIG. 14 shows relative proportion of alkenylphosphatidylethanolamines with 18:1 alkenyl chain (PE(P-18:1/xx)) in mouse immune cell subtypes. Immune cell subsets were isolated from n=8-10 individual donors for each cell type and lipidomic analysis was performed. The proportion of phosphatidylethanolamine plasmalogens with 18:1 alkenyl chain (PE(P-18:1/xx)) was expressed relative to the total level of PE(P-16:0/xx), PE(P-18:0/xx) and PE(P-18:1/xx).

FIG. 15 shows effect of shark liver oil supplementation on the alkenylphosphatidylethanolamine (PE(P)) level of circulatory white cells in humans. Ten male participants were randomised into placebo or shark liver oil treatment arms and received 4 g shark liver oil per day or placebo (methylcellulose) for 3 weeks followed by a 3-week washout phase and were then crossed over to 3 weeks of the alternate placebo/shark liver oil treatment. Lipidomic analysis was performed on the circulatory white cells. Box plots represent the levels of phosphatidylethanolamine plasmalogen (PE(P)) (relative to total phosphatidylcholine) before and after placebo/shark liver oil treatment. The nominal significance of the treatment effect was determined using Repeated Measures ANOVA; *** indicates p<0.001.

FIG. 16 shows effect of shark liver oil supplementation on the levels alkenylphosphatidylethanolamines with different alkenyl chains of circulatory white cells in humans. Ten male participants were randomised into placebo or shark liver oil treatment arms and received 4 g shark liver oil per day or placebo (methylcellulose) for 3 weeks followed by a 3-week washout phase and were then crossed over to 3 weeks of the alternate placebo/shark liver oil treatment. Lipidomic analysis was performed on the circulatory white cells. Box plots represent the levels of phosphatidylethanolamine plasmalogen (PE(P)) with different alkenyl chains (relative to total phosphatidylcholine) before and after placebo/shark liver oil treatment. The nominal significance of the treatment effect was determined using Repeated Measures ANOVA; *** indicates p<0.001.

FIG. 17 shows effect of shark liver oil supplementation on the alkenyl chain composition of ethanolamine plasmalogens of circulatory white cells in humans. Ten male participants were randomised into placebo or shark liver oil treatment arms and received 4 g shark liver oil per day or placebo (methylcellulose) for 3 weeks followed by a 3-week washout phase and were then crossed over to 3 weeks of the alternate placebo/shark liver oil treatment. Lipidomic analysis was performed on the circulatory white cells. Box plots represent the relative proportions of phosphatidylethanolamine plasmalogen (PE(P)) with different alkenyl chains before and after placebo/shark liver oil treatment.

FIG. 18 shows effect of alkylglycerol mix supplementation on the alkenylphosphatidyletahnolamine (PE(P)) level of circulatory immune cells in mice. C57BL/6NJ mice were provided with ad libitum access to either a standard chow diet supplemented with or without an alkylglycerol mix (0.75% of diet w/w) for 4 weeks (n=8-9 individual mice for each diet groups). After 4 weeks, lipidomic analysis was performed on their circulatory immune cells. Phosphatidylethanolamine plasmalogens (PE(P)) were expressed relative to the total phosphatidylcholine. The mean difference between the groups were evaluated by student t-test; ** indicates p<0.01.

FIG. 19 shows effect of alkylglycerol mix supplementation on the levels of ethanolamine plasmalogens with different alkenyl chains of circulatory immune cells in mice. C57BL/6NJ mice were provided with ad libitum access to either a standard chow diet supplemented with or without an alkylglycerol mix (0.75% of diet w/w) for 4 weeks (n=8-9 individual mice for each diet groups). After 4 weeks, lipidomic analysis was performed on their circulatory immune cells. The levels of phosphatidylethanolamine plasmalogens (PE(P)) with different alkenyl chains were expressed relative to the total phosphatidylcholine. The mean difference between the groups were evaluated by student t-test; *** indicates p<0.001.

FIG. 20 shows effect of alkylglycerol mix supplementation on the alkenyl chain composition ethanolamine plasmalogens of circulatory immune cells in mice. C57BL/6NJ mice were provided with ad libitum access to either a standard chow diet supplemented with or without an alkylglycerol mix (0.75% of diet w/w) for 4 weeks (n=8-9 individual mice for each diet groups). After 4 weeks, lipidomic analysis was performed on their circulatory immune cells. The relative proportions of phosphatidylethanolamine plasmalogens (PE(P)) with different alkenyl chains were expressed as a percentage of the total level of PE(P-16:0/xx), PE(P-18:0/xx) and PE(P-18:1/xx).

FIG. 21 shows effect of alkylglycerol mix (AKG) supplementation on the (A) viability and (B) lipid peroxidation in mouse T cells. C57BL/6N male mice were fed with a chow diet with or without 0.75% alkylglycerol mix for 4 weeks and then their T cells were isolated and subsequently treated with different concentrations of ML210 (0-10 μM) for 24 hours. Following this, the proportion of viable T cells (A) and the level of total lipid peroxides in T cells (B) were measured. Data are presented as mean±standard error of mean (n=5-6/group). Data was analysed using a one-way ANOVA with Tukey's HSD test. *p<0.05 and ***p<0.001.

FIG. 22 shows effect of alkylglycerol mix (AKG) supplementation on the (A) toll-like receptor 4 (TLR4) dimerization and (B) tumour necrosis factor alpha (TNFα) level. Bone marrow derived macrophages (BMDMs) were isolated from male C57BL/6N mice and treated with alkylglycerol mix for 48 hours and then with lipopolysaccharide for 15 min. Following this, the proportion of TLR4 dimers in cells (A) and the level of TNFα in the culture media (B) were measured. Data are presented as mean±standard deviation (n=3/group). The mean difference between the groups were evaluated by student t-test.

FIG. 23 shows experimental plan to examine the effect of alkylglycerol supplementation in NP-OVA protein immunisation. WT: wild-type; T-KO: Cd4-CreGPx4^{flox/flox}, NP-OVA: −4-Hydroxy-3-nitrophenylacetyl hapten conjugated to ovalbumin; T_{FH}: follicular helper T cells; GPx4: glutathione peroxidase (Gpx4); ICOS: inducible T-cell costimulatory; IL-21: interleukin-21; ROS: reactive oxygen species; B_{GC}: germinal center B cells.

FIG. 24 shows experimental plan to examine the effect of alkyl glycerol supplementation against viral infection. WT: wild-type; T-KO: Cd4-CreGPx4^{flox/flox}

FIG. 25 shows experimental plan to examine the effect of alkylglycerol supplementation in vaccine mediated protection against viral infection. WT: wild-type; T-KO: Cd4-CreGPx4^{flox/flox-}

### DETAILED DESCRIPTION

The present disclosure describes the following various non-limiting embodiments, which relate to substantial research undertaken into identifying and developing ether lipid compositions for providing a coordinated increase in ether lipid levels in vivo in immune cells of a subject.

### Terms

Unless defined otherwise, all technical and scientific terms used herein have the same meaning as commonly understood by those of ordinary skill in the art to which the disclosure belongs. As used herein the singular forms “a”, “an” and “the” include plural aspects unless the context clearly dictates otherwise. Thus, for example, reference to “a lipid species” includes a single lipid species, as well as two or more lipid species, reference to “the disclosure” includes single and multiple aspects of the disclosure and so forth.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. As used herein, the singular form “a”, “an” and “the” include singular and plural references unless the context indicates otherwise.

The term “and/or”, e.g., “X and/or Y” shall be understood to mean either “X and Y” or “X or Y” and shall be taken to provide explicit support for both meanings or for either meaning. As used herein, the term “about”, unless stated to the contrary, refers to +/−10%, or +/−5%, of the designated value. In other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise stated. No language in the specification should be construed as indicating any non-claimed element as essential to the practice of the disclosure.

The naming convention for lipids used here follows the guidelines established by the Lipid Maps Consortium and the shorthand notation of Liebisch et al. (Liebisch et al., Fahy et. al. (2009), Fahy et al. (2005)]. Phospholipids typically contain two fatty acid chains and in the absence of detailed characterisation are expressed as the sum composition of carbon atoms and double bonds (i.e. PC (38:6)). However, where an acyl chain composition has been determined the naming convention indicates this (i.e. PC (38:6) is changed to PC (16:0_22:6)). This is also extended into other lipid classes or subclasses. Species separated chromatographically but incompletely characterised were labelled with an (a) or (b), for example PC(P-17:0/20:4) (a) and (b) where (a) and (b) represent the elution order.

The present disclosure refers to lipid molecules using the numbering system X:Y. The number X represents the number of carbon atoms present in the chain.

In the context of alkylglycerols, alkyacylglycerols or alkyldiacylglycerols, the number Y represents the number of double bonds present in the chain. For example, an alkylglycerol numbered as 16:0 contains a hydrocarbon group having a 16 carbon chain with no double bonds. As a further example, an alkylglycerol numbered as 18:1 contains a hydrocarbon group having an 18 carbon chain with 1 double bond.

In the context of plasmalogens/plasmenyl phospholipids, the number Y in the first listed alkenyl chain (i.e. PE(P-X:Y/X:Y) represents the number of double bonds present in the alkenyl chain in addition to the vinyl ether group. For example, a plasmalogen numbered as PE(P-16:0/20:4) the 16:0 alkenyl group contains a hydrocarbon group having a 16 carbon chain with no double bonds other than the vinyl ether group (i.e. there is a double bond between the first 2 carbons and the remaining 14 carbons are saturated). As another example, a plasmalogen numbered as PE(P-18:1/20:4) the 18:1 alkenyl group contains a hydrocarbon group having an 18 carbon chain with 1 double bond in addition to the vinyl ether group (i.e. there is a double bond between the first 2 carbon atoms, and there is one other double bond between 2 carbons out of the remaining 16 carbons).

Where ether lipids contain one or more double bonds, the double bonds may be located at various positions in the hydrocarbon chains. For example, an alkylglycerol numbered as 18:1 may contain a mixture of species, e.g. with cis-n7 and cis-n9 double bonds. As another example, a plasmalogen (e.g PE(P)) numbered as 18:1 may contain a mixture of species, e.g. with cis-n7 and cis-n9 double bonds.

As used herein, the term “plasmanyl” shall be understood to refer to phospholipids having an ether bond in the sn-1 position to an alkyl group.

As used herein, the term “plasmenyl” shall be understood to refer to phospholipids having an ether bond in the sn-1 position to an alkenyl group. The plasmenyl phospholipids are referred to as “plasmalogens”.

A plasmalogen having a “16:0” alkenyl group is typically a molecule having an ether bond in the sn-1 position to an 16 carbon chain which contains a double bond between carbons 1 and 2 (i.e. typically a cis-vinyl ether group), and no other double bonds in the chain.

A plasmalogen having an “18:0” alkenyl group is typically a molecule having an ether bond in the sn-1 position to an 18 carbon chain which contains a double bond between carbons 1 and 2 (i.e. typically a cis-vinyl ether group), and no other double bonds in the chain.

A plasmalogen having an “18:1” alkenyl group is typically a molecule having an ether bond in the sn-1 position to an 18 carbon chain which contains a double bond between carbons 1 and 2 (i.e. typically a cis-vinyl ether group), and having one additional double bond, typically between carbons 7 and 8 (e.g. n7), between carbons 9 and 10 (e.g. n9), or between carbons 11 and 12 (e.g. n11), and typically a cis-double bond.

A plasmalogen having an “18:2” alkenyl group is typically a molecule having an ether bond in the sn-1 position to an 18 carbon chain which contains a double bond between carbons 1 and 2 (i.e. typically a cis-vinyl ether group), and having two additional double bonds, typically between carbons 9 and 10, and between carbons 11 and 12, and typically cis-double bonds.

A plasmalogen having an “18:2” acyl alkenyl group is typically a molecule having an ester bond in the sn-2 position to an 18 carbon chain which has two double bonds, typically between carbons 9 and 10, and between carbons 11 and 12, and typically cis-double bonds.

A plasmalogen having a “20:4” acyl alkenyl group is typically a molecule having a ester bond in the sn-2 position to a 20 carbon chain which has four double bonds, typically between carbons 5 and 6, carbons 8 and 9, carbons 11 and 12, and carbons 14 and 15, and typically cis-double bonds.

As used herein, “acyl” refers to a group having a straight, branched, or cyclic configuration or a combination thereof, attached to the parent structure through a carbonyl functionality. Such groups may be saturated or unsaturated, aliphatic or aromatic, and carbocyclic or heterocyclic. Examples of a C₁-C₂₄acyl-group include acetyl, benzoyl-, nicotinoyl-, propionyl-, isobutyryl-, oxalyl-, and the like. Lower-acyl refers to acyl groups containing one to four carbons. An acyl group can be unsubstituted or substituted, for example with one or more groups selected from halogen, —OH, —NH₂, —CN, —OC₁₋₄alkyl and —CO₂H. Additional examples or generally applicable substituents are illustrated by the specific compounds described herein.

The term “aliphatic” as used herein, includes saturated, unsaturated, straight chain (i.e., unbranched), or branched, aliphatic hydrocarbons, which are optionally substituted with one or more functional groups. In some embodiments, the aliphatic may contain one or more functional groups such as double bond, triple bond, or a combination thereof. As will be appreciated by one of ordinary skill in the art, “aliphatic” is intended herein to include, but is not limited to, alkyl, alkenyl, alkynyl, or acyl moieties. Thus, as used herein, the term “alkyl” includes straight and branched saturated groups. An analogous convention applies to other generic terms such as “alkenyl”, “alkynyl”, “acyl” and the like. Furthermore, as used herein, the terms “alkyl”, “alkenyl”, “alkynyl”, “acyl” and the like encompass both substituted and unsubstituted groups.

As used herein, “alkenyl” refers to a straight or branched chain hydrocarbon containing, for example, from 2 to 30 carbons and containing at least one carbon-carbon double bond. In some embodiments, the alkenyl group contains 10 to 25, 14 to 22, or 16 to 20 carbon atoms. In some embodiments, the alkenyl group contains 15, 16, 17, 18, 19 or 20 carbon atoms. Representative examples of “alkenyl” include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, 3-undecenyl, 4-dodecenyl, 4-tridecenyl, 9-tetradecenyl, 8-pentadecenyl, 5-hexadecenyl, 8-heptadecenyl, 9-octadecenyl, 9-nonadecenyl and the like. Additional examples or generally applicable substituents are illustrated by the specific compounds described herein.

As used herein, “alkyl” refers to a straight or branched chain hydrocarbon containing, for example, from 1 to 30 carbon atoms. In some embodiments, the alkyl group contains 10 to 25, 14 to 22, or 16 to 20 carbon atoms. In some embodiments, the alkyl group contains 15, 16, 17, 18, 19 or 20 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, noctyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl and the like. Additional examples or generally applicable substituents are illustrated by the specific compounds described herein.

As used herein, “acyl alkenyl” refers to a straight or branched chain hydrocarbon containing, for example, from 2 to 30 carbons and containing at least one carbon-carbon double bond, which is covalently bonded to an acyl group. The use of nomenclature 22:6 or 18:2 and the like in the context of an acyl alkenyl group refers to an acyl alkenyl group having 22 carbons or 18 carbons respectively, and having 6 or 2 double bonds respectively. An example of an acyl alkenyl group is:

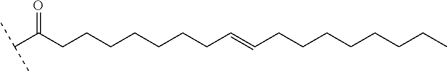

Acyl alkenyl groups may be present in species such as alkylacylglycerols or alkyldiacylglycerols (as an acyl group), or as an acyl group in plasmanyl- or plasmenyl-phospholipids. Typically, when present in those species, there is no double bond between the carbons which are α- and β- to the acyl group.

As used herein, “acyl alkyl” refers to a straight or branched chain hydrocarbon containing, for example, from 1 to 30 carbons, which is covalently bonded to an acyl group. The use of nomenclature 22:0 or 18:0 and the like in the context of an acyl alkyl group refers to an acyl alkyl group having 22 carbons or 18 carbons respectively. An example of an acyl alkyl group is:

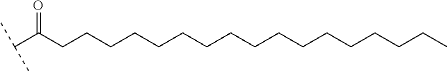

It will also be recognized that the compounds described herein may possess asymmetric centres and are therefore capable of existing in more than one stereoisomeric form. The disclosure thus also relates to compounds in substantially pure isomeric form at one or more asymmetric centres e.g., greater than 90% ee, such as 95% or 97% ee or greater than 99% ee, as well as mixtures, including racemic mixtures, thereof. Such isomers may be naturally occurring or may be prepared by asymmetric synthesis, for example using chiral intermediates, or by chiral resolution.

The terms “substituted” and “optionally substituted” in reference to alkyl groups, alkenyl groups, or acyl groups refers to the optional substitution of these groups by an additional moiety. In each case, the substituent or additional moiety may be independently selected from hydrogen, C₁₋₄alkyl, and halogen (e.g., Cl, F, Br or I). In certain embodiments, the group is not substituted, i.e., it is unsubstituted.

The present disclosure relates to derivatives of glycerol. Whilst glycerol is achiral, derivatives are typically chiral. Typically the glycerol utilised will have a stereochemical configuration corresponding to that found in nature. In some embodiments, the glycerol derivatives utilised have the following stereochemical configuration:

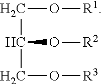

As referred to herein, the term “alkylglycerol” means a compound of Compound 1 in which the R¹ group is a hydrocarbon chain, the R² and R³ groups are each hydrogen. Although the term “alkyl” glycerol is used, it will be understood by those of skill in the art that the term encompasses species with hydrocarbon groups at the R¹ position which include unsaturation in the hydrocarbon chain. However, an alkylglycerol does not contain a double bond between carbons 1 and 2 of the hydrocarbon chain, e.g. proximal to the ether linkage.

An alkylglycerol having a “16:0” group is typically a molecule having an ether bond in the sn-1 position to a 16 carbon saturated hydrocarbon chain, and no double bonds in the chain.

An alkylglycerol having an “18:0” group is typically a molecule having an ether bond in the sn-1 position to an 18 carbon saturated hydrocarbon chain, and no double bonds in the chain.

An alkylglycerol having an “18:1” group is typically a molecule having an ether bond in the sn-1 position to an 18 carbon hydrocarbon chain, which contains one double bond, typically between carbons 9 and 10 and typically a cis-double bond.

As referred to herein, the term “alkylacylglycerol” means a compound of Compound 1 in which the R¹ group is a hydrocarbon chain, one of the R² and R³ groups is hydrogen, and the other of the R² and R³ groups is an acyl group, either an acyl alkyl group or an acyl alkenyl group. Although the term “alkyl” acylglycerol is used, it will be understood by those of skill in the art that the term encompasses species with hydrocarbon groups at the R¹ position which include unsaturation in the hydrocarbon chain. However, an alkylacylglycerol does not contain a double bond between carbons 1 and 2 of the R¹ hydrocarbon chain, e.g. proximal to the ether linkage.

As referred to herein, the term “alkyldiacylglycerol” means a compound of Compound 1 in which the R¹ group is a hydrocarbon chain, and the R² and R³ groups are acyl groups, either acyl alkyl or acyl alkenyl. Although the term “alkyl” diacylglycerol is used, it will be understood by those of skill in the art that the term encompasses species with hydrocarbon groups at the R¹ position which include unsaturation in the hydrocarbon chain. However, an alkyldiacylglycerol does not contain a double bond between carbons 1 and 2 of the R¹ hydrocarbon chain, e.g. proximal to the ether linkage.

The term “extracted” with reference to a particular composition or substance refers to a composition or substance extracted from a natural source, including organisms and parts thereof. For example, lipids or oils extracted from a natural source, refer to lipids or oil that have been separated from other cellular materials, such as the natural source in which the lipid or oil was synthesized. Extracted lipids or oils are obtained through a wide variety of methods, the simplest of which involves physical means alone. For example, mechanical crushing using various press configurations (e.g. screw, expeller, piston, bead beaters, etc.) can separate lipids or oils from cellular materials. Alternately, lipid or oil extraction can occur via treatment with various organic solvents (e.g., hexane), via enzymatic extraction, via osmotic shock, via ultrasonic extraction, via supercritical fluid extraction (e.g., CO₂ extraction), via saponification and via combinations of these methods.

The term “BMI” refers to body mass index, and is calculated by dividing the weight of an individual in kg by their height in metres squared.

Reference to “two or more”, incudes 2, 3, 4, 5, 6, 7, 8, 9 or 10 or more ether lipids.

The following is a list of certain abbreviations used in the disclosure:

| Abbreviation | Description |
|---|---|
| ARA | Arachidonic acid |
| ARDS | Acute respiratory distress syndrome |
| B-cells | B lymphocyte |
| B_{GC} | Germinal center B cells |
| BMI | Body mass index |
| DHA | Docosahexaenoic acid |
| FACS | Fluorescence-activated cell sorting |
| FBS | Foetal bovine serum |
| Gpx4 | Glutathione peroxidase |
| HPLC | High performance liquid chromatography |
| ICOS | Inducible T-cell costimulatory |
| IL-21 | Interleukin-21 |
| ISTD | Internal standard |
| LD50 | Lethal dose (50%) |
| NK cells | Natural killer cells |
| PBMC cells | Peripheral blood mononuclear cells |
| PBS | Phosphate buffered saline |
| PCA | Principal component analysis |
| RBCs | Red blood cells |
| ROS | Reactive oxygen species |
| SCO | Single cell oil |
| T-cells | T lymphocyte |
| T-KO | Cd4-CreGPx4^{flox/flox} or T cell specific GPx4 |
| | knockout mice |
| TNFα | Tumor necrosis factor alpha |
| UHT | Ultra-high temperature processed |
| WT | Wild-type |

### Ether Lipid Compositions

The present inventors have found that ether lipids such as plasmanyl-phospholipids and plasmenyl-phospholipids (e.g. plasmalogens) have in vivo profiles in immune cells that are associated with a healthy state. A coordinated increase in the amounts of ether lipids present in immune cells can provide health benefits, particularly where in vivo profiles (e.g. ratios of key ether lipids) are at least generally maintained. It has also been found that the in vivo ether lipid profile in immune cells can be affected by the administration to subjects of compositions containing ether lipids.

In some embodiments, the immune cells are selected from B-cells, T-cells, NK cells, monocytes, and/or eosiniophila neutrophil. For example, the immune cells may be selected from one or more of Naive B, Memory B, CD4 T Naïve, CD4 T Central Memory, CD4 T Effector Memory, CD8 T Naïve, CD8 T Central Memory, CD8 T Effector Memory, CD56 Dim NK, CD56 Bright NK, Classical Monocyte, Intermediate Monocyte, Non-classical Monocyte, Basophil, Eosinophil, and/or Neutrophil. In one example, the immune cells are T-cells, such as selected from one or more of CD4 T Naïve, CD4 T Central Memory, CD4 T Effector Memory, CD8 T Naïve, CD8 T Central Memory, and/or CD8 T Effector Memory. It has been found that all immune cell types have a common unique ether lipid profile, although the ether lipid profile can vary between each of the immune cell types. The compositions can therefore be targeted more generally to all immune cell types, or more specifically tuned to a subset or individual cell type, for a coordinated increase in key plasmalogens for a specific immune cell type. The compositions can therefore be provided for increasing in vivo ether lipids in immune cells at coordinated ratios associated with a healthy (i.e. non-disease state) of the immune cells.

In some embodiments, the increase of ether lipids in the immune cells (in %) may be at least about 5, 10, 15, 20, 25, 30, 35, 40, 45, 50, 60, 70, 80, 90, 100, 110, 120, 130, 140, 150, 160, 170, 180, 190, or 200. In some embodiments, the increase of ether lipids in the immune cells may be in a range provided by any two of the previous % amounts. It will be appreciated that this increase can be relative to the levels in immune cells prior to administration of the compositions. In some embodiments, the increase in total plasmalogen content in the immune cells is at least about (in mole % of total lipids) 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10. In some embodiments, the increase in total plasmalogen content in the immune cells (in mole % of total lipids) may be in a range provided by any two of the previous % amounts.

The ether lipids increased in the immune cells can be selected from plasmanyl- and/or plasmenyl-phospholipids, for example plasmalogens. The ether lipids increased in the immune cells can be selected from plasmalogens having phosphatidylcholine and/or phosphatidylethanolamine groups. In some embodiments, the ether lipids increased in the immune cells comprise at least two ether lipids of Compound 1 wherein R¹ is selected from C₁₆alkyl, C₁₆alkenyl, C₁₈alkyl, and C₁₈alkenyl. In some embodiments, the ether lipids increased in the immune cells comprise at least two ether lipids of Compound 1 wherein R¹ is selected from C₁₆alkenyl and C₁₈alkenyl, for example plasmalogens having an sn1 group of 16:0, 18:0, and/or 18:1.

Examples of plasmanyl-phospholipids, plasmenyl-phospholipids, and related lipidic species, and their abbreviations, are set out in the table below:

| Description | Abbreviation |
|---|---|
| Alkenylphosphatidylcholine | PC(P) |
| Alkenylphosphatidylethanolamine | PE(P) |
| Alkyl phosphatidylcholine | PC(O) |
| Alkyl phosphatidylethanolamine | PE(O) |
| Lysoalkylphosphatidylcholine | LPC(O) |
| Alkylglycerol | AG |
| Diacylglycerol | DG |
| Triacylglycerol | TG |
| Alkyl-diacylglycerol | TG(O) |
| Alkyl-acylglycerol | DG(O) |
| Fatty acids | FA |

The inventors have identified that healthy subjects have a plasmalogen profile in which certain alkyl or alkenyl ether groups are present in immune cells. For example, a high proportion of ether lipids (i.e. plasmanyl- and/or plasmenyl-phospholipids) having 18:1 alkenyl ether groups, 18:0 alkyl ether groups and 16:0 alkyl ether groups were found in immune cells from a group of healthy subjects. In particular, a high proportion of plasmalogens having 18:1 alkenyl ether groups, 18:0 alkyl ether groups and 16:0 alkyl ether groups were found in immune cells from the group of healthy subjects.

In some embodiments, the compositions comprise an ether lipid molecule of Compound 1:

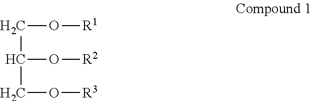

wherein
R¹ is an alkyl or alkenyl group;
R² is hydrogen or

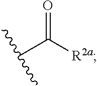

R³ is hydrogen,

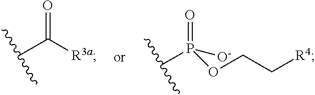

R^{2a} and R^{3a} are each an alkyl or alkenyl group; and
R⁴ is —N(Me)₃⁺ or —NH₃⁺.

In some embodiments, the compositions comprise a mixture of two or more ether lipid molecules of Compound 1 for increasing in vivo ether lipids in immune cells. In some embodiments, the compositions comprise a mixture of three or more ether lipid molecules of Compound 1 for increasing in vivo ether lipids in immune cells. In some embodiments, the compositions can be provided as oral dietary supplements. The ether lipid molecules in the compositions may be provided as plasmanyl- and/or plasmenyl-phospholipids, for example as plasmalogens. The ether lipid molecules in the compositions may be provided as plasmalogen precursors, for example alkyl and/or alkenyl glycerols. It will be appreciated that Compound 1 as described herein can cover plasmalogens (i.e. where R¹ is an alkenyl group providing a saturated or unsaturated vinyl ether, R² is a saturated or unsaturated acyl group, and R³ is a phosphoryl group) and/or plasmalogen precursors, for example alkylglycerols. It will be appreciated that the compositions can be formulated to include mixtures or blends of plasmalogens and/or plasmalogen precursors, such as alkylglycerols.

Ether lipids of Compound 1 can include alkyl glycerols, alkenyl glycerols, alkyl acyl glycerols, alkenyl acyl glycerols, alkyl diacyl glycerols, alkenyl diacyl glycerols, and ether phospholipids such as plasmanyl-phospholipids and plasmenyl-phospholipids. In some embodiments, the ether lipids of Compound 1 are selected from the group consisting of alkyl glycerols, alkenyl glycerols, alkyl acyl glycerols, alkenyl acyl glycerols, alkyl diacyl glycerols and, alkenyl diacyl glycerols (i.e. in which case R² is hydrogen or

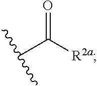

and R³ is hydrogen or

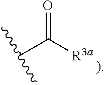

In some embodiments, the ether lipids of Compound 1 are alkyl glycerols (i.e. in which case R² and R³ are hydrogen). It will be appreciated that alkylglycerols are lipids with a glycerol backbone, to which fatty acid or fatty acid derivatives are coupled by means of an ether bond instead of the ester bond that characterizes most mono-, di- and tri-glycerols and related phospholipids (see, e.g., U.S. Pat. No. 6,121,245, which is incorporated herein by reference in its entirety).

In some embodiments, R¹ is selected from a C₁₈alkyl, C₁₆alkenyl, C₁₈alkyl, and C₁₈alkenyl. In some embodiments, R¹ is selected from a C₁₆alkenyl and C₁₈alkenyl. In some embodiments, the R¹ alkenyl group is a saturated or unsaturated vinyl group, such as present in plasmalogens. It will be appreciated that an unsaturated vinyl group will have one or more additional double bonds in the carbon chain in addition to the “vinyl” double bond group. In some embodiments, the composition comprises ether lipids wherein R² and R³ is hydrogen (e.g. alkylglycerols).

In some embodiments, the composition comprises ether lipids in which R² is hydrogen and R³ is

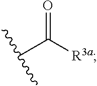

and R^{3a} is selected from the group consisting of a saturated alkyl hydrocarbon, a monounsaturated alkenyl hydrocarbon and a polyunsaturated alkenyl hydrocarbon (e.g. alkyl acyl glycerol).

In some embodiments, the composition comprises ether lipids in which R³ is hydrogen and R² is

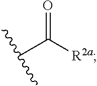

and R^{2a} is selected from the group consisting of a saturated alkyl hydrocarbon, a monounsaturated alkenyl hydrocarbon and a polyunsaturated alkenyl hydrocarbon (e.g. alkyl acyl glycerol).

In some embodiments, the composition comprises ether lipids in which R² is:

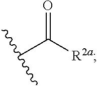

### R³ is

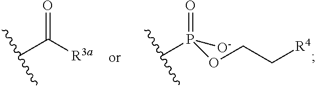

wherein R^{2a} is selected from the group consisting of a saturated alkyl hydrocarbon, a monounsaturated alkenyl hydrocarbon and a polyunsaturated alkenyl hydrocarbon; R^{3a} is selected from the group consisting of a saturated alkyl hydrocarbon, a monounsaturated alkenyl hydrocarbon and a polyunsaturated alkenyl hydrocarbon; and R⁴ is —N(Me)₃⁺ or —NH₃⁺ (e.g. alkyl diacyl glycerol, PC or PE plasmanyl- or plasmenyl-phospholipid).

In embodiments where an alkylglycerol is administered, or where an alkylacyl glycerol is administered, or where an alkyldiacylglycerol is administered, references to an alkyl or alkenyl R¹ group having the numbering X:Y means that the group has X carbons, and has Y double bonds.

In embodiments where a plasmalogen is administered, references to an alkyl or alkenyl R¹ group having the numbering X:Y means that the group has X carbons, and has Y double bonds in addition to the vinyl ether.

In some embodiments, the composition comprises an ether lipid molecule of Compound 1 wherein R¹ is selected from the group consisting of C₁₆alkyl, C₁₆alkenyl, C₁₈alkyl, and C₁₈alkenyl. In some embodiments, the composition comprises an ether lipid molecule of Compound 1 wherein R¹ is selected from the group consisting of C₁₆alkyl, C₁₈alkyl, and C₁₈alkenyl. In some embodiments, the composition comprises an ether lipid molecule of Compound 1 wherein R¹ is selected from 16:0 alkyl, 18:0 alkyl, and 18:1 alkenyl.

In some embodiments, the composition comprises at least two ether lipid molecules of Compound 1 wherein R¹ is selected from the group consisting of C₁₆alkyl, C₁₆alkenyl, C₁₈alkyl, and C₁₈alkenyl. In some embodiments, the composition comprises at least two ether lipid molecules of Compound 1 wherein R¹ is selected from the group consisting of C₁₆alkyl, C₁₈alkyl, and C₁₈alkenyl. In some embodiments, the composition comprises at least two ether lipid molecules of Compound 1 wherein R¹ is selected from 16:0 alkyl, 18:0 alkyl, and 18:1 alkenyl.

In some embodiments, the composition comprises an ether lipid molecule of Compound 1 wherein R¹ is selected from C₁₆alkyl or C₁₆alkenyl. In some embodiments, the composition comprises an ether lipid molecule of Compound 1 wherein R¹ is 16:0 alkyl. In some embodiments, the ratio of the C16 alkyl ether lipid molecules to all other ether lipid molecules in the composition can be greater than about 1:20, 1:15, 1:10, 1:5, 1:2. 1:1, 2:1, 3:2, 4:1, 5:1, 6:1, 7:1, 8:1, 9:1, or 10:1. The ratio of the C₁₆alkyl ether lipid molecules to all other ether lipid molecules in the composition can be provided in a range provided by any two of these previous amounts, for example in a range of about 1:20 to 10:1, 1:10, 9:1, or 1:1 to 8:1. In some embodiments, a molar percent of C₁₆alkyl ether lipid molecules based on total lipid content in the compositions can be provided in an amount of at least about 35, 40, 45, 50, 55, 60, 65, 70, 75, or 80. In some embodiments, a molar percent of C₁₆alkyl ether lipid molecules (e.g. 16:0) based on total lipid content in the compositions can be provided in the range two of the previous amounts, for example from about 35% to 80%, 40% to 70%, or 45% to 65%.

In some embodiments, the composition comprises at least one ether lipid molecule of Compound 1 wherein R¹ is selected from C₁₆alkyl or C₁₆alkenyl, and at least one ether lipid molecule of Compound 1 wherein R¹ is selected from C₁₈alkyl or C₁₈alkenyl. In some embodiments, the composition comprises an ether lipid molecule of Compound 1 wherein R¹ is 16:0 alkyl and an ether lipid molecule of Compound 1 wherein R¹ is 18:0 alkyl. In some embodiments, the ratio of the C₁₆alkyl ether lipid molecules to the C18 alkyl ether lipid molecules can be greater than about 0.8:1, 0.85:1, 0.9:1, 0.95:1, 1:1, 1.25:1, 1.5:1, 1.75:1, 2:1, 2.25:1, 2.5:1, 2.75:1, 3:1, 3.25:1, 3.5:1, 3.75:1, or 4:1. The ratio of the C₁₆alkyl ether lipid molecules to the C18 alkyl ether lipid molecules can be provided in a range provided by any two of these previous amounts, for example in a range of about 0.85:1 to 3:1, 1:1 to 2.5:1, 1.5:1 to 2:1, or 1.75:1 to 2:1. In some embodiments, a molar percent of C₁₆alkyl ether lipid molecules based on total lipid content in the compositions can be provided in an amount of at least about 35, 40, 45, 50, 55, 60, 65, 70, 75, or 80. In some embodiments, a molar percent of C₁₆alkyl ether lipid molecules (e.g. 16:0) based on total lipid content in the compositions can be provided in the range two of the previous amounts, for example from about 35% to 80%, 40% to 70%, or 45% to 65%. In some embodiments, a molar percent of C₁₈ alkyl ether lipid molecules (e.g. 18:0) based on total lipid content in the compositions can be provided in an amount of at least about 15, 20, 25, 30, 35, 40, 45, 50, 55, or 60, 65, or 70. In some embodiments, a molar percent of 18:0 alkyl ether lipid molecules based on total lipid content in the compositions can be provided in the range provided by any two of the previous values, for example in a range from about 15% to 60%, 20% to 50%, or 25% to 45%.

In some embodiments, the composition comprises an ether lipid molecule of Compound 1 wherein R¹ is 16:0 alkyl and an ether lipid molecule of Compound 1 wherein R¹ is 18:1 alkenyl. In some embodiments, the ratio of the 16:0 alkyl ether lipid molecules to the 18:1 alkenyl ether lipid molecules can be greater than about 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1; 9.5:1, or 10:1. The ratio of the 16:0 alkyl ether lipid molecule to the 18:1 alkenyl ether lipid molecule can be provided in a range provided by any two of these previous amounts, for example in a range of about 2:1 to 10:1, 2:8, 3:1 to 7:1, 4:1 to 7:1, or 4:1 to 6:1. In some embodiments, a molar percent of 16:0 alkyl ether lipid molecules based on total lipid content in the compositions can be provided in an amount of at least about 35, 40, 45, 50, 55, 60, 65, 70, 75, or 80. In some embodiments, a molar percent of 16:0 alkyl ether lipid molecules based on total lipid content in the compositions can be provided in the range two of the previous amounts, for example from about 35% to 80%, 40% to 70%, or 45% to 65%. In some embodiments, a molar percent of 18:1 alkyl ether lipid molecules based on total lipid content in the compositions can be provided in an amount of less than about 30, 25, 20, 15, 10, 5, or 1. In some embodiments, a molar percent of 18:1 alkenyl ether lipid molecules based on total lipid content in the compositions can be in the range provided by any two of the previous amounts, for example in a range from about 1% to 20%, 2% to 18%, or 5% to 15%.

In some embodiments, the composition comprises an ether lipid molecule of Compound 1 wherein R¹ is 18:0 alkyl and an ether lipid molecule of Compound 1 wherein R¹ is 18:1 alkenyl. The ratio of the 18:0 alkyl ether lipid molecule to the 18:1 alkenyl ether lipid molecule can be provided in a ratio of at least about 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1; 9.5:1, or 10:1. The ratio of the 18:0 alkyl ether lipid molecule to the 18:1 alkenyl ether lipid molecule can be in a range provided by any two of the previous amounts, for example in a range of about 1.5:1 to 10:1, 2:1 to 7:1, or 3:1 to 6:1. In some embodiments, a molar percent of 18:0 alkyl ether lipid molecules based on total lipid content in the compositions can be provided in an amount of at least about 15, 20, 25, 30, 35, 40, 45, 50, 55, or 60, 65, or 70. In some embodiments, a molar percent of 18:0 alkyl ether lipid molecules based on total lipid content in the compositions can be provided in the range provided by any two of the previous values, for example in a range from about 15% to 60%, 20% to 50%, or 25% to 45%. In some embodiments, a molar percent of 18:1 alkyl ether lipid molecules based on total lipid content in the compositions can be provided in an amount of less than about 30, 25, 20, 15, 10, 5, or 1. In some embodiments, a molar percent of 18:1 alkenyl ether lipid molecules based on total lipid content in the compositions can be in the range provided by any two of the previous amounts, for example in a range from about 1% to 20%, 2% to 18%, or 5% to 15%.

In some embodiments, the composition comprises an ether lipid molecule of Compound 1 wherein R¹ is 16:0 alkyl, an ether lipid molecule of Compound 1 wherein R¹ is 18:0 alkyl, and an ether lipid molecule of Compound 1 wherein R¹ is 18:1 alkenyl. In some embodiments, a molar percent of 16:0 alkyl ether lipid molecules based on total lipid content in the compositions can be provided in an amount of at least about 35, 40, 45, 50, 55, 60, 65, 70, 75, or 80. In some embodiments, a molar percent of 16:0 alkyl ether lipid molecules based on total lipid content in the compositions can be provided in the range two of the previous amounts, for example from about 35% to 80%, 40% to 70%, or 45% to 65 In some embodiments, a molar percent of 18:0 alkyl ether lipid molecules based on total lipid content in the compositions can be provided in an amount of at least about 15, 20, 25, 30, 35, 40, 45, 50, 55, or 60, 65, or 70. In some embodiments, a molar percent of 18:0 alkyl ether lipid molecules based on total lipid content in the compositions can be provided in the range provided by any two of the previous values, for example in a range from about 15% to 60%, 20% to 50%, or 25% to 45%. In some embodiments, a molar percent of 18:1 alkyl ether lipid molecules based on total lipid content in the compositions can be provided in an amount of less than about 30, 25, 20, 15, 10, 5, or 1. In some embodiments, a molar percent of 18:1 alkenyl ether lipid molecules based on total lipid content in the compositions can be in the range provided by any two of the previous amounts, for example in a range from about 1% to 20%, 2% to 18%, or 5% to 15%. In some embodiments, the composition has a molar percent of 16:0 alkyl ether lipid molecules in the range of from 35% to 80%, 40% to 70%, or 45% 65%, a molar percent of 18:0 alkyl ether lipid molecules in the range of from 15% to 60%, 20% to 50%, or 25% to 45%, and a molar percent of 18:1 alkenyl ether lipid molecules in the range of from about 1% to 20%, 2% to 18%, or 5% to 15%. It will be appreciated that any of the above ratios and/or molar percentage amounts may also be applicable.

In some embodiments, the ether lipids having a C₁₆alkyl R¹ group, C₁₆alkenyl R¹ group, C₁₈ alkyl R¹ group, and/or C18 alkenyl R¹ group, if present, together comprise (in % of total ether lipids in composition) at least about 10, 20, 30, 40, 50, 60, 70, 80, 90, 95, or 98, or 99. In In some embodiments, the ether lipids having an 18:1 alkenyl R¹ group, ether lipids having an 18:0 alkyl R¹ group, and/or ether lipids having a 16:0 alkyl R¹ group, if present, together comprise (in % of total ether lipids in composition) at least about 10, 20, 30, 40, 50, 60, 70, 80, 90, 95, or 98, or 99.

In some embodiments, the ether lipids having a C₁₆alkyl R¹ group, C₁₆alkenyl R¹ group, C₁₈ alkyl R¹ group, and/or C₁₈ alkenyl R¹ group, if present, together or individually comprise (in % of total lipids in composition) at least about 5, 10, 20, 30, 40, 50, 60, 70, 80, 90, 95, or 98, or 99. In some embodiments, the ether lipids having an 18:1 alkenyl R¹ group, ether lipids having an 18:0 alkyl R¹ group, and/or ether lipids having a 16:0 alkyl R¹ group, if present, together or individually comprise (in % of total lipids in composition) at least about 5, 10, 20, 30, 40, 50, 60, 70, 80, 90, 95, or 98, or 99.

The compositions according to any aspects, embodiments, or examples thereof, as described herein may be provided in a product in an amount (weight %) of at least about 0.01, 0.1, 1, 5, 10, 15, 20, 25, 30, 35, 40, 45, 50, 55, 60, 65, 70, 75, or 80. The compositions according to any aspects, embodiments, or examples thereof, as described herein may be provided in a product in an amount (weight %) of less than about 90, 85, 80, 75, 70, 65, 60, 55, 50, 45, 40, 35, 30, 25, 20, 15, 10, 5, or 1. The compositions according to any aspects, embodiments, or examples thereof, as described herein may be provided in a product in range amount (weight %) provided by any two of the previous minimum and/or maximum amounts, for example between about 0.01 and 70, 1 and about 60, or between about 5% and about 50%.

The composition may be provided in the form of a product, which may be a dietary supplement, capsule, syrup, liquid, food or beverage. In some embodiments, the product may comprise or consist of the compositions according to any aspects, embodiments, or examples thereof, as described herein. In some embodiments the products or compositions may further comprise optional additives and/or excipients.

In some embodiments, the composition can be prepared by mixing a plurality of ether lipids in ratios and/or levels corresponding with ratios and/or levels associated with a non-disease state in vivo immune cell. In some embodiments, the total plasmalogen content in the immune cells of a subject prior to treatment is less than about 10, 9, 8, 7, 6, 5, 4, 3, or 2 molar % of total lipids.

In some embodiments, the ether lipids mixed for administration are alkylglycerols. In some embodiments, the alkylglycerols have a structure of Compound 1. In some embodiments, the alkylglycerols have a structure of Compound 1A.

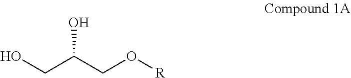

In some embodiments R refers to a C₁-C₃₀ alkyl, or alkenyl chain. In some embodiments, R refers to a C₁₆alkyl chain. In some embodiments, R refers to a C₁₈alkyl chain. In some embodiments, R refers to a C₁₈alkenyl chain.

In embodiments where an alkylglycerol is administered, or where an alkylacyl glycerol is administered, or where an alkyldiacylglycerol is administered, references to an alkyl or alkenyl R group having the numbering X:Y means that the group has X carbons, and has Y double bonds.

In embodiments where a plasmalogen is administered, references to an alkyl or alkenyl R group having the numbering X:Y means that the group has X carbons, and has Y double bonds in addition to the vinyl ether.

in some embodiments, the composition comprises an ether lipid molecule of Compound 1A wherein R is selected from the group consisting of C₁₆alkyl, C₁₆alkenyl, C₁₈alkyl, and C₁₈alkenyl. In some embodiments, the composition comprises an ether lipid molecule of Compound 1A wherein R is selected from the group consisting of C₁₆alkyl, C₁₈alkyl, and C₁₈alkenyl. In some embodiments, the composition comprises an ether lipid molecule of Compound 1A wherein R is selected from 16:0 alkyl, 18:0 alkyl, and 18:1 alkenyl.

In some embodiments, the composition comprises at least two ether lipid molecules of Compound 1A wherein R is selected from the group consisting of C₁₆alkyl, C₁₆alkenyl, C₁₈alkyl, and C₁₈alkenyl. In some embodiments, the composition comprises at least two ether lipid molecules of Compound 1A wherein R is selected from the group consisting of C₁₆alkyl, C₁₈alkyl, and C₁₈alkenyl. In some embodiments, the composition comprises at least two ether lipid molecules of Compound 1A wherein R is selected from 16:0 alkyl, 18:0 alkyl, and 18:1 alkenyl.

In some embodiments, the ether lipids comprising chimyl alcohol, batyl alcohol, and/or selachyl alcohol if present, together or individually comprise (in % of total lipids in composition) at least about 5, 10, 20, 30, 40, 50, 60, 70, 80, 90, 95, or 98, or 99.

In some embodiments, the composition is mixture of two or more ether lipid molecules of Compound 1A. In some embodiments, the mixture comprises two or more ether lipid molecules selected from chimyl alcohol, batyl alcohol and selachyl alcohol. In some embodiments, the mixture of two or more ether lipids comprises chimyl alcohol. In some embodiments, the mixture of two or more ether lipids comprises chimyl alcohol in an amount of 19-55% (either molar percent or weight percent) of the total ether lipids. In some embodiments, the mixture of two or more ether lipids comprises batyl alcohol. In some embodiments, the mixture of two or more ether lipids comprises batyl alcohol in an amount of 5-35% (either molar percent or weight percent) of the total ether lipids. In some embodiments, the mixture of two or more ether lipids comprises selachyl alcohol. In some embodiments, the mixture of two or more ether lipids comprises selachyl alcohol in an amount of 10-76% (either molar percent or weight percent) of the total ether lipids. In some embodiments, the mixture of two or more ether lipids comprises chimyl alcohol in an amount of 19-55% (either molar percent or weight percent) of the total ether lipids, batyl alcohol in an amount of 5-35% (either molar percent or weight percent) of the total ether lipids, and selachyl alcohol in an amount of 10-76% (either molar percent or weight percent) of the total ether lipids.

In some embodiments, the mixture of two or more ether lipid molecules of Compound 1A comprises a mixture of chimyl alcohol and batyl alcohol. In some embodiments, the mixture of two or more ether lipids comprises a mixture of chimyl alcohol in an amount of 19-55% (either molar percent or weight percent) of the total ether lipids and batyl alcohol in an amount of 5-35% (either molar percent or weight percent) of the total ether lipids. In some embodiments, the mixture of two or more ether lipid molecules of Compound 1A comprises a mixture of chimyl alcohol and selachyl alcohol. In some embodiments, the mixture of two or more ether lipids comprises a mixture of chimyl alcohol in an amount of 19-55% (either molar percent or weight percent) of the total ether lipids and selachyl alcohol in an amount of 10-76% (either molar percent or weight percent) of the total ether lipids. In some embodiments, the mixture of two or more ether lipid molecules of Compound 1A comprises a mixture of batyl alcohol and selachyl alcohol. In some embodiments, the mixture of two or more ether lipid molecules of Compound A comprises a mixture of batyl alcohol in an amount of 5-35% (either molar percent or weight percent) of the total ether lipids and selachyl alcohol in an amount of 10-76% (either molar percent or weight percent) of the total ether lipids. In some embodiments, the mixture of two or more ether lipid molecules of Compound A comprises chimyl alcohol in an amount of 19-55% (either molar percent or weight percent) of the total ether lipids, batyl alcohol in an amount of 5-35% (either molar percent or weight percent) of the total ether lipids, and selachyl alcohol in an amount of 10-76% (either molar percent or weight percent) of the total ether lipids.

In some embodiments, the mixture of two or more ether lipid molecules of Compound 1A comprises chimyl alcohol, batyl alcohol and selachyl alcohol. In some embodiments the chimyl alcohol is present in an amount of 19% (either molar percent or weight percent) of the total ether lipids, batyl alcohol is present in an amount of 5% (either molar percent or weight percent) of the total ether lipids, and selachyl alcohol is present in an amount of 76% (either molar percent or weight percent) of the total ether lipids. In some embodiments the chimyl alcohol is present in an amount of 50% (either molar percent or weight percent) of the total ether lipids, batyl alcohol is present in an amount of 30% (either molar percent or weight percent) of the total ether lipids, and selachyl alcohol is present in an amount of 20% (either molar percent or weight percent) of the total ether lipids. In some embodiments the chimyl alcohol is present in an amount of 55% (either molar percent or weight percent) of the total ether lipids, batyl alcohol is present in an amount of 35% (either molar percent or weight percent) of the total ether lipids, and selachyl alcohol is present in an amount of 10% (either molar percent or weight percent) of the total ether lipids.

In some embodiments, the composition comprises an ether lipid molecule of Compound 1A wherein R is selected from C₁₆alkyl or C₁₆alkenyl. In some embodiments, the composition comprises an ether lipid molecule of Compound 1A wherein R is 16:0 alkyl. In some embodiments, the ratio of the C₁₆alkyl ether lipid molecules to all other ether lipid molecules in the composition can be greater than about 1:20, 1:15, 1:10, 1:5, 1:2. 1:1, 2:1, 3:2, 4:1, 5:1, 6:1, 7:1, 8:1, 9:1, or 10:1. The ratio of the C₁₆alkyl ether lipid molecules to all other ether lipid molecules in the composition can be provided in a range provided by any two of these previous amounts, for example in a range of about 1:20 to 10:1, 1:10, 9:1, or 1:1 to 8:1 In some embodiments, a molar percent of C₁₆alkyl ether lipid molecules based on total lipid content in the compositions can be provided in an amount of at least about 35, 40, 45, 50, 55, 60, 65, 70, 75, or 80. In some embodiments, a molar percent of C₁₆alkyl ether lipid molecules (e.g. 16:0) based on total lipid content in the compositions can be provided in the range two of the previous amounts, for example from about 35% to 80%, 40% to 70%, or 45% to 65%.

In some embodiments, the composition comprises at least one ether lipid molecule of Compound 1A wherein R is selected from C₁₆alkyl or C₁₆alkenyl, and at least one ether lipid molecule of Compound 1A wherein R is selected from C₁₈alkyl or C₁₈alkenyl. In some embodiments, the composition comprises an ether lipid molecule of Compound 1A wherein R is 16:0 alkyl and an ether lipid molecule of Compound 1A wherein R is 18:0 alkyl. In some embodiments, the ratio of the C₁₆alkyl ether lipid molecules to the C₁₈alkyl ether lipid molecules can be greater than about 0.8:1, 0.85:1, 0.9:1, 0.95:1, 1:1, 1.25:1, 1.5:1, 1.75:1, 2:1, 2.25:1, 2.5:1, 2.75:1, 3:1, 3.25:1, 3.5:1, 3.75:1, or 4:1. The ratio of the C₁₆alkyl ether lipid molecules to the C₁₈alkyl ether lipid molecules can be provided in a range provided by any two of these previous amounts, for example in a range of about 0.85:1 to 3:1, 1:1 to 2.5:1, 1.5:1 to 2:1, or 1.75:1 to 2:1. In In some embodiments, a molar percent of C₁₆alkyl ether lipid molecules based on total lipid content in the compositions can be provided in an amount of at least about 35, 40, 45, 50, 55, 60, 65, 70, 75, or 80. In some embodiments, a molar percent of C₁₆alkyl ether lipid molecules (e.g. 16:0) based on total lipid content in the compositions can be provided in the range two of the previous amounts, for example from about 35% to 80%, 40% to 70%, or 45% to 65%. In some embodiments, a molar percent of C₁₈alkyl ether lipid molecules (e.g. 18:0) based on total lipid content in the compositions can be provided in an amount of at least about 15, 20, 25, 30, 35, 40, 45, 50, 55, or 60, 65, or 70. In some embodiments, a molar percent of 18:0 alkyl ether lipid molecules based on total lipid content in the compositions can be provided in the range provided by any two of the previous values, for example in a range from about 15% to 60%, 20% to 50%, or 25% to 45%.

In some embodiments, the composition comprises an ether lipid molecule of Compound 1A wherein R is 16:0 alkyl and an ether lipid molecule of Compound 1A wherein R is 18:1 alkenyl. In some embodiments, the ratio of the 16:0 alkyl ether lipid molecules to the 18:1 alkenyl ether lipid molecules can be greater than about 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1; 9.5:1, or 10:1. The ratio of the 16:0 alkyl ether lipid molecule to the 18:1 alkenyl ether lipid molecule can be provided in a range provided by any two of these previous amounts, for example in a range of about 2:1 to 10:1, 2:8, 3:1 to 7:1, 4:1 to 7:1, or 4:1 to 6:1. In some embodiments, a molar percent of 16:0 alkyl ether lipid molecules based on total lipid content in the compositions can be provided in an amount of at least about 35, 40, 45, 50, 55, 60, 65, 70, 75, or 80. In some embodiments, a molar percent of 16:0 alkyl ether lipid molecules based on total lipid content in the compositions can be provided in the range two of the previous amounts, for example from about 35% to 80%, 40% to 70%, or 45% to 65%. In some embodiment, a molar percent of 18:1 alkyl ether lipid molecules based on total lipid content in the compositions can be provided in an amount of less than about 30, 25, 20, 15, 10, 5, or 1. In some embodiments, a molar percent of 18:1 alkenyl ether lipid molecules based on total lipid content in the compositions can be in the range provided by any two of the previous amounts, for example in a range from about 1% to 20%, 2% to 18%, or 5% to 15%.

In some embodiments, the composition comprises an ether lipid molecule of Compound 1A wherein R is 18:0 alkyl and an ether lipid molecule of Compound 1A wherein R is 18:1 alkenyl. The ratio of the 18:0 alkyl ether lipid molecule to the 18:1 alkenyl ether lipid molecule can be provided in a ratio of at least about 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1; 9.5:1, or 10:1. The ratio of the 18:0 alkyl ether lipid molecule to the 18:1 alkenyl ether lipid molecule can be in a range provided by any two of the previous amounts, for example in a range of about 1.5:1 to 10:1, 2:1 to 7:1, or 3:1 to 6:1. In some embodiments, a molar percent of 18:0 alkyl ether lipid molecules based on total lipid content in the compositions can be provided in an amount of at least about 15, 20, 25, 30, 35, 40, 45, 50, 55, or 60, 65, or 70. In some embodiments, a molar percent of 18:0 alkyl ether lipid molecules based on total lipid content in the compositions can be provided in the range provided by any two of the previous values, for example in a range from about 15% to 60%, 20% to 50%, or 25% to 45%. In some embodiments, a molar percent of 18:1 alkyl ether lipid molecules based on total lipid content in the compositions can be provided in an amount of less than about 30, 25, 20, 15, 10, 5, or 1. In some embodiments, a molar percent of 18:1 alkenyl ether lipid molecules based on total lipid content in the compositions can be in the range provided by any two of the previous amounts, for example in a range from about 1% to 20%, 2% to 18%, or 5% to 15%.

In some embodiments, the composition comprises an ether lipid molecule of Compound 1A wherein R is 16:0 alkyl, an ether lipid molecule of Compound 1A wherein R is 18:0 alkyl, and an ether lipid molecule of Compound 1A wherein R is 18:1 alkenyl. In some embodiments, a molar percent of 16:0 alkyl ether lipid molecules based on total lipid content in the compositions can be provided in an amount of at least about 35, 40, 45, 50, 55, 60, 65, 70, 75, or 80. In some embodiments, a molar percent of 16:0 alkyl ether lipid molecules based on total lipid content in the compositions can be provided in the range two of the previous amounts, for example from about 35% to 80%, 40% to 70%, or 45% to 65%. In some embodiments, a molar percent of 18:0 alkyl ether lipid molecules based on total lipid content in the compositions can be provided in an amount of at least about 15, 20, 25, 30, 35, 40, 45, 50, 55, or 60, 65, or 70. In some embodiments, a molar percent of 18:0 alkyl ether lipid molecules based on total lipid content in the compositions can be provided in the range provided by any two of the previous values, for example in a range from about 15% to 60%, 20% to 50%, or 25% to 45 In some embodiments, a molar percent of 18:1 alkyl ether lipid molecules based on total lipid content in the compositions can be provided in an amount of less than about 30, 25, 20, 15, 10, 5, or 1. In some embodiments, a molar percent of 18:1 alkenyl ether lipid molecules based on total lipid content in the compositions can be in the range provided by any two of the previous amounts, for example in a range from about 1% to 20%, 2% to 18%, or 5% to 15%. In some embodiments, the composition has a molar percent of 16:0 alkyl ether lipid molecules in the range of from 35% to 80%, 40% to 70%, or 45% 65%, a molar percent of 18:0 alkyl ether lipid molecules in the range of from 15% to 60%, 20% to 50%, or 25% to 45%, and a molar percent of 18:1 alkenyl ether lipid molecules in the range of from about 1% to 20%, 2% to 18%, or 5% to 15%. It will be appreciated that any of the above ratios and/or molar percentage amounts may also be applicable.

In some embodiments, the ether lipids having a C₁₆alkyl R group, C₁₆alkenyl R group, C₁₈ alkyl R group, and/or C₁₈alkenyl R group, if present, together comprise (in % of total ether lipids in composition) at least about 10, 20, 30, 40, 50, 60, 70, 80, 90, 95, or 98, or 99. In some embodiments, the ether lipids having an 18:1 alkenyl R group, ether lipids having an 18:0 alkyl R group, and/or ether lipids having a 16:0 alkyl R group, if present, together comprise (in % of total ether lipids in composition) at least about 10, 20, 30, 40, 50, 60, 70, 80, 90, 95, or 98, or 99.

In some embodiments, the ether lipids having a C₁₆alkyl R group, C₁₆alkenyl R group, C₁₈alkyl R group, and/or C₁₈alkenyl R group, if present, together or individually comprise (in % of total lipids in composition) at least about 5, 10, 20, 30, 40, 50, 60, 70, 80, 90, 95, or 98, or 99. In some embodiments, the ether lipids having an 18:1 alkenyl R group, ether lipids having an 18:0 alkyl R group, and/or ether lipids having a 16:0 alkyl R group, if present, together or individually comprise (in % of total lipids in composition) at least about 5, 10, 20, 30, 40, 50, 60, 70, 80, 90, 95, or 98, or 99.

The compositions according to any aspects, embodiments, or examples thereof, as described herein may be provided in a product in an amount (weight %) of at least about 0.01, 0.1, 1, 5, 10, 15, 20, 25, 30, 35, 40, 45, 50, 55, 60, 65, 70, 75, or 80. The compositions according to any aspects, embodiments, or examples thereof, as described herein may be provided in a product in an amount (weight %) of less than about 90, 85, 80, 75, 70, 65, 60, 55, 50, 45, 40, 35, 30, 25, 20, 15, 10, 5, or 1. The compositions according to any aspects, embodiments, or examples thereof, as described herein may be provided in a product in range amount (weight %) provided by any two of the previous minimum and/or maximum amounts, for example between about 0.01 and 70, 1 and about 60, or between about 5% and about 50%.

The composition may be provided in the form of a product, which may be a dietary supplement, capsule, syrup, liquid, food or beverage. In some embodiments, the product may comprise or consist of the compositions according to any aspects, embodiments, or examples thereof, as described herein. The products or compositions may further comprise optional additives and/or excipients.

In some embodiments, the composition can be prepared by mixing a plurality of ether lipids in ratios and/or levels corresponding with ratios and/or levels associated with a non-disease state in vivo immune cell.

In some embodiments the ether lipids mixed for administration are of Compound 1A-I, Compound 1A-II, or Compound 1A-III.

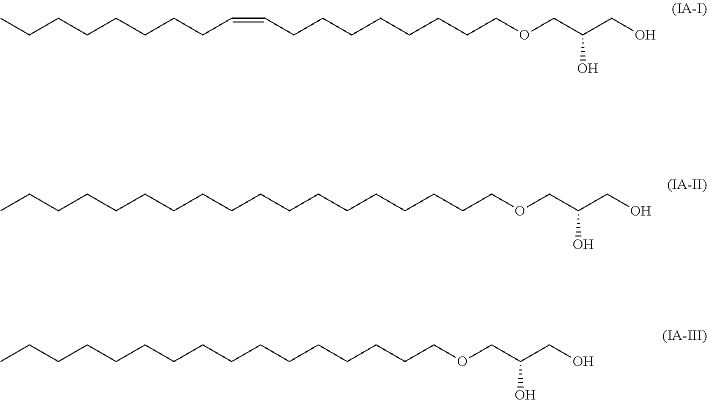 [SMILES: CCCCCCCC/C=C\CCCCCCCCOC[C@@H](O)CO ; CCCCCCCCCCCCCCCCCCOC[C@@H](O)CO ; CCCCCCCCCCCCCCCCOC[C@@H](O)CO]

### Method of Making Compositions

Some aspects of the present disclosure relate to the provision of new compositions containing mixtures of ether lipid molecules of Compound 1. For the avoidance of doubt, the present disclosure relates to new compositions per se, as well as to uses of compositions and methods of using them.

As discussed above, it will be appreciated that the constituents of the formulation may be varied according to the intended purpose of the formulation.

The compositions may be prepared by any suitable means. The composition may for example be prepared by mixing a plurality of ether lipids, in ratios and/or levels associated with a non-disease state immune cells in vivo. The desired amounts of each component of the composition can be combined and blended to provide a uniform mixture.

Ether lipids, and mixtures of ether lipids, may for example be prepared synthetically. Alkyl glycerols (i.e. compounds of Compound 1 wherein R² and R³ are hydrogen), can for example be obtained from commercial sources, and combined to provide a composition having the desired proportions of alkenyl ether and alkyl ether groups. For example, batyl alcohol (an alkyl glycerol having an 18:0 alkyl ether group), also known as C18.0 AKG with a CAS number for the S isomer ((2S)-3-(Octadecyloxy)-1,2-propanediol) of 6129-13-1, is available from Sigma Aldrich. Selachyl alcohol or C18.1 AKG (an alkenyl glycerol having an 18:1 alkenyl ether group, the S isomer of which is also known as (2S)-3-[(9Z)-9-Octadecen-1-yloxy]-1,2-propanediol, CAS 6898-45-9) is available from Alfa Chemistry. C16.0 AKG, chimyl alcohol (CAS 506-03-6) can be sourced from companies such as Cayman Chemical (Item No. 25723) and Avanti Polar Lipids (via Sigma) catalog number 999971P. Bulk quantities of these alkyl glycerols can be found as well for example at EOS Med Chem, BOC Sciences, and Dycn Chem. Alkylglycerols (such as batyl alcohol, chimyl alcohol and selachyl alcohol) may be prepared synthetically. Synthesis of these compounds is well known in the art (see, for instance, Takaishi et al., U.S. Pat. No. 4,465,869, UK U.S. Pat. No. 1,029,610, and Magnusson et al., Tetrahedron (2011) 67, or WO2013/071418, which are hereby incorporated by reference herein in their entirety). In addition, mono- and di-esters of alkylglycerols are well-known in the art and their syntheses have been described (see, e.g., Burgos et al. (1987), J. Org. Chem. 52:4973-4977; Hirth et al. (1982) Helv. Chim. Acta 65:1059-1084; and Hirth et al. (1983) Helv. Chim. Acta 66:1210-1240).

Plasmalogens may be prepared synthetically. Synthesis of these compounds is well known in the art (see, for instance, Shin et al. (2003) J Org. Chem., 2003 68(17): 6760-6766; Van den Bossche, et al. (2007) J. Org. Chem. 72 (13): 5005-5007 and Khan et al., International Publication No. WO 2013/071418, which are incorporated herein by reference in their entirety).

Chiral ether lipids may be used in racemic, enantiomerically enriched, or enantiomerically pure forms. For example, some commercially available ether lipids are provided as mixtures of enantiomers. However, ether lipids obtained from natural sources are typically obtained as a single enantiomer. In some embodiments, chiral ether lipids present in the composition are present as a single enantiomer (e.g. the R form, or the S form). In some embodiments, chiral ether lipids present in the composition as a mixture of enantiomers (e.g. in racemic form).

It will be appreciated that, whilst some embodiments of the present disclosure relate to the use of novel compositions, that some known compositions containing a mixture of ether lipids of Compound 1 may also be of use in increasing in vivo ether lipid levels and/or ratios in immune cells. Accordingly, in some embodiments, the present disclosure relates to methods and/or uses utilising existing ether lipid compositions. For example, alkylglycerols may be extracted from a natural source, illustrative examples of which include fish oils such as shark oils, and hematopoietic organs such as bone marrow and spleen. In specific embodiments, alkylglycerols are extracted from fish liver oils, particularly liver oils of elasmobranch fish such as sharks (e.g., Greenland shark, dogfish, ratfish, rabbitfish see, e.g., Hallgren et al., U.S. Pat. No. 4,046,914, which is incorporated by reference herein in its entirety), rays, Seamouse etc. Shark liver oil may be obtained commercially (see, e.g., ALKYROL, Eurohealth, Inc., Parkside, Pa.). Common fatty alcohols found in shark liver oil are chimyl alcohol, batyl alcohol and selachyl alcohol. Non-limiting methods for extracting alkylglycerols are disclosed for example in Hallgren et al. (supra) and Brohult et al., International Publication No. WO 1998/52550, which is incorporated by reference herein in its entirety).

Plasmalogens may be prepared from any suitable source. For example, they may be extracted from a natural source, such as but not limited to microorganisms and animals. Non-limiting examples of plasmalogen-producing microorganisms anaerobic bacteria, suitably from the family Acidaminococcaceae, which are intestinal bacteria. Representative examples of plasmalogen-producing animals include birds, mammals, fishes, shellfishes, and the like. In some embodiments, the mammals are livestock mammals, representative examples of which include cow, pig, horse, sheep, goat, and the like. Suitable plasmalogen-containing mammalian tissues include skin, spinal cord, brain, intestines, heart, genitals, and the like. Examples of birds include chicken, domestic duck, quail, duck, pheasant, ostrich, turkey, and the like. There is no particular limitation to an avian tissue to be used. For example, bird meat (in particular, bird's breast meat), bird skin, internal organs of birds, bird eggs etc., are suitably used. Two or more types of different tissues from one or more species of organisms may be used in combination. Methods for extracting plasmalogens are known in the art, non-limiting examples of which are described in Nishimukai et al. (2003) Lipids 38(12): 1227-1235, Herrmann et al., U.S. Pat. No. 4,613,621 and Mawatari et al., U.S. Publication No. 2013/0172293, which are incorporated herein by reference in their entirety.

In some embodiments, the composition comprises at least one isolated compound. In some embodiments, the at least one isolated compound is present in the composition at a purity of greater than 99%. In some embodiments, the at least one isolated compound is present in the composition at a purity of greater than 99.9%.

In some embodiments the composition comprises a mixture of at least two compounds wherein the % (w/v) of one compound is at least 90%.

### Products

The composition may be an emulsion, suspension, or other mixture, and can be combined with one or more other ingredients to form a product.

The product may be a cream, gel, tablet, liquid, pill, capsule, or extruded product.

The product may be a food, food ingredient, drink ingredient, nutritional composition, cosmetic or cosmetic ingredient.

The food may be animal feed, aquaculture feed.

The product may be a food ingredient for e.g. infant formulae, children formula, adult formula, yoghurts, beverages, elderly supplement, ultra-high temperature processed (UHT) drinks (e.g. milk), soup, dips, pasta products, bread, snacks and other bakery products processed cheese, and/or animal feed (including aquaculture feed).

In some embodiments, the composition is in the form of a composition for addition to a food or beverage. In some embodiments, the composition is in the form of a product, which is a dietary supplement, capsule, liquid, syrup, food or beverage. For example, a subject may take a capsule containing the composition as a health or nutritional composition, e.g. on a daily basis. As a further example, the ether lipids may be incorporated into a health food product such as a nutrition bar.

A further embodiment contemplated by the present disclosure is a formulation, which may be in the form of a food, such as a dietary supplement, that, upon ingestion leads to an increased level of plasmalogens within the blood and tissues of the recipient and thereby ameliorates or overcomes any plasmalogen deficiency that may exist within said individual. Increasing the levels of plasmalogens may lead to improved health outcomes. Such a formulation could also be incorporated into a range of foods to facilitate delivery to the recipient. The intended recipients would be anyone who is deficient in plasmalogens and/or who is at risk of any of a range of metabolic diseases where plasmalogens may play a protective role.

As used herein, the term “dietary supplement” refers to a food product intended to enhance the diet of the subject and thereby improve nutrition. Dietary supplements may include the compositions described herein alone, or alongside other ingredients intended to supplement the diet, such as vitamins and minerals, fibre, herbs and other botanical extracts including flower remedies, homeopathic remedies, amino acids, enzymes and live microbials, probiotics, prebiotics or any combination thereof. Dietary supplements may be formulated in a wide variety of ways including as oils, gummies, drops, capsules, rapid-melt formulations, lozenges, oral sprays, chewing gums, gels, powders, premixed drinks, meal replacement shakes, or bars.

An example of a foodstuff into which a formulation as described herein can be incorporated, is infant formula. Foods in which a formulation can be incorporated include infant formula, follow-on formula, Medical Foods and Foods for Special Medical Purposes.

An example of a foodstuff into which a composition or formulation as described herein can be incorporated is any food stuff formulated for human consumption.

The terms “Medical Foods” or “Foods for Special Medical Purposes” as used herein refer to foodstuffs that are specially formulated and intended for the dietary management of a disease, disorder or condition that has distinctive nutritional needs that cannot be met by normal diet alone. Medical Foods assist patients who are temporarily or permanently unable to achieve an adequate nutritional intake from normal foods or through modification of the normal diet and are either malnourished or at risk of becoming malnourished. Medical Foods are used under medical supervision and may be administered orally or via tube feeds (e.g., nasogastric tubes). These terms as used herein are referred to under Regulation (EU) No 609/2013 and (Food and Drug Authority (FDA)) 21 CFR 101.9(j)(8)(ii). They are thus distinguished from dietary supplements which are generally available for consumption without medical supervision.

The term “infant” as used herein, is taken to mean a person aged 12 months or younger. The term “infant” is taken to additionally mean “pre-term infant”, which is a person aged 12 months or younger, who was born prior to 36 weeks of gestation. The term “toddler” as used herein, is taken to mean a person greater than one year of age up to three years of age. The term “child” or “children” as used herein, refers to a person greater than three years of age, up to 12 years of age. The terms “infant formula” as used herein, unless otherwise specified, refers to liquid, semi-liquid, solid and semi-solid human milk replacements or substitutes that are suitable for consumption by an infant. The synthetic formulas include components that are of semi-purified or purified origin. As used herein, the terms “semi-purified” and “purified” refer to a material that has been prepared by purification of a natural material or by synthesis. The term “infant formula” is not taken to include unmodified human breast milk.

The infant formula may include liquid and powdered dietary supplements, liquid and powdered human milk fortifiers, liquid and powdered preterm infant formulas, liquid and powdered infant formulas, liquid and powdered elemental and semi-elemental formulas, liquid and powdered toddler formulas, and powdered follow-on formulas suitable for use infants and children. Compositions may be in any product form comprising the ingredients described herein, and which is safe and effective for oral administration.

The infant formula may further include ingredients including, protein, fat, carbohydrate, vitamins, minerals, anti-caking agents, emulsifiers. In some embodiments, the formula may contain purified cow's milk whey, soy protein, fully hydrolysed protein sources, partially hydrolysed protein sources, free amino acids, goat milk proteins, human breast milk proteins, and/or casein as a protein source, a blend of vegetable oils as a fat source, lactose as a carbohydrate source, a vitamin-mineral mix, and other ingredients including, but not limited to, any antioxidants suitable for oral administration such as vitamin A, vitamin E, vitamin C, retinal, tocopherol, L-ascorbyl palmitate, riboflavin and carotenoids, including lutein, beta-carotene, zeaxanthin, and lycopene, and combinations thereof. The infant formula may include oils, for example, vegetable oil including, for example, high oleic sunflower oil, coconut oil, canola oil, sunflower oil, algal oil, oil from fungal sources, or fish oil, and combinations thereof. The oil may be extracted from algal, fish or fungal sources. The oil may be a single cell oil (SCO) extracted from microalgae. The extracted oil may contain docosahexaenoic acid (DHA), arachidonic acid (ARA), and/or long-chain polyunsaturated fatty acid (LCPUFA), and combinations thereof. The infant formula may include starches and starch derivatives, such as tapioca starch, maltodextrins, and dextrose. The infant formula may contain milk-based sugars such as lactose. The infant formula may contain plant-based milks and may contain sugars including fructose or fruit sugar, glucose, or sucrose. The infant formula may include milk products and derivatives from milk, including, for example, lactose, milk proteins, galacto-oligosaccharides, whey concentrate, fructo-oligosaccharides, human milk oligosaccharides, further compounds enriched from milk(s) secreted by mammals including, but not limited to human, bovine, etc.

The infant formula may include anti-caking agents such as tricalcium phosphate, potassium chloride, sodium citrate, and potassium citrate, magnesium hydrogen phosphate, and coagulants, such as for example magnesium chloride, choline chloride, L-ascorbic acid, emulsifier iron (II) sulfate, zinc sulfate.

The powders may be reconstituted with water prior to use to a caloric density tailored to the nutritional needs of the ultimate user, although in most instances the powders are reconstituted with water to form compositions comprising at least 19 kcal/fl oz (660 kcal/liter), more typically from about 20 kcal/fl oz (675-680 kcal/liter) to about 25 kcal/fl oz (820 kcal/liter), even more typically from about 20 kcal/fl oz (675-680 kcal/liter) to about 24 kcal/fl oz (800-810 kcal/liter). Generally, the 22-24 kcal/fl oz formulas are more commonly used in preterm or low birth weight infants, and the 20-21 kcal/fl oz (675-680 to 700 kcal/liter) formulas are more often used in term infants. In some embodiments, the reconstituted powder may have a caloric density of from about 50-100 kcal/liter to about 660 kcal/liter, including from about 150 kcal/liter to about 500 kcal/liter. In some specific embodiments, the emulsion may have a caloric density of 25, or 50, or 75, or 100 kcal/liter.

When the nutritional composition is a powdered infant formula, the protein component is present in an amount of from about 5% to about 35%, including from about 8% to about 12%, and including from about 10% to about 12% by weight of the infant formula; the fat component is present in an amount of from about 10% to about 35%, including from about 25% to about 30%, and including from about 26% to about 28% by weight of the infant formula; and the carbohydrate component is present in an amount of from about 30% to about 85%, including from about 45% to about 60%, including from about 50% to about 55% by weight of the infant formula.

The infant formulas contemplated herein may be formulated to include at least one of fat, protein, and carbohydrate, and preferably also contains vitamins, minerals, and at least one compound of Compound 1, Formula (1A), Formula (1A-I), Formula (1A-II), or Formula (1A-III).

There are many disease settings where plasmalogens have been demonstrated to play a protective role. These include, but are not limited to: metabolic disorders (obesity, insulin resistance, type 2 diabetes; nonalcoholic fatty liver disease, nonalcoholic steatohepatitis); immune related diseases (asthma, atopic dermatitis, type 1 diabetes, infection); cardiovascular disease (atherosclerosis, cardiac remodeling, hypertension); neurological diseases (Alzheimer's disease; Parkinson's disease, autism, amyotrophic lateral sclerosis, frontotemporal dementia; multiple sclerosis; schizophrenia); cancer; myalgic encephalomyelitis/chronic fatigue syndrome; Barth syndrome; peroxisomal disorders (Zellweger syndrome spectrum disorders, rhizomelic chondrodysplasia punctata). These have been reviewed in several recent papers, as follows: Tremblay, et al., “Plasmalogens and platelet-activating factor roles in chronic inflammatory diseases,” BioFactors (2022) 1-14; Schooneveldt, et al., “Ether lipids in obesity: from cells to population studies,” Frontiers in Physiology (March 2022) 13:1-11; Boselli, Jr., et al., “Plasmalogen replacement therapy,” Membranes (2021) 11:838; and, S. Paul, G. I. Lancaster, and P. Meikle, “Plasmalogens: a potential therapeutic target for neurodegenerative and cardiometabolic disease,” Progress Lipid Res. (2019) 74:186-195.

In certain embodiments, the above-described formulations include one or more liquid or gel-based carriers, including, but not limited to, those selected from the group consisting of water and physiological salt solutions, urea, alcohols and derivatives thereof (e.g., methanol, ethanol, propanol, butanol), glycols (e.g., ethylene glycol, propylene glycol), and the like; natural or synthetic flavorings and food-quality coloring agents; thickening agents, including, but not limited to, those selected from the group consisting of corn starch, guar gum, xanthan gum, and the like. In certain embodiments, the one or more liquid or gel-based carrier(s) can be added to the formulations in a weight/volume percentage of from about 0.5% to about 95% weight/volume of the formulation. In certain embodiments, the natural or synthetic flavoring(s) can be added to the formulations in a weight/volume percentage of from about 3.0% to about 10.0% weight/volume of the formulation. In certain embodiments, the coloring agent(s) can be added to the formulations in a weight/volume percentage of from about 1.0% to about 10.0% weight/volume of the formulation. In certain embodiments, the thickening agent(s) can be added to the formulations in a weight/volume percentage of about 2% weight/volume of the formulation.

Reference to a “subject” or “individual” or “patient” includes any human (of any age), primate, mammalian, or other species of veterinary or agricultural importance, or test organism known to the skilled person. Reference to a subject or patient can indicate that the subject has been diagnosed with a condition such as metabolic disease, diabetes, obesity and its sequelae. As used herein, the terms “subject” and “patient” refer to humans or animals to be treated by the methods of the present disclosure. In one embodiment, the subject is a human. In one embodiment, the subject is an animal such as a domestic animal, a working animal or a farm animal. Domestic animals include, but are not limited to, rabbits, birds, cats, dogs, fishes, rats, tortoises, reptiles (lizard, snake) and the like. Working animals include, but are not limited to, cattle, yaks, and horses and the like. Farm animals include, but are not limited to sheep, pigs, cows, chickens, goats, geese, ducks, llamas and the like. In some embodiments, it is envisaged that the compositions described herein will be formulated as an animal feed or dietary supplement for animals. In some embodiments, administration may be oral administration. In some embodiments, administration may be via any appropriate route including a liquid form, a capsule, a tablet, or a lozenge.

Reference to “maintain” or “maintenance” in relation to ether lipids relates to compositions which, for a period of time, retain the ether lipid molecule levels or ratios for the defined molecules at ratios associated with a non-disease state and within plus or minus about 2 SD (standard deviations) in a population. Suitable populations are illustrated in Example 1. In one embodiment, the ether lipids are for providing a coordinated increase in plasmanyl- and/or plasmenyl-phospholipid levels.

Reference to “modulate” or “modify” or the like in relation to ether lipid molecules refers to compositions which, for a period of time, change the ether lipid levels for the defined molecules towards ratios associated with a non-disease state and within plus or minus about 2 SD (standard deviations) in a population. Suitable populations are illustrated in Example 1. In one embodiment, the ether lipids are for modifying plasmanyl- and/or plasmenyl-phospholipid levels and/or ratios.

In one embodiment, modifying of one or more lipid species includes administration of a defined mixture of ether lipid molecules to reduce disease risk factors.

“Reference ether lipid molecule or side chain profile” includes a profile of ether lipid molecules established from a control population, such as a non-disease population or a disease population, or from a particular subject including the subject at an earlier time point. The term “non-disease state” as used herein, refers to a state in which the subject is not suffering from a plasmalogen related disease or deficiency requiring treatment.

Reference to “healthy or non-disease levels or ratios of ether lipid molecules” includes particular molar ratios or proportions or % by weight of two or more ether lipid species determined herein to be associated with a population of healthy humans. The formulations of the present disclosure are useful in maintaining and/or modifying in vivo ether lipid levels at levels and/or ratios associated with a natural, non-disease state (that is, the natural state of a healthy human subject), and/or in modifying or modulating in vivo ether lipid levels towards levels and/or ratios which are associated with a natural, non disease state (that is, the natural state of a healthy human subject). An exemplary, but non-limiting example of the term “healthy human subject” is taken to mean a subject or patient who is not suffering from a disease or disorder associated with a plasmalogen deficiency.

### Methods and Administration of Compositions

Compositions comprising ether lipid molecules as described herein can be administered in an effective amount sufficient to provide a coordinated increase in immune cells of a subject.

In one embodiment, there is provided a method is provided for increasing in vivo ether lipids in immune cells comprising administering to a subject an effective amount of a composition according to any aspects, embodiments, or examples thereof as described herein. In some embodiments, there is provided a composition according to any aspects, embodiments, or examples thereof as described herein for use in increasing in vivo ether lipids in immune cells in a subject. In some embodiments, there is provided a use of a composition according to any aspects, embodiments, or examples thereof as described herein, in the manufacture of a medicament for increasing in vivo ether lipids in immune cells in a subject. The in vivo increase in ether lipids in immune cells can be provided at coordinated ratios associated with a non-disease state of the immune cells.

In some embodiments, the immune cells are selected from B-cells, T-cells, NK cells, monocytes, and/or eosiniophila neutrophil. For example, the immune cells may be selected from one or more of Naive B, Memory B, CD4 T Naïve, CD4 T Central Memory, CD4 T Effector Memory, CD8 T Naïve, CD8 T Central Memory, CD8 T Effector Memory, CD56 Dim NK, CD56 Bright NK, Classical Monocyte, Intermediate Monocyte, Non-classical Monocyte, Basophil, Eosinophil, and/or Neutrophil. In one example, the immune cells are T-cells, such as selected from one or more of CD4 T Naïve, CD4 T Central Memory, CD4 T Effector Memory, CD8 T Naïve, CD8 T Central Memory, and/or CD8 T Effector Memory. It has been found that all immune cell types have a common unique ether lipid profile, although the ether lipid profile can vary between each of the immune cell types. The compositions can therefore be targeted more generally to all immune cell types, or more specifically tuned to a subset or individual cell type, for a coordinated increase in key plasmalogens for a specific immune cell type. The compositions can therefore be provided for increasing in vivo ether lipids in immune cells at coordinated ratios associated with a healthy (i.e. non-disease state) of the immune cells.

As described herein, the ether lipids increased in the immune cells can be selected from plasmanyl- and/or plasmenyl-phospholipids, and in particular from plasmalogens. The plasmalogens can have phosphatidylcholine and/or phosphatidylethanolamine groups. In some embodiments, the ether lipids increased in the immune cells comprise at least two ether lipids of Compound 1 wherein R¹ is selected from C₁₆alkyl, C₁₆alkenyl, C₁₈alkyl, and C₁₈alkenyl. In some embodiments, the ether lipids increased in the immune cells comprise at least two ether lipids of Compound 1 wherein R¹ is selected from C₁₆alkenyl and C₁₈alkenyl, for example plasmalogens having an sn1 group of 16:0, 18:0, and/or 18:1.

It will be appreciated that the ether lipids increased in the immune cells can be provided in coordinated ratios corresponding to the ether lipid molecules as for the compositions according to any embodiments or examples thereof as described herein.

In some embodiments, the method can further comprise (i) identifying an individual with a low amount of plasmalogen content in their immune cells. The method can further comprise (ii) administering to the subject the composition according to any aspects, embodiments, or examples thereof as described herein (e.g. plasmalogens and/or plasmalogen precursors). It will be appreciated that the ether lipid compositions can have coordinated ratios of ether lipid molecules that at least generally correspond to the plasmalogen profile in the immune cells to elevate two or more key plasmalogen species (e.g. 16:0, 18:0 and 18:1) in a coordinated manner for increasing the level of plasmalogens in the immune cells.

A subject's immune cells can be measured for a low amount of total plasmalogen content, or low amount of specific key plasmalogen species (e.g. 16:0, 18:0 and 18:1), to determine if the subject is to be administered the compositions. It will be appreciated that standard techniques such as mass spectrometry can be used to determine such lipid amounts in immune cells. In some embodiments, the total plasmalogen content in the immune cells of a subject is less than about (in mole % of total lipids) 10, 9, 8, 7, 6, 5, 4, 3, 2 or 1. In some embodiments, the plasmalogen content of key plasmalogen species (e.g. 16:0, 18:0 and 18:1), individually or collectively, in the immune cells of a subject is less than about (in mole % of total lipids) 10, 9, 8, 7, 6, 5, 4, 3, 2 or 1. For example, the content of 16:0 plasmalogen in the immune cells of a subject may be less than about (in mole % of total lipids) 10, 9, 8, 7, 6, 5, 4, 3, 2 or 1. For example, the content of 18:0 plasmalogen in the immune cells of a subject may be less than about (in mole % of total lipids) 10, 9, 8, 7, 6, 5, 4, 3, 2 or 1.

In some embodiments the administration of the compositions may increase ether lipids in the immune cells (in % relative to amounts prior to administration) by at least about 5, 10, 15, 20, 25, 30, 35, 40, 45, 50, 60, 70, 80, 90, 100, 110, 120, 130, 140, 150, 160, 170, 180, 190, or 200. The increase of ether lipids in the immune cells may be in a range provided by any two of these previous % amounts. In some embodiments, the administration of the compositions may increase total plasmalogen content in the immune cells by at least about (in mole % of total lipids) 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10. In In some embodiments, the plasmalogen content of key plasmalogen species (e.g. 16:0, 18:0 and 18:1), individually or collectively, in the immune cells of a subject is less than about (in mole % of total lipids) 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10. For example, the content of 16:0 plasmalogen in the immune cells of a subject may be less than about (in mole % of total lipids) 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10. For example, the content of 18:0 plasmalogen in the immune cells of a subject may be less than about (in mole % of total lipids) 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, or 10. In some embodiments, the increase in plasmalogen content in the immune cells (in mole % of total lipids) as previously described may be in a range provided by any two of the previous % amounts.

It will be appreciated that the ether lipid profiles in the compositions can be more specifically formulated or tailored to target specific subclasses or types of immune cell for providing a coordinated increase in key plasmalogens for that specific immune cell subclass or type. In some embodiments, the immune cells are selected from T-cells, for example selected from one or more of CD4 T Naïve, CD4 T Central Memory, CD4 T Effector Memory, CD8 T Naïve, CD8 T Central Memory, and/or CD8 T Effector Memory. The compositions can therefore be provided for increasing in vivo ether lipids in T-cells at coordinated ratios associated with a healthy (i.e. non-disease state) of the T-cells. For example, the composition can comprise an ether lipid molecule of Compound 1 wherein R¹ is selected from C₁₆alkyl or C₁₆alkenyl. In some embodiments, the composition comprises an ether lipid molecule of Compound 1 wherein R¹ is 16:0 alkyl. In some embodiments, the ratio of the C₁₆alkyl ether lipid molecules to all other ether lipid molecules in the composition can be greater than about 1:20, 1:15, 1:10, 1:5, 1:2. 1:1, 2:1, 3:2, 4:1, 5:1, 6:1, 7:1, 8:1, 9:1, or 10:1. The ratio of the C₁₆alkyl ether lipid molecules to the C₁₈alkyl ether lipid molecules can be provided in a range provided by any two of these previous amounts, for example in a range of about 1:20 to 10:1, 1:10, 9:1, or 1:1 to 8:1. In some embodiments, a molar percent of C₁₆alkyl ether lipid molecules based on total lipid content in the compositions can be provided in an amount of at least about 35, 40, 45, 50, 55, 60, 65, 70, 75, or 80. In some embodiments, a molar percent of C₁₆alkyl ether lipid molecules (e.g. 16:0) based on total lipid content in the compositions can be provided in the range two of the previous amounts, for example from about 35% to 80%, 40% to 70%, or 45% to 65%. It will be appreciated that other embodiments or examples of the compositions as described herein can also be applicable to targeting T-cells to provide for a coordinated increase in key plasmalogen species (e.g. 16:0, 18:0 and/or 18:1).

In some embodiments, there is provided a method of treating or preventing a disease, disorder, or condition, associated with the immune system in a subject, the method comprising administering an effective amount of a composition according to any aspects, embodiments, or examples thereof as described herein. In some embodiments, there is provided a composition according to any aspects, embodiments, or examples thereof as described herein for use in treating or preventing a disease, disorder, or condition, associated with the immune system in a subject. In some embodiments, there is provided a use of a composition according to any aspects, embodiments, or examples thereof as described herein in the manufacture of a medicament for treating or preventing a disease, disorder, or condition, associated with the immune system in a subject.

In some embodiments, there is provided a method of improving immune health or an immune response in a subject, the method comprising administering an effective amount of a composition according to any aspects, embodiments, or examples thereof as described herein. In some embodiments, there is provided a composition according to any aspects, embodiments, or examples thereof as described herein for use in improving immune health or an immune response in a subject. In some embodiments, there is provided use of a composition according to any aspects, embodiments, or examples thereof as described herein in the manufacture of a medicament for improving immune health or an immune response in a subject.

In some embodiments, the disease, disorder, or condition in a subject may be a cardiovascular disease, Alzheimer's disease, viral infection (e.g. COVID 19 infection), metabolic disease, diabetes (e.g. type 2 diabetes), cardiovascular disease, obesity, fatty liver disease, an inflammatory condition, or dyslipidaemia.

In some embodiments, there is provided a method of improving response to vaccination in a subject, the method comprising administering an effective amount of a composition according to any aspects, embodiments, or examples thereof as described herein. In some embodiments, there is provided a composition according to any aspects, embodiments, or examples thereof as described herein, for use in improving response to vaccination in a subject. In some embodiments, there is provided use of a composition according to any aspects, embodiments, or examples thereof as described herein, in the manufacture of a medicament for improving response to vaccination in a subject.

In some embodiments, there is provided a method of treating or preventing an adverse response to a viral infection (e.g. COVID 19) in a subject, the method comprising administering to the subject an effective amount of a composition according to any aspects, embodiments, or examples thereof as described herein. The method can provide an enhanced immune response in the subject. In some embodiments, there is provided a composition according to any aspects, embodiments, or examples thereof as described herein, for use in treating or preventing an adverse response to a viral infection (e.g. COVID 19), such as by enhancing an immune response in a subject. In some embodiments, there is provided a composition according to any aspects, embodiments, or examples thereof as described herein, in the manufacture of a medicament for treating or preventing an adverse response to a viral infection (e.g. COVID 19), such as by enhancing an immune response in a subject.

In some embodiments, there is provided a method of treating or preventing a disease, disorder, or condition, associated with ferroptosis of immune cells in a subject, the method comprising administering an effective amount of a composition according to any aspects, embodiments, or examples thereof as described herein. In some embodiments, there is provided a composition according to any aspects, embodiments, or examples thereof as described herein, for use in treating or preventing a disease, disorder, or condition, associated with ferroptosis of immune cells in a subject. In some embodiments, there is provided use of a composition according to any aspects, embodiments, or examples thereof as described herein, in the manufacture of a medicament for treating or preventing a disease, disorder, or condition, associated with ferroptosis of immune cells in a subject.

In some embodiments, there is provided a use of a composition according to any aspects, embodiments, or examples thereof as described herein for the manufacture of a medicament for improving immune health or an immune response in a subject in need thereof, or for treating or preventing an adverse response to a vaccination and/or viral infection in a subject.

In some embodiments, there is provided a use of a composition according to any aspects, embodiments, or examples thereof as described herein for the manufacture of a medicament for treating or preventing a disease, disorder, or condition, associated with ferroptosis of immune cells in a subject.

In some embodiments, there is provided a use of a composition according to any aspects, embodiments, or examples thereof as described herein the treatment of certain diseases disorder, or condition, associated with ferroptosis of immune cells where those diseases, disorder, or condition, which is associated with ferroptosis includes those such as cancer, inflammatory diseases including fatty liver disease, bacterial infections, pulmonary fibrosis, hepatitis, inflammatory bowel disease, neurodegenerative diseases, kidney injury, cardiovascular disease includings ischemia-reperfusion injury, skeletal muscle injury, neurodegeneration, Alzheimer's disease, and frailty in the elderly, which may be treated by blocking ferroptosis. Additional diseases which may benefit from treatment by blocking ferroptosis include sepsis, organ failure caused by septic shock, acute respiratory distress syndrome (ARDS), heart failure and cardiac injury.

In some embodiments, administering an effective amount of a composition as described in any embodiment herein results in a reduction in ferroptosis of immune cells. In some embodiments, the immune cells are selected from B-cells, T-cells, NK cells, monocytes, eosiniophils, or neutrophils. In some embodiments, the immune cells are T-cells. In some embodiments, the T-cells are follicular helper T (T_{FH}) cells.

In some embodiments, administering an effective amount of a composition as described in any embodiment herein results in treating or preventing a disease, disorder, or condition associated with the immune system in a subject. In some embodiments, the disease, disorder, or condition associated with the immune system in a subject is Alzheimer's disease, a viral infection, a metabolic disease, a cardiovascular disease, obesity, fatty liver disease, an inflammatory condition, or dyslipidaemia. In some embodiments, the disease, disorder, or condition associated with the immune system in a subject is a viral infection. In some embodiments, the viral infection is COVID-19. In some embodiments, the disease, disorder, or condition associated with the immune system in a subject is a metabolic disease. In some embodiments, the metabolic disease is diabetes. In some embodiments, the diabetes is type 1 diabetes or type 2 diabetes. In some embodiments, the disease, disorder, or condition associated with the immune system in a subject is an inflammatory condition. In some embodiments, the inflammatory condition is asthma or atopic dermatitis. In some embodiments, the inflammatory condition is associated with an increase in the levels of inflammatory cytokines. In some embodiments, the increase in the levels of inflammatory cytokines is an increase in the levels of TNFα.

In some embodiments, the treating or preventing a disease, disorder, or condition, associated with ferroptosis of immune cells in a subject comprises a reduction in oxidative cell death. In some embodiments, the treating or preventing a disease, disorder, or condition, associated with ferroptosis of immune cells in a subject comprises a reduction in the progression and symptoms associated with tumors, inflammatory diseases, bacterial infections, pulmonary fibrosis, hepatitis, inflammatory bowel disease, neurodegenerative diseases, kidney injury, ischemia-reperfusion injury, skeletal muscle injury, neurodegeneration and frailty in the elderly.

Any suitable administration regime may be followed. Administration of the composition, formulation, and/or product may be on a daily, twice to about 10× daily, weekly, bi-weekly, three weekly, monthly or ad hoc basis depending upon the subject, and for example the composition formulation, and/or product employed.

In the case of supplementation of infant formula, the composition may be provided as a component of infant formula and administered for example as part of the normal daily diet.

The production of the maintenance or modulatory compositions may for example comprise mixing the two or more ether lipid as described herein with a pharmaceutically or physiologically acceptable carrier.

The terms “effective amount” including “therapeutically effective amount” and “prophylactically effective amount” or “physiologically effective amount” as used herein mean a sufficient amount of a composition of the present application either in a single dose or as part of a series or slow release system which provides the desired therapeutic, preventative, or physiological effect in some subjects. Undesirable effects, e.g. side effects, may sometimes manifest along with the desired therapeutic effect; hence, a practitioner balances the potential benefits against the potential risks in determining an appropriate “effective amount”. The exact amount of composition required will vary from subject to subject, depending on the species, age and general condition of the subject, mode of administration and the like. Thus, it may not be possible to specify an exact ‘effective amount’. However, an appropriate ‘effective amount’ in any individual case may be determined by one of ordinary skill in the art using routine skills or experimentation. One of ordinary skill in the art would be able to determine the required amounts based on such factors as prior administration of the compositions or other agents, the subject's size, the severity of a subject's symptoms or the severity of symptoms in a population, and the particular composition or route of administration selected.

The term “treating” or “treatment”, for example in relation to immune health, metabolic disease, such as obesity or diabetes, or dyslipidemia refers to any measurable or statistically significant amelioration of metabolic disease, such as diabetes, obesity, or dyslipidemia. This can be assessed by measuring the herein defined ether lipid profile of the subject before and after administration. As used herein, the use of the terms “treating” and “treatment” in relation to a condition, disease or disorder, may include reducing the severity of the condition, disease or disorder, or reducing the severity and/or frequency of one or more symptoms of the condition, disease or disorder. As used herein, the term “treating” includes any effect, e.g., lessening, reducing, increasing, maintaining, modulating, ameliorating or eliminating, that results in the improvement of the condition, disease, disorder, and the like, or ameliorating a symptom thereof.

The terms “prevention” or “prophylaxis” relates to maintaining the in vivo defined ether lipid profile at or substantially the same as the non-disease profile identified herein. This can be assessed by periodically measuring the herein defined ether lipid profile of the subject. As used herein, the use of the terms “prevention” and “preventing” in relation to a condition, disease or disorder, may include reducing the likelihood that a subject will develop such a condition disease or disorder.

The present application provides methods of maintaining an in vivo defined ether lipid profile at or substantially the same as a reference non-disease profile identified herein by periodic supplementation of the composition or products as defined herein.

A “pharmacologically acceptable” composition is one tolerated by a recipient subject. It is contemplated that an effective amount of the composition is administered. An “effective amount” is an amount sufficient to achieve a desired biological effect such as to maintain, increase or modulate an ether lipid molecule profile in the subject for a period of time. Monitoring may by any convenient method known in the art. The actual effective amount may be dependent upon the type of subject/species their age, sex, health, and weight. Examples of desired biological effects include maintaining, increasing or modulating two or more ether lipid or plasmalogen species towards their healthy level as determined herein, or reducing the level of one or more ether lipid or plasmalogen species determined herein to be risk factors for immune health, ferroptosis, metabolic disease, diabetes, and their sequelae. In some embodiments, physiologically significant changes may only be achieved after a course of treatment in a proportion of suitable subjects.

The compositions of the present application can be administered as the sole active pharmaceutical agent, or used in combination with one or more agents to maintain, increase or beneficially modulate ether lipid molecule profiles in a subject. Profiles are readily determined using the protocols described herein.

The present disclosure also encompasses compositions, particularly pharmaceutical compositions, comprising the composition as defined herein together with a pharmaceutically acceptable carrier and/or diluent.

As used herein, the term “composition” refers to a product comprising a particular ingredient in a particular amount and any product directly or indirectly brought about by the combination of particular ingredients in particular amounts. In some embodiments, a composition comprises an active ingredient and an inactive ingredient. In some embodiments, the composition is a formulation. In some embodiments, the formulation is a composition that is suitable for administration to and/or consumption by a subject, such as a human. A composition may be a pharmaceutical composition, and a formulation may be a pharmaceutical formulation. In some embodiments, the pharmaceutical composition or pharmaceutical formulation comprises a combination of an active agent with a pharmaceutically acceptable carrier, diluent, excipient, solubilizing agent, or vehicle, inert or active. As used herein, the term “pharmaceutically acceptable” means that a carrier, diluent, excipient, solubilizing agent, or vehicle is compatible with other components of a formulation and is nontoxic to a subject. Thus, it is to be understood that a “pharmaceutical composition” or a “pharmaceutical formulation” is appropriate for administration to and/or consumption by a subject, such as a human, and may, for example, be approved by the U.S. Food and Drug Administration and/or the European Medicines Agency for such administration and/or consumption. It is also to be understood that a composition or formulation may not necessarily be, for example, approved by the U.S. Food and Drug Administration and/or the European Medicines Agency for administration to and/or consumption by a subject, such as a human.

Pharmaceutical compositions include a product comprising an active ingredient and an inert ingredient constituting a carrier and include every product directly or indirectly brought about by the combination, complexation or aggregation of any two or more ingredients or the dissociation, other kinds of reactions or interaction of one or more ingredients. Thus, the pharmaceutical composition of the present disclosure includes every composition prepared by mixing the at least one compound of the present disclosure with a pharmaceutically acceptable carrier, diluent, excipient, solubilizing agent, or vehicle.

In some embodiments, a composition and/or formulation of the disclosure is in the form of a beverage or a food product. In some embodiments, the beverage or food product is formulated for general consumption, such as by being food grade. In some embodiments, the beverage or food product is formulated as a dietary supplement or other nutritional composition. In some embodiments, the beverage or food product is pharmaceutical grade. For the avoidance of doubt, it is to be understood that, in some embodiments, a composition and/or formulation of the disclosure may be suitable for consumption by a subject, such as a human, but not necessarily be of pharmaceutical grade (for example, by being of food grade or nutritional composition grade, but not necessarily pharmaceutical grade).

In some embodiments, the composition and/or formulation may contain one or more of a solubilizing agent, an emulsifier, a stabilizer, a dispersal agent, an anti-foaming agent, or a diluent. The composition and/or formulation may additionally contain one or more antioxidant compounds. It is anticipated that any antioxidants suitable for oral administration, such as vitamin A, vitamin E, vitamin C, retinal, tocopherol, L-ascorbyl palmitate, riboflavin and carotenoids, including lutein, beta-carotene, zeaxanthin, and lycopene, and combinations thereof, may be formulated into an embodiment described herein. In some embodiments, the composition is naturally unreactive to oxidation and does not require the addition of antioxidant compounds.

As used herein, the term “excipient” shall mean an inactive ingredient used as a vehicle (e.g., water, capsule shell, etc.), a diluent, or a component to constitute a dosage form or pharmaceutical composition comprising a drug such as a therapeutic agent. The term also encompasses an inactive ingredient that imparts cohesive function (e.g., binder), disintegrating function (e.g., disintegrator), lubricant function (e.g., lubricating agent), and/or the other function (e.g., solvent, surfactant, etc.) to the composition.

As used herein, the term “pharmaceutically acceptable salt” refers to any pharmaceutically acceptable salt (e.g., acid or base) of a compound of the present disclosure which, upon administration to a subject, is capable of providing a compound of this disclosure or an active metabolite or residue thereof. For example, Berge et al. describe pharmaceutically acceptable salts in detail in J. Pharmaceutical Sciences (1977) 66:1-19. As is known to those of skill in the art, “salts” of the compounds of the present disclosure may be derived from inorganic or organic acids and bases. The salts can be prepared in situ during the final isolation and purification of the compounds of the disclosure or separately by reacting a free base function with a suitable acid. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids while not in themselves pharmaceutically acceptable, may be employed in the preparation of salts useful as intermediates in obtaining the compounds of the disclosure and their pharmaceutically acceptable acid addition salts. Examples of bases include, but are not limited to, alkali metals (e.g., sodium) hydroxides, alkaline earth metals (e.g., magnesium), hydroxides, ammonia, and compounds of formula NW4+, wherein each W is independently selected from H or C₁₋₄alkyl, and the like. Basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain alkyl halides such as decyl, lauryl, myristyl, and stearyl chlorides, bromides, and iodides; arylalkyl halides such as benzyl and phenethyl bromides; and others. Products having modified solubility or dispersibility are thereby obtained.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present disclosure compounded with a suitable cation such as Na⁺, NH4⁺, and NW4⁺ (wherein each W is independently selected from H or C₁₋₄alkyl,), and the like. For therapeutic use, salts of the compounds of the present disclosure are contemplated as being pharmaceutically acceptable. However, salts of acids and bases that are non-pharmaceutically acceptable may also find use, for example, in the preparation or purification of a pharmaceutically acceptable compound.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see REMINGTON'S PHARMACEUTICAL SCIENCES (Mack Publishing Co., Easton, PA).

As used herein, in some embodiments, the term “carrier” refers to a pharmaceutically acceptable carrier. In some embodiments, the term “carrier” refers to any suitable carrier, which may not be of pharmaceutical grade. For example, in some embodiments, the carrier is of food grade but may not be of pharmaceutical grade.

The term “day” as used herein is taken to mean a 24 hour period of time.

As a general matter, compositions specifying a percentage are by weight unless otherwise specified.

A pharmaceutical composition may comprise the ether lipid mixture as described herein, in combination with a standard, well-known, non-toxic pharmaceutically-acceptable carrier, adjuvant or vehicle such as phosphate-buffered saline, water, ethanol, polyols, vegetable oils, a wetting agent or an emulsion such as a water/oil emulsion. The composition may be in either a liquid or solid form. For example, the composition may be in the form of a tablet, capsule, ingestible liquid, spray, or powder, injectable, or topical ointment or cream. Proper fluidity can be maintained, for example, by the maintenance of the required particle size in the case of dispersions and by the use of surfactants. It may also be desirable to include isotonic agents, for example, sugars, sodium chloride, and the like. Besides such inert diluents, the composition can also include adjuvants, such as wetting agents, emulsifying and suspending agents, sweetening agents, flavouring agents and perfuming agents.

Suspensions, in addition to the active compounds, may comprise suspending agents such as ethoxylated isostearyl alcohols, polyoxyethylene sorbitol and sorbitan esters, microcrystalline cellulose, aluminum metahydroxide, bentonite, agar-agar, and tragacanth or mixtures of these substances.

Solid dosage forms such as tablets and capsules can be prepared using techniques well known in the art. For example, ether lipid mixtures produced in accordance with the present disclosure can be tableted with conventional tablet bases such as lactose, sucrose, and cornstarch in combination with binders such as acacia, cornstarch or gelatin, disintegrating agents such as potato starch or alginic acid, and a lubricant such as stearic acid or magnesium stearate. Capsules can be prepared by incorporating these excipients into a gelatin capsule along with antioxidants and the relevant fatty acid(s).

For intravenous administration, the composition may be incorporated into commercial compositions and/or formulations. Examples of pharmaceutically acceptable carriers and methods of manufacture of multiple composition formats may be found in the most recent edition of Remington's Pharmaceutical Sciences, Mack Publishing, Easton.

A typical dosage of a composition as described herein is from 0.1 mg to 20 g, taken from one to five times per day and is preferably in the range of from about 10 mg to about 1, 2, 5, or 10 g daily (taken in one or multiple doses). Non-limiting illustrative doses of a composition as described in the present application are 100 to 3000 mg once or twice daily. In another example the composition is added to an oral product and administered at a percent by weight of 0.01% to 10% of the product.

Dosages and serving sizes may be varied as appropriate to age, height, and weight of the subject. In an embodiment, a useful human dose may be from 2 mg/kg to 3 mg/kg, which may amount to from 100 mg to 300 mg per day. In a further embodiment, a useful human dose may be 200 mg per day.

In another embodiment, a maintenance dose may be from 0.2 mg/kg to 2 mg/kg or from 25 mg to 100 mg per day. That is, in some embodiments, a maintenance dose may be 25 mg, 50 mg, or 100 mg per day.

In another embodiment, where a large elevation of plasmalogens or a rapid elevation thereof is desirable, a dose may consist of from 3 mg/kg to 25 mg/kg and/or 300 mg to 2000 mg per day. That is, where a large elevation of plasmalogens or a rapid elevation thereof is desirable, a dose may consist of 400 mg, 800 mg or 1600 mg per day. In another embodiment, a dose may consist of 0.1 mg per day to 4,000 mg per day. In another embodiment, a dose may consist of from 0.1 mg per day to 2,000 mg per day. Possible routes of administration of the pharmaceutical compositions of the presently described compositions include, for example, enteral (e.g., oral and rectal) and parenteral. For example, a liquid preparation may be administered orally or rectally. Additionally, a homogenous mixture can be completely dispersed in water, admixed under sterile conditions with physiologically acceptable diluents, preservatives, buffers or propellants to form a spray or inhalant.

The dosage of the composition to be administered to the subject may be determined by one of ordinary skill in the art and depends upon various factors such as weight of the subject, age and species of the subject, overall health of the subject, past history of the subject, immune status of the patient, etc.

Additionally, the compositions of the present disclosure may be utilized for cosmetic purposes. It may be added to pre-existing cosmetic compositions such that a mixture is formed and may be used as the sole “active” ingredient in a cosmetic composition.

### Delivery System

The compositions and/or formulations disclosed herein may be delivered via dosage forms including, but not limited to, tablets, capsules, solutions, suspensions, powders, gums, and confectionaries. The compositions and/or formulations disclosed herein may be delivered via sublingual delivery systems including, but not limited to, dissolvable tabs under and on the tongue, liquid drops, and beverages. Alternatively, or in addition, edible films, hydrophilic polymers, oral dissolvable films, or oral dissolvable strips can be used.

For oral administration, the compositions and/or formulations disclosed herein may be further combined with one or more solid inactive ingredients for the preparation of tablets, capsules, pills, powders, granules, or other suitable dosage forms. For example, the composition and/or formulation components may be combined with at least one excipient including, but not limited to, those selected from the group consisting of fillers, binders, humectants, disintegrating agents, solution retarders, absorption accelerators, wetting agents, absorbents, and lubricating agents. Other useful excipients include, but are not limited to, magnesium stearate, calcium stearate, mannitol, xylitol, sweeteners, starch, carboxymethylcellulose, microcrystalline cellulose, silica, gelatin, silicon dioxide, and the like. In some embodiments, compositions and/or formulations according to the disclosure may include one or more of beeswax (such as beeswax E901), carnauba wax (such as carnauba wax E903), shellac (such as shellac E904), candelilla wax (such as candelilla wax E902), microcrystalline wax (such as microcrystalline wax E905), paraffin wax, and di-acylglycerols.

The components of the compositions and/or formulations administered according to the methods of the present disclosure can be administered in a wide variety of oral dosage forms. It will be obvious to those skilled in the art that suitable dosage forms may comprise, in certain embodiments one or more chemical compounds of the present disclosure and/or one or more pharmaceutically acceptable salts of a chemical compound of the present disclosure.

For preparing pharmaceutical formulations or compositions to be administered according to the methods of the present disclosure, pharmaceutically acceptable carriers can be either solid or liquid. Solid form preparations include powders, tablets, pills, capsules, and cachets. A solid carrier can be one or more substances that may also act as diluents, flavoring agents, solubilizing agents, lubricants, suspending agents, binders, preservatives, tablet disintegrating agents, or encapsulating materials.

In powders, the carrier is a finely divided solid, which is in a mixture with the finely divided active component. In tablets, one or more compounds of the present disclosure and/or pharmaceutically acceptable salts thereof is mixed with one or more carriers having the necessary binding capacity in suitable proportions, which is then compacted in the shape and size desired.

In certain embodiments, powders and tablets administered according to methods of the present disclosure preferably may contain, in total, from about one to about ninety-nine percent, such as from five or ten to about seventy percent one or more compounds of the present disclosure and/or pharmaceutically acceptable salts thereof. Suitable carriers include, but are not limited to, are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of one or more compounds of the present disclosure and/or pharmaceutically acceptable salts thereof with encapsulating material as a carrier providing a capsule in which one or more compounds of the present disclosure and/or pharmaceutically acceptable salts thereof, with or without additional carriers, is surrounded by a carrier, which is thus in association with it. Similarly, cachets and lozenges are included. Tablets, powders, capsules, pills, cachets, and lozenges are included. Tablets, powders, capsules, pills, cachets, lozenges and rapid melts can be used as solid forms suitable for oral administration.

Capsules may be prepared in such a way as to be additionally coated for timed release. Coating thickness may be modified to provide a delayed release of the capsule contents. Capsules may be prepared in such a way as to be targeted release capsules. In some embodiments, the capsule can be targeted to the stomach. In some embodiments, the capsule targeted for delivery to the stomach is coated in a film coating. In some embodiments, the capsule can be targeted to the small intestine. In some embodiments, the capsule targeted for delivery to the small intestine is coated in an enteric coating.

The term “rapid-melt” as used herein refers to pharmaceutical formulations or compositions that melt on contact with saliva requiring little or no chewing.

Liquid preparations include, but are not limited to, solutions, suspensions, and emulsions, for example, water or water-propylene glycol solutions. The formulated preparations may take such forms as suspensions, solutions, or emulsions in oily or aqueous vehicles, and may contain composition and/or formulation agents such as suspending, stabilizing, solubilizing, and/or dispersing agents. Alternatively, one or more compounds of the present disclosure and/or pharmaceutically acceptable salts thereof may be in powder form, such as that obtained by aseptic isolation of sterile solid or by lyophilization from solution, for constitution with a suitable vehicle, e.g., sterile, pyrogen-free water, before use.

Aqueous solutions suitable for oral use can be prepared by dissolving one or more compounds of the present disclosure, and/or pharmaceutically acceptable salts thereof, in water, and adding suitable colorants, flavors, stabilizing and thickening agents, as desired. Aqueous suspensions suitable for oral use can be made by solubilizing, and/or dispersing the finely divided active component in water with viscous material, such as natural or synthetic gums, resins, methylcellulose, sodium carboxymethylcellulose, or other well-known suspending agents.

Compositions and/or formulations suitable for topical administration in the mouth, or buccal, or sublingual administration include, but are not limited to: lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

In some embodiments, a composition and/or formulation comprises a solubilizing agent. As used herein, the term “solubilizing agent” refers to any agent which promotes solubilization or dispersal of the composition when placed into a liquid. In some embodiments, a “solubilizing agent” may be a dispersal agent. The solubilizing agent may additionally improve the stability of the formulated composition. Suitable solubilizing agents include, but are not limited to, carboxymethylcellulose sodium, hypromellose, proline, xanthan gum, maltodextrin, alginates, waxes, lipids, oils, alcohols, sugars, microcrystalline cellulose, starches, calcium phosphates, mannitol, sorbitol, erythritol, food grade solvents, phospholipids, such as lecithin (including, but not limited to, egg yolk L-α-lecithin, such as egg yolk L-α-lecithin available from Sigma-Aldrich, Saint Louis, M.O., U.S.A.), DMSO, ethanol, ethyl acetate, isopropanol, and the like. In some embodiments, a solubilizing agent is used to improve the separation of the compounds which make up the composition and/or formulation and to prevent their settling or clumping in compositions and/or formulations. In some embodiments, a composition and/or formulation comprises a solubilizing agent and a suitable carrier. In some embodiments, such a composition and/or formulation is a pharmaceutical-grade product. In some embodiments, such a composition and/or formulation is a food-grade product.

The pharmaceutical compositions and/or formulations and/or preparations are preferably in unit dosage forms. In such form, the composition and/or formulation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself; or it can be the appropriate number of any of these in packaged form.

Tablets, capsules, and lozenges for oral administration and liquids for oral use are preferred compositions and/or formulations.

Further details on techniques for formulation and administration may be found in the latest edition of REMINGTON'S PHARMACEUTICAL SCIENCES (Mack Publishing Co., Easton, PA).

Illustrative methods capable of analysing lipid species include classical lipid extraction methods, mass spectrometry together with electrospray ionization and matrix-assisted laser desorption ionisation, with mass analysis such as quadruple and/or TOF (e.g. Quadrapole/TOF) or orbitrap mass analysers. Chromatographic methods are used for the separation of lipid mixtures such as gas chromatography, high pressure liquid chromatography (HPLC), ultra-high pressure liquid chromatography (UHPLC), capillary electrophoresis (CE). These may be used with mass spectrometry based detection systems or other detectors including optical detectors. Clinical mass spectrometry systems are used by clinical laboratories to provide lipid profiles and ratios upon request. Another suitable technique for quantitative lipid analysis is one or two dimensional nuclear magnetic resonance (NMR). Two dimensional techniques such as heteronuclear single quantum coherence (HSQC) are suitable for lipid profiling through the ability to elucidate C—H bonds within a structure. Any technique capable of identifying individual lipid species in the sample can be used for collecting information on the lipid species. Typically, MS is used coupled to a separation method such as various forms of chromatography.

### NUMBERED EMBODIMENTS

1. A composition for increasing in vivo ether lipids in immune cells comprising a mixture of two or more ether lipid molecules of Compound 1:

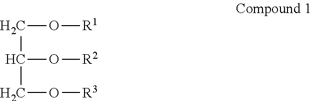

wherein
R¹ is an alkyl or alkenyl group;

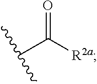

R² is hydrogen or
R³ is hydrogen,

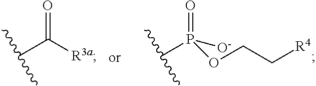

R^{2a} and R^{3a} are each an alkyl or alkenyl group; and
R⁴ is —N(Me)₃⁺ or —NH₃⁺.
2. The composition according to embodiment 1, wherein the immune cells are selected from B-cells, T-cells, NK cells, monocytes, eosiniophils, or neutrophils.
3. The composition according to embodiment 1 or embodiment 2, wherein the composition is for increasing in vivo ether lipids in immune cells at coordinated ratios associated with a non-disease state of the immune cells.
4. The composition according to any one of embodiments 1 to 3, wherein the increase of ether lipids in the immune cells (in %) is at least about 5, 10, 15, 20, 25, 30, 35, 40, 45, 50, 60, 70, 80, 90, 100, 110, 120, 130, 140, 150, 160, 170, 180, 190, or 200.
5. The composition according to any one of embodiments 1 to 4, wherein the ether lipids increased in the immune cells are plasmanyl- and/or plasmenyl-phospholipids.
6. The composition according to any one of embodiments 1 to 5, wherein the ether lipids increased in the immune cells are plasmalogens.
7. The composition according to any one of embodiments 1 to 6, wherein the ether lipids increased in the immune cells comprise at least two ether lipids of Compound 1 wherein R¹ is selected from C₁₆alkyl, C₁₆alkenyl, C₁₈alkyl, and C₁₈alkenyl.
8. The composition according to any one of embodiments 1 to 7, wherein the composition comprises at least two ether lipid molecules of Compound 1 wherein R¹ is selected from a C₁₆alkyl, C₁₆alkenyl, C₁₈alkyl, and C₁₈alkenyl.
9. The composition according to any one of embodiments 1 to 8, wherein the composition comprises at least two ether lipid molecules of Compound 1 wherein R¹ is selected from 16:0 alkyl, 18:0 alkyl, and 18:1 alkenyl.
10. The composition according to any one of embodiments 1 to 9, wherein the composition comprises an ether lipid molecule of Compound 1 wherein R¹ is 16:0 alkyl and an ether lipid molecule of Compound 1 wherein R¹ is 18:0 alkyl.
11. The composition according to embodiment 10, wherein the ratio of the 16:0 alkyl ether lipid molecules to the 18:0 alkyl ether lipid molecules is in a range of about 0.85:1 to 3:1, 1:1 to 2.5:1, or 1.5:1 to 2:1.
12. The composition according to embodiment 10, wherein the composition has a molar percent of 16:0 alkyl ether lipid molecules in the range of from 35% to 80%, 40% to 70%, or 45% 65%, and a molar percent of 18:0 alkyl ether lipid molecules in the range of from 15% to 60%, 20% to 50%, or 25% to 45%.
13. The composition according to any one of embodiments 1 to 12, wherein the composition comprises an ether lipid molecule of Compound 1 wherein R¹ is 16:0 alkyl and an ether lipid molecule of Compound 1 wherein R¹ is 18:1 alkenyl.
14. The composition according to embodiment 13, wherein the ratio of the 16:0 alkyl ether lipid molecule to the 18:1 alkenyl ether lipid molecule is in a range of about 2:1 to 10:1, 3:1 to 7:1, or 4:1 to 6:1.
15. The composition according to embodiment 13, wherein the composition has a molar percent of 16:0 alkyl ether lipid molecules in the range of from 35% to 80%, 40% to 70%, or 45% 65%, and a molar percent of 18:1 alkenyl ether lipid molecules in the range of from about 1% to 20%, 2% to 18%, or 5% to 15%.
16. The composition according to any one of embodiments 1 to 15, wherein the composition comprises an ether lipid molecule of Compound 1 wherein R¹ is 18:0 alkyl and an ether lipid molecule of Compound 1 wherein R¹ is 18:1 alkenyl.
17. The composition according to embodiment 16, wherein the ratio of the 18:0 alkyl ether lipid molecule to the 18:1 alkenyl ether lipid molecule is in a range of about 1.5:1 to 10:1, 2:1 to 7:1, or 3:1 to 6:1.
18. The composition according to embodiment 17, wherein the composition has a molar percent of 18:0 alkyl ether lipid molecules in the range of from 15% to 60%, 20% to 50%, or 25% to 45%, and a molar percent of 18:1 alkenyl ether lipid molecules in the range of from about 1% to 20%, 2% to 18%, or 5% to 15%.
19. The composition according to any one of embodiments 1 to 18, wherein the composition comprises an ether lipid molecule of Compound 1 wherein R¹ is 16:0 alkyl, an ether lipid molecule of Compound 1 wherein R¹ is 18:0 alkyl, and an ether lipid molecule of Compound 1 wherein R¹ is 18:1 alkenyl.
20. The composition according to embodiment 19, wherein the composition has a molar percent of 16:0 alkyl ether lipid molecules in the range of from 35% to 80%, 40% to 70%, or 45% 65%, a molar percent of 18:0 alkyl ether lipid molecules in the range of from 15% to 60%, 20% to 50%, or 25% to 45%, and a molar percent of 18:1 alkenyl ether lipid molecules in the range of from about 1% to 20%, 2% to 18%, or 5% to 15%.
21. The composition according to any one of embodiments 1 to 20, wherein the ether lipids having an 18:1 alkenyl R¹ group, ether lipids having an 18:0 alkyl R¹ group, and/or ether lipids having a 16:0 alkyl R¹ group, if present, together comprise at least about 10, 20, 30, 40, 50, 60, 70, 80, or 90% of total ether lipids in the composition.
22. A composition according to any one of embodiments 1 to 21, wherein the composition is in the form of a product which is a dietary supplement, capsule, syrup, liquid, food or beverage.
23. The composition according to any one of embodiments 1 to 22, wherein the composition is prepared by mixing a plurality of ether lipids in ratios and/or levels corresponding with ratios and/or levels associated with a non-disease state in vivo immune cell.
24. A composition for increasing in vivo ether lipids in immune cells comprising a mixture of two or more ether lipid molecules of Compound 1A:

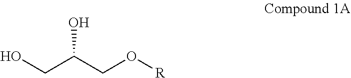

wherein
R is an alkyl or alkenyl group;
wherein the alkyl or alkenyl group is a C₁-C₃₀ alkyl, or C₁-C₃₀ alkenyl chain.
25. The composition according to embodiment 24, wherein the immune cells are selected from B-cells, T-cells, NK cells, monocytes, eosiniophils, or neutrophils.
26. The composition according to embodiment 25, wherein the immune cells are T-cells.
27. The composition according to embodiment 25, wherein the immune cells are monocytes.
28. The composition according to any one of embodiments 24 to 27, wherein the composition is for increasing in vivo ether lipids in immune cells at coordinated ratios associated with a non-disease state of the immune cells.
29. The composition according to any one of embodiments 24 to 28, wherein the increase of ether lipids in the immune cells (in %) is at least about 5, 10, 15, 20, 25, 30, 35, 40, 45, 50, 60, 70, 80, 90, 100, 110, 120, 130, 140, 150, 160, 170, 180, 190, or 200.
30. The composition according to any one of embodiments 24 to 29, wherein the ether lipids increased in the immune cells are plasmanyl- and/or plasmenyl-phospholipids.
31. The composition according to any one of embodiments 24 to 30, wherein the ether lipids increased in the immune cells are plasmalogens.
32. The composition according to any one of embodiments 24 to 31, wherein the ether lipids increased in the immune cells comprise at least two ether lipids of Compound 1A wherein R is selected from C₁₆alkyl, C₁₆alkenyl, C₁₈alkyl, and C₁₈alkenyl.
33. The composition according to any one of embodiments 24 to 32, wherein the mixture of two or more ether lipid molecules of Compound 1A comprises two or more ether lipid molecules selected from chimyl alcohol, batyl alcohol and selachyl alcohol.
34. The composition of any one of embodiments 24 to 33, wherein the mixture of two or more ether lipids comprises chimyl alcohol.
35. The composition of any one of embodiments 24 to 34, wherein the mixture of two or more ether lipids comprises chimyl alcohol in a molar percent amount of 19-55% of the total ether lipids or comprises chimyl alcohol in a weight percent amount of 19-55% of the total ether lipids.
36. The composition of any one of embodiments 24 to 35, wherein the mixture of two or more ether lipids comprises batyl alcohol.
37. The composition of any one of embodiments 24 to 36, wherein the mixture of two or more ether lipids comprises batyl alcohol in a molar percent amount of 5-35% of the total ether lipids or comprises batyl alcohol in a weight percent amount of 5-35% of the total ether lipids.
38. The composition of any one of embodiments 24 to 37, wherein the mixture of two or more ether lipids comprises selachyl alcohol.
39. The composition of any one of embodiments 24 to 38, wherein the mixture of two or more ether lipids comprises selachyl alcohol in a molar percent amount of 10-76% of the total ether lipids or comprises selachyl alcohol in a weight percent amount of 10-76% of the total ether lipids.
40. The composition of embodiment 33, wherein the mixture of two or more ether lipid molecules of Compound 1A comprises a mixture of chimyl alcohol and batyl alcohol.
41. The composition of embodiment 33 or embodiment 40, wherein the mixture of two or more ether lipids comprises a mixture of chimyl alcohol in a molar percent amount of 19-55% of the total ether lipids and batyl alcohol in a molar percent amount of 5-35% of the total ether lipids or comprises a mixture of chimyl alcohol in a weight percent amount of 19-55% of the total ether lipids and batyl alcohol in a weight percent amount of 5-35% of the total ether lipids.
42. The composition of embodiment 33, wherein the mixture of two or more ether lipid molecules of Compound 1A comprises a mixture of chimyl alcohol and selachyl alcohol.
43. The composition of embodiment 33 or embodiment 42, wherein the mixture of two or more ether lipids comprises a mixture of chimyl alcohol in a molar percent amount of 19-55% of the total ether lipids and selachyl alcohol in a molar percent amount of 10-76% of the total ether lipids or comprises a mixture of chimyl alcohol in a weight percent amount of 19-55% of the total ether lipids and selachyl alcohol in a weight percent amount of 10-76% of the total ether lipids.
44. The composition of embodiment 33, wherein the mixture of two or more ether lipid molecules of Compound 1A comprises a mixture of batyl alcohol and selachyl alcohol.
45. The composition of embodiment 33 or embodiment 44, wherein the mixture of two or more ether lipids comprises a mixture of batyl alcohol in a molar percent amount of 5-35% of the total ether lipids and selachyl alcohol in a molar percent amount of 10-76% of the total ether lipids or comprises a mixture of batyl alcohol in a weight percent amount of 5-35% of the total ether lipids and selachyl alcohol in a weight percent amount of 10-76% of the total ether lipids.
46. The composition of embodiment 33, wherein the mixture of two or more ether lipid molecules of Compound 1A comprises chimyl alcohol, batyl alcohol and selachyl alcohol.
47. The composition of embodiment 46, wherein the chimyl alcohol is present in a molar percent amount of 19% of the total ether lipids, batyl alcohol is present in a molar percent amount of 5% of the total ether lipids, and selachyl alcohol is present in a molar percent amount of 76% of the total ether lipids.
48. The composition of embodiment 46, wherein the chimyl alcohol is present in an amount of 50% of the total ether lipids by weight, batyl alcohol is present in an amount of 30% of the total ether lipids by weight, and selachyl alcohol is present in an amount of 20% of the total ether lipids by weight.
49. The composition of embodiment 46, wherein the chimyl alcohol is present in a molar percent amount of 55% of the total ether lipids, batyl alcohol is present in a molar percent amount of 35% of the total ether lipids, and selachyl alcohol is present in a molar percent amount of 10% of the total ether lipids.
50. The composition according to any one of embodiments 24 to 49, wherein the composition is formulated for oral administration.
51. The composition according to any one of embodiments 24 to 50, wherein the composition is a tablet, a capsule, a solution, a mouthwash, a suspension, a syrup, a powder, a gum, a food product, a beverage, a dietary supplement, a confectionary, a lozenge, a sublingual delivery system, or a rapid-melt formulation.
52. The composition according any one of embodiments 24 to 51, wherein the composition is a food product.
53. The composition according any one of embodiments 24 to 51, wherein the composition is a dietary supplement.
54. The composition according any one of embodiments 24 to 52, wherein the food product is a medical food product.
55. The composition according any one of embodiments 24 to 52, wherein the food product is infant formula.
56. The composition according to any one of embodiments 24 to 55, wherein the composition is prepared by mixing a plurality of ether lipids in ratios and/or levels corresponding with ratios and/or levels associated with a non-disease state in vivo immune cell.
57. A composition of any one of embodiments 1 to 56 for use in therapy.
58. A nutritional composition comprising a composition of any one of embodiments 1 to 57 and a potable excipient.
59. A pharmaceutical composition comprising a composition of any one of embodiments 1 to 57 and a pharmaceutically acceptable excipient.
60. A method of increasing in vivo ether lipids in immune cells comprising administering to a subject an effective amount of a composition according to any one of embodiments 1 to 59.
61. The method according to embodiment 60, wherein the immune cells are selected from B-cells, T-cells, NK cells, monocytes, eosiniophils, and/or neutrophils.
62. The method according to embodiment 60 or embodiment 61, wherein the method is for increasing in vivo ether lipids in immune cells at coordinated ratios associated with a non-disease state of the immune cells.
63. The method according to any one of embodiments 60 to 62, wherein the increase of ether lipids in the immune cells (in %) of the subject is at least about 5, 10, 15, 20, 25, 30, 35, 40, 45, 50, 60, 70, 80, 90, 100, 110, 120, 130, 140, 150, 160, 170, 180, 190, or 200.
64. The method according to any one of embodiments 60 to 63, wherein the ether lipids increased in the immune cells are plasmanyl- and/or plasmenyl-phospholipids.
65. The method according to any one of embodiments 60 to 64, wherein the ether lipids increased in the immune cells are plasmalogens.
66. The method according to any one of embodiments 60 to 65, wherein the ether lipids increased in the immune cells comprise at least two ether lipids of Compound 1 wherein R¹ is selected from C₁₆alkyl, C₁₆alkenyl, C₁₈alkyl, and C₁₈alkenyl.
67. The method according to any one of embodiments 60 to 66, wherein the ether lipids increased in the immune cells are in coordinated ratios corresponding to the ether lipid molecules as defined in any one of claims 1 to 56.
68. The method according to any one of embodiments 60 to 67, further comprising (i) identifying a subject with low amount of plasmalogen content in their immune cells, and (ii) administering to the subject a composition as defined in any one of claims 1 to 56 having coordinated ratios of ether lipid molecules that substantially correspond to the plasmalogen profile in the immune cells to elevate two or more key plasmalogen species in a coordinated manner to increase the level of plasmalogens in the immune cells.
69. The method according to embodiment 68, wherein the total plasmalogen content in the immune cells of a subject prior to treatment is less than about 10, 9, 8, 7, 6, 5, 4, 3, or 2 molar % of total lipids.
70. A method of treating or preventing a disease, disorder, or condition, associated with the immune system in a subject, the method comprising administering an effective amount of a composition according to any one of embodiments 1 to 59 to the subject.
71. A method of improving immune health or an immune response in a subject, the method comprising administering an effective amount of a composition according to any one of embodiments 1 to 59 to the subject.
72. The method of embodiment 70 or embodiment 71, wherein the disease, disorder, or condition in a subject is cardiovascular disease, Alzheimer's disease, viral infection, metabolic disease, diabetes, cardiovascular disease, obesity, fatty liver disease, an inflammatory condition, or dyslipidemia.
73. A method of improving response to vaccination in a subject, the method comprising administering an effective amount of a composition according to any one of embodiments 1 to 59 to the subject.
74. A method of treating or preventing an adverse response to a viral infection in a subject, the method comprising administering an effective amount of a composition according to any one of embodiments 1 to 59 to the subject to provide an enhanced immune response in the subject.
75. A method of treating or preventing a disease, disorder, or condition associated with ferroptosis of immune cells in a subject, the method comprising administering an effective amount of a composition according to any one of embodiments 1 to 59 to the subject.
76. Use of a composition of any one of embodiments 1 to 59 for the manufacture of a medicament for improving immune health or an immune response in a subject in need thereof, or for treating or preventing an adverse response to a vaccination and/or viral infection in a subject.
77. Use of a composition of any of embodiments 1 to 59 for the manufacture of a medicament for treating or preventing a disease, disorder, or condition associated with ferroptosis of immune cells in a subject.
78. A method of reducing ferroptosis in immune cells comprising administering to a subject an effective amount of a composition according to any one of embodiments 1 to 59.
79. The method of embodiment 78, wherein the immune cells are selected from B-cells, T-cells, NK cells, monocytes, eosiniophils, or neutrophils.
80. The method of embodiment 79, wherein the immune cells are T-cells.
81. The method of embodiment 80, wherein the T-cells are follicular helper T (T_{FH}) cells.
82. A method of treating or preventing a disease, disorder, or condition associated with the immune system in a subject, the method comprising administering an effective amount of a composition according to any one of embodiments 1 to 59 to the subject.
83. The method of embodiment 82, wherein the disease, disorder, or condition associated with the immune system in a subject is Alzheimer's disease, a viral infection, a metabolic disease, a cardiovascular disease, obesity, fatty liver disease, an inflammatory condition, or dyslipidaemia.
84. The method of embodiment 82 or embodiment 83, wherein the disease, disorder, or condition associated with the immune system in a subject is a viral infection, a metabolic disease, or an inflammatory condition.
85. The method of embodiment 83 or embodiment 84, wherein the viral infection is COVID-19 infection.
86. The method of embodiment 83 or embodiment 84, wherein the metabolic disease is diabetes.
87. The method of embodiment 86, wherein the diabetes is type 2 diabetes.
88. The method of embodiment 83 or embodiment 84 wherein the inflammatory condition is asthma or atopic dermatitis.
89. The method of any one of embodiments 83, 84, or 88, wherein the inflammatory condition is associated with an increase in the levels of inflammatory cytokines.
90. The method of embodiment 89, wherein the increase in the levels of inflammatory cytokines is an increase in the levels of TNFα.
91. The method of embodiment 75 or embodiment 77, wherein the treating or preventing a disease, disorder, or condition associated with ferroptosis of immune cells in a subject comprises a reduction in oxidative cell death.
92. The method of embodiment 91, wherein the treating or preventing a disease, disorder, or condition associated with ferroptosis of immune cells in a subject comprises a reduction in ferroptosis.
93. The method of embodiment 91 or embodiment 92, wherein the treating or preventing a disease, disorder, or condition associated with ferroptosis of immune cells in a subject comprises a reduction in the progression and symptoms associated with tumors, inflammatory diseases, bacterial infections, pulmonary fibrosis, hepatitis, inflammatory bowel disease, neurodegenerative diseases, kidney injury, ischemia-reperfusion injury, skeletal muscle injury, neurodegeneration, Alzheimer's disease, and frailty in the elderly.
94. The method of any one of embodiments 60 to 93, wherein the composition of ether lipids is administered in a dose of from 0.1 mg per day to 4,000 mg per day.
95. The method of any one of embodiments 60 to 94, wherein the composition of ether lipids is administered in a dose of from 0.1 mg per day to 2,000 mg per day.
96. The method of any one of embodiments 60 to 95, wherein the composition of ether lipids is administered in a dose of from 25 mg per day to 1600 mg per day.
97. The method of any one of embodiments 60 to 96, wherein the composition of ether lipids is administered in a dose of 400 mg, 800 mg, or 1600 mg per day.
98. The method of any one of embodiments 60 to 96, wherein the composition of ether lipids is administered in a dose of 200 mg per day.
99. The method of any one of embodiments 60 to 96, wherein the composition of ether lipids is administered in a dose of from 25 mg per day to 100 mg per day.

### EXAMPLES

The present description is further illustrated by the following examples, which should not be construed as limiting in any way.

### Example 1: Plasmalogen Composition for Different Immune Cell Types

### Preparation of Human Immune Cells for Lipidomic Analysis

Human immune cells were obtained from buffy coats (collected from 9-14 individual donors) received from Red Cross Australia (Melbourne, Australia). Buffy coats were diluted 1:5 with PBS containing 5% FBS and 0.5 mM EDTA. Blood was layered onto a discontinuous Histopaque (Sigma Aldrich, NSW, Australia) density gradient with the densities 1.077 g/ml and 1.119 g/ml to isolate PBMCs and granulocytes, respectively, and centrifuged for 30 mins at 300 g with the brakes off. The two fractions were transferred into separate tubes, washed, and centrifuged for 10 mins at 200 g with the brakes on to remove platelets. The supernatant was removed and pellets were lysed with 1× red blood cell (RBC) lysis buffer (Thermo Fisher) for 5 mins, after which lysis was stopped by the addition PBS containing FBS and EDTA. Samples were then centrifuged at 300 g for 5 mins and the white cell pellet obtained.

### Preparation of Mouse Immune Cells for Lipidomic Analysis

Mouse immune cells were obtained from the peripheral blood of 8-10-week-old, male C57Bl/6J mice. Mice were housed at the AMREP Animal Services Facility with all procedures approved by the institutional animal ethics committee (ARA AEC). Mice were sacrificed via CO₂ asphyxiation and blood obtained via and cardiac puncture. Blood samples were lysed for 15 mins in RBC, after which lysis was stopped with the addition of 1×FACS buffer (HBSS w/o Ca²⁺ and Mg²⁺ containing BSA and 0.5 mM EDTA). Samples were then centrifuged at 3000 rpm for 5 mins at 4° C. and the white cell pellet obtained.

### Antibody Staining and Fluorescence-Activated Cell Sorting (FACS)

Cells were stained with the antibody cocktails outlined in the tables below (Table 1 and 2) for cell-specific surface markers and incubated for 30 minutes on ice. Antibodies were used at a 1:400 dilution unless stated otherwise. Staining was stopped with FACS buffer and cells were subsequently washed and filtered through a 35 μm strainer prior to sorting. The antibodies and sorting panels used to purify the specific human immune cell populations were as follows: Panel 1-B cells, NK cells, CD4 and CD8 T cells; Panel 2-Ly6C^{lo} and Ly6C^{hi} Monocytes, Neutrophils, and Eosinophils.

TABLE 1
Antibodies and sorting panels used to purify human immune cells.

| Cell type | Antibodies used in sorting panels |
|---|---|
| Naïve B cell | L/D CD19⁺ CD27⁺ Mitotracker⁻ |
| Memory B cell | L/D CD19⁺ CD27⁺ Mitotracker⁺ |
| CD4 Naïve T cell | L/D CD3⁺ CD4⁺ CCR7⁺ CD45RA⁺ |
| CD4 Central Memory T cell | L/D CD3⁺ CD4⁺ CCR7⁺ CD45RA⁻ |
| CD4 Effector Memory T cell | L/D CD3⁺ CD4⁺ CCR7⁻ CD45RA⁻ |
| CD8 Naïve T cell | L/D CD3⁺ CD8⁺ CCR7⁺ CD45RA⁺ |
| CD8 Central Memory T cell | L/D CD3⁺ CD8⁺ CCR7⁺ CD45RA⁻ |
| CD8 Effector Memory T cell | L/D CD3⁺ CD8⁺ CCR7⁻ CD45RA⁻ |
| CD56 Dim NK cell | L/D CD3⁻ CD56⁺ CD16⁻ |
| CD56 Bright NK cell | L/D CD3⁻ CD56⁺ CD16⁺ |
| Classical Monocyte | L/D Lin⁻ HLA-DR⁺ CD14⁺ CD16⁻ |
| Intermediate Monocyte | L/D Lin⁻ HLA-DR⁺ CD14⁺ CD16⁺ |
| Non-classical Monocyte | L/D Lin⁻ HLA-DR⁺ CD14^{dim} CD16⁺ |
| Basophil | L/D HLA-DR⁻ CD203c⁺ CD123⁺ |
| Eosinophil | L/D SSC^{high} Siglec8⁺ |
| Neutrophil | L/D SSC^{high} CD15⁺ CD16⁺ |

TABLE 2
Antibodies and sorting panels used to purify murine immune cells.

| Cell type | Antibodies used in sorting panels |
|---|---|
| B cells | L/D CD45⁺ CD3⁻ CD19⁺ |
| NK cells | L/D CD45⁺ CD3⁻ NK1.1⁺ |
| CD4 T cells | L/D CD45⁺ CD3⁺ CD4⁺ |
| CD8 T cells | L/D CD45⁺ CD3⁺ CD8⁺ |
| Ly6C^{lo} monocytes | L/D CD45⁺ CD115⁺ Gr1⁻ |
| Ly6C^{hi} monocytes | L/D CD45⁺ CD115⁺ Gr1⁺ |
| Neutrophils | L/D CD45⁺ CD115⁻ Gr1⁺ |
| Eosinophils | L/D CD45⁺ SSC^{high} SiglecF⁺ |

FACS was performed at the Alfred Medical and Research Education Precinct (AMREP) Flow cytometry core facility. Individual cell populations were sorted using BD FACSAria, BD FACSAria Fusion and BD Influx (BD Biosciences). All gating strategies were first set up based on forward scatter area vs. side scatter area, forward scatter height vs. forward scatter area (doublet exclusion) and side scatter area vs. viability dye (viable cell isolation). A sorted event threshold was set to 250,000 and 60,000 cells for human and murine samples, respectively, and cells were sorted according to the expression of the specific surface markers detailed in the preceding tables. Following isolation, cells were washed with PBS without Ca2+ and Mg2+ and stored at −80° C.

### Lipid Extraction

Cell samples were lyophilised using either a Savant SpeedVac (Thermo Scientific) or a CoolSafe freeze dryer (ScanVac) prior to extraction and resuspended in 10 μl MilliQ H₂O. Lipids were extracted using a modified single phase Folch extraction method (Weir et al., Journal of Lipid Research, 2013). Briefly, 200 μl chloroform-methanol (2:1) was added to each sample along with an internal standard (ISTD) mixture containing non-physiological lipids. In tandem, blank control samples and plasma QCs were extracted and dispersed evenly throughout the extraction order to ensure optimal assay performance and to monitor variation that may arise from the extraction. Samples were subsequently mixed with a rotary mixer for 10 minutes at 90 rpm, sonicated for 30 mins at room temperature and centrifuged at 13,000 rpm for 10 mins to precipitate proteins from the lipid extracts. Supernatant containing the extracted lipids were transferred to a 96 well plate and evaporated using a Savant SpeedVac. Once dried, extracts were reconstituted in H₂O-saturated butanol and methanol with 10 mM ammonium formate and moved to glass vials and stored until mass spectrometry analysis.

### Liquid Chromatography Tandem Mass Spectrometry (LC-MS/MS)

Lipid extracts were analysed using an Agilent 6490 triple quadrupole (QqQ) mass spectrometer coupled to an Agilent 1290 high performance liquid chromatography (HPLC) system and a ZORBAX eclipse plus C18 column (2.1×100 mm 1.8 μm, Agilent) with thermostat set to 60° C. Mass spectrometry analysis was performed in positive mode with dynamic scheduled MRM; transitions, internal standards, and conditions have been previously reported (Huynh et al., Cell Chemical Biology, 2019). Solvents consisted of solvent A (50% H₂O, 30% acetonitrile, 20% isopropanol with 10 mM ammonium formate) and solvent B (1% H₂O, 9% acetonitrile, 90% isopropanol with 10 mM ammonium formate) and followed a 20-minute gradient as outlined in Table 3.

TABLE 3
Solvent gradient used for separation of lipid species.

| Time | Solvent A | Solvent B | Flow | Max. Pressure |
|---|---|---|---|---|
| (min) | (%) | (%) | (mL/min) | limit (bar) |
| 0.00 | 85.00 | 15.00 | 0.4 | 1000.00 |
| 2.50 | 48.00 | 52.00 | 0.4 | 1000.00 |
| 10.00 | 40.00 | 60.00 | 0.4 | 1000.00 |
| 13.00 | 15.00 | 85.00 | 0.4 | 1000.00 |
| 17.00 | 7.00 | 93.00 | 0.4 | 1000.00 |
| 17.10 | 0.00 | 100.00 | 0.4 | 1000.00 |
| 17.90 | 0.00 | 100.00 | 0.4 | 1000.00 |
| 18.00 | 85.00 | 15.00 | 0.4 | 1000.00 |
| 20.00 | 85.00 | 15.00 | 0.4 | 1000.00 |

### Data Normalisation

Analyte areas were obtained from integrating chromatograms that corresponded to a lipid of interest using the Masshunter Quantitative analysis software (Agilent). Lipid concentrations were determined using the following formula:

$$\{ ( \frac{{Area}_{sample analyte}}{{Area}_{ITSD}} ) - Median ( \frac{{Area}_{Blank + ITSD analyte}}{{Area}_{ITSD}} ) \} * {Concentration}_{ITSD} * {Rf}_{analute}$$

In brief, individual analyte areas were divided by the area of the corresponding internal standards and the median of ITSD containing blank samples was subtracted from each analyte (background subtraction). his value was then multiplied by the ITSD concentration and the individual analyte's response factor (Rf). Any values that were zeroed after background subtraction as a consequence of being less than the median value of all blank+ITSD samples, were replaced with 1/10^{th} of the minimum value for the corresponding analyte. Data was ultimately normalised to pmol/μmol total lipidome where the background subtracted data for an individual lipid was divided by the sum of the total lipidome and multiplied by a factor of 106 for ease of graphical representation.

### Outlier Detection

Outlier detection was performed using several complementary approaches. First, a heatmap of log-transformed lipid proportions was created for each sample group. Samples were hierarchically clustered using complete linkage on Euclidean distance. Each heatmap was visually inspected for apparent outlier samples. Concomitantly, a principal component analysis (PCA) was performed on each sample group, and score plots manually examined to aid outlier identification. Finally, the distance to the origin values (distO i.e., the distance of each sample to the origin in PC space), were calculated as a measure of sample extremeness within each PCA, using as many PCs necessary to capture at least 70% of the total variability in the data. DistOs were also averaged across cell types and donors or biological replicates and manually examined to identify potential issues with either source of variability. Other potential sources of variation such as MS injection order and cell count were also accounted for but did not contribute to outlier detection. Samples with the largest distO values within sample groups were flagged as potential outliers and cross-referenced with heatmaps and score plots. Ultimately, samples that were deemed extreme or obvious outliers were then removed.

### Results and Conclusions

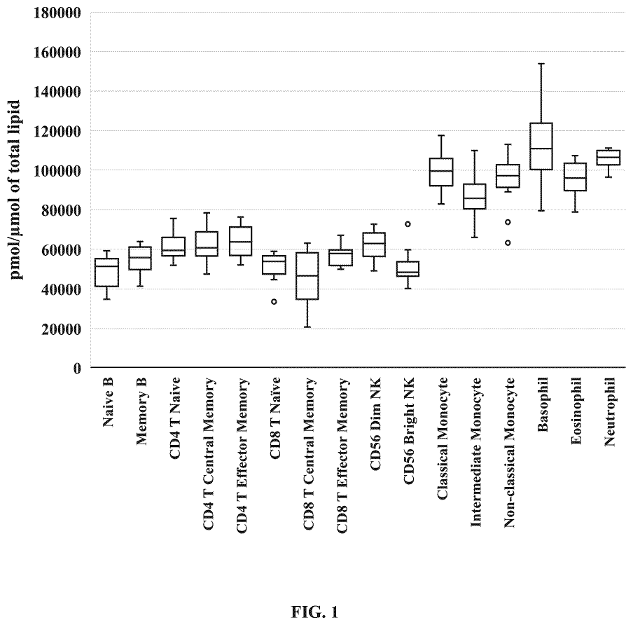

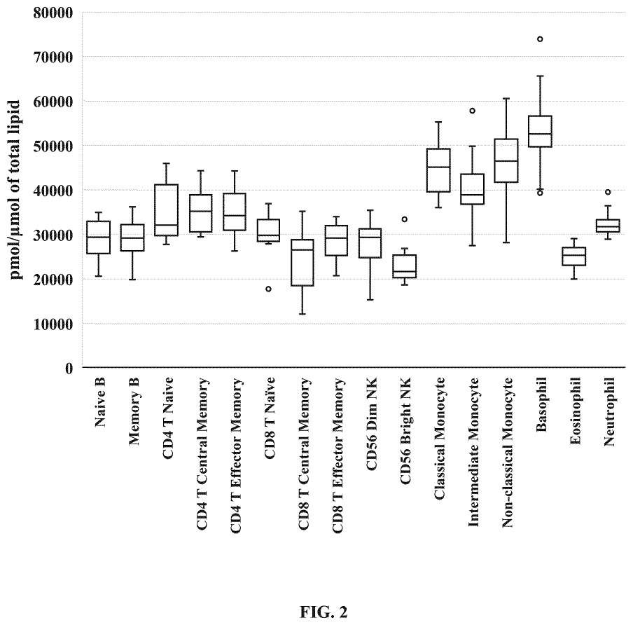

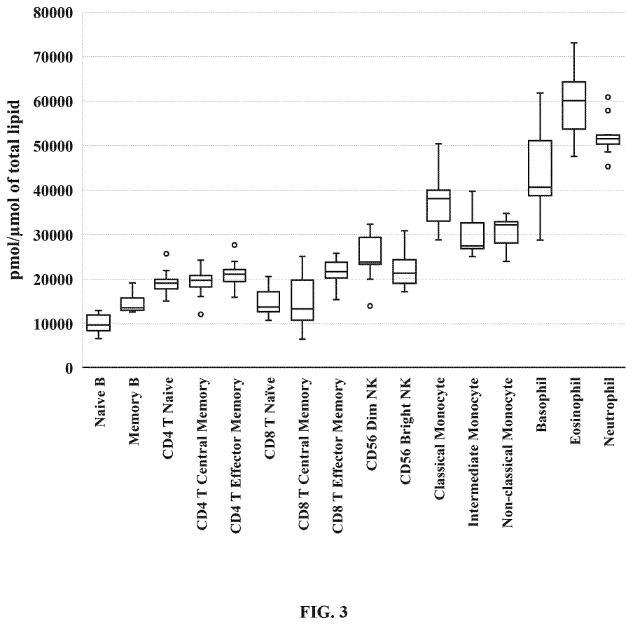

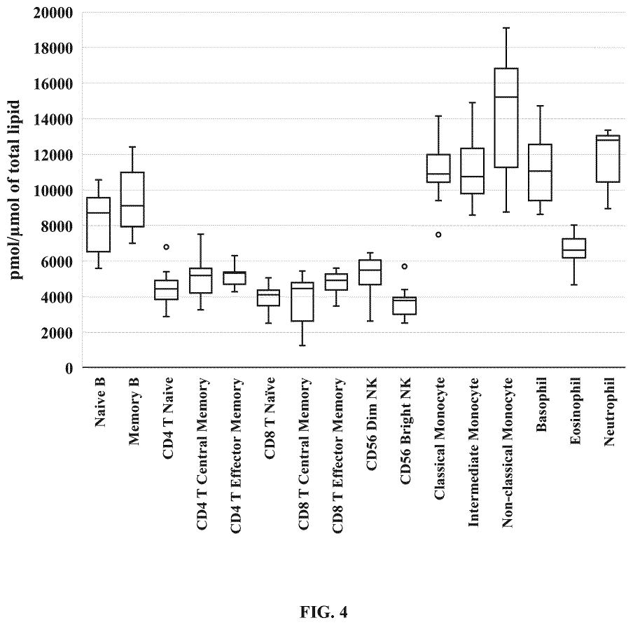

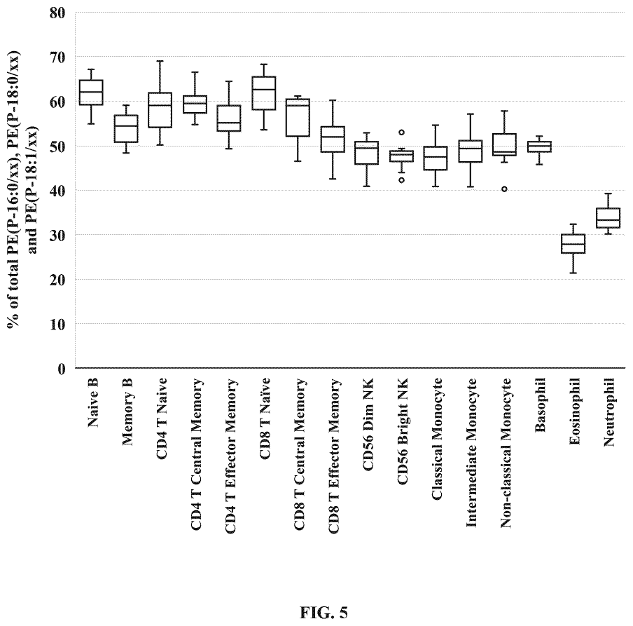

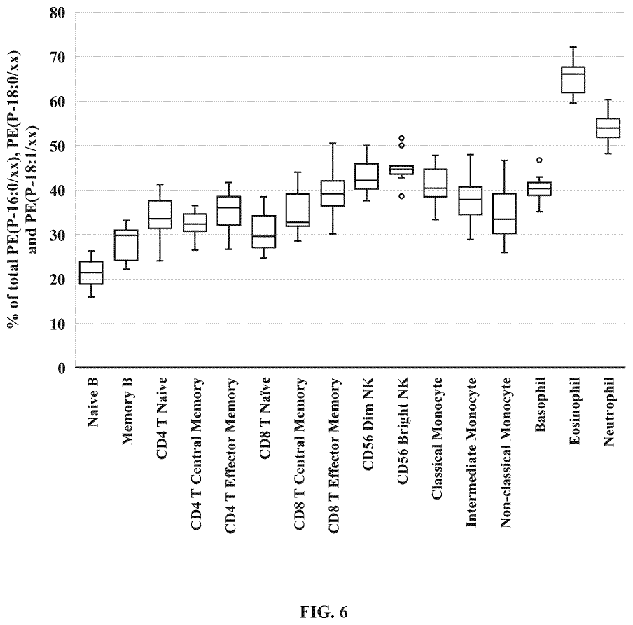

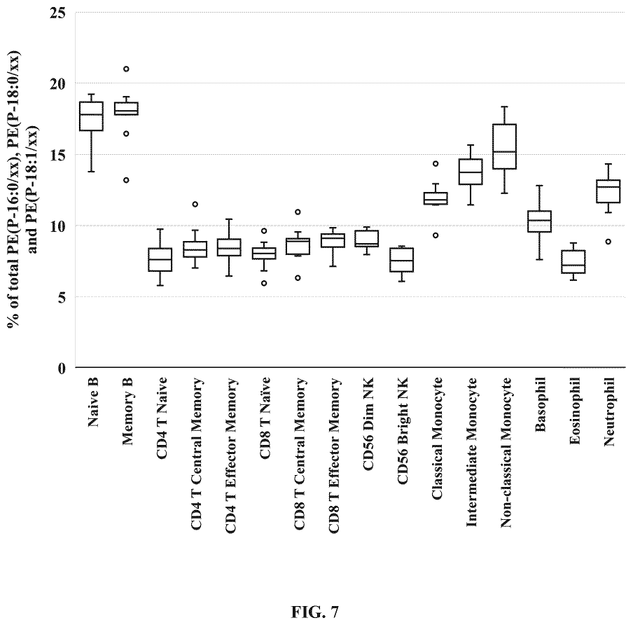

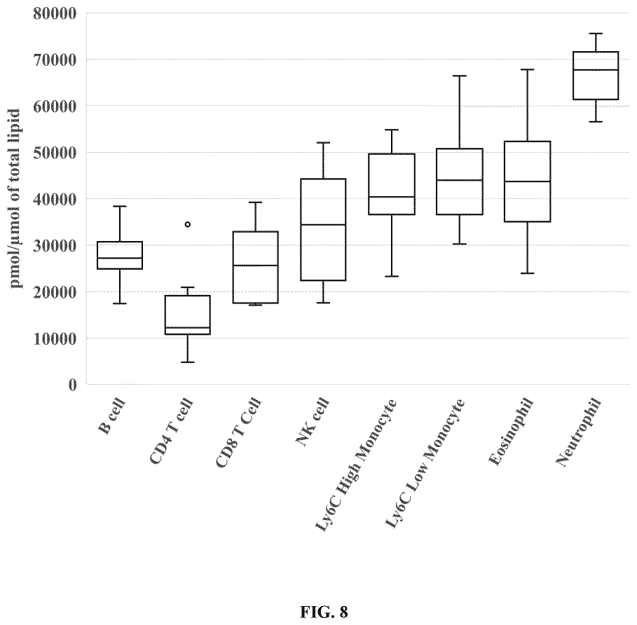

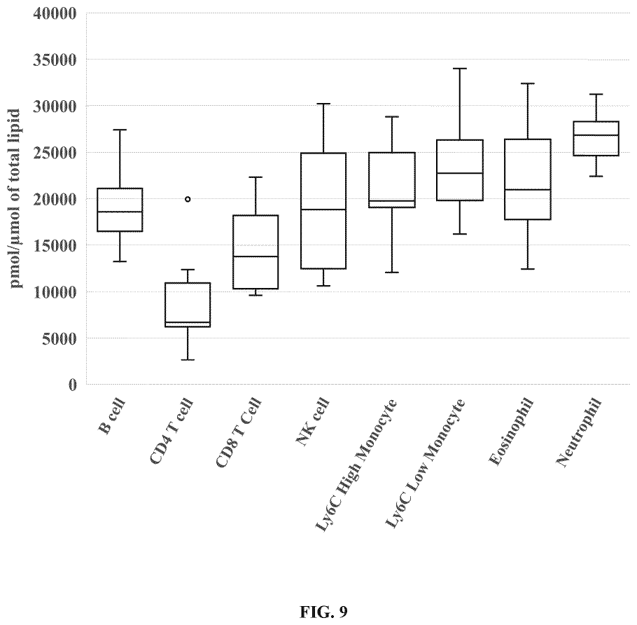

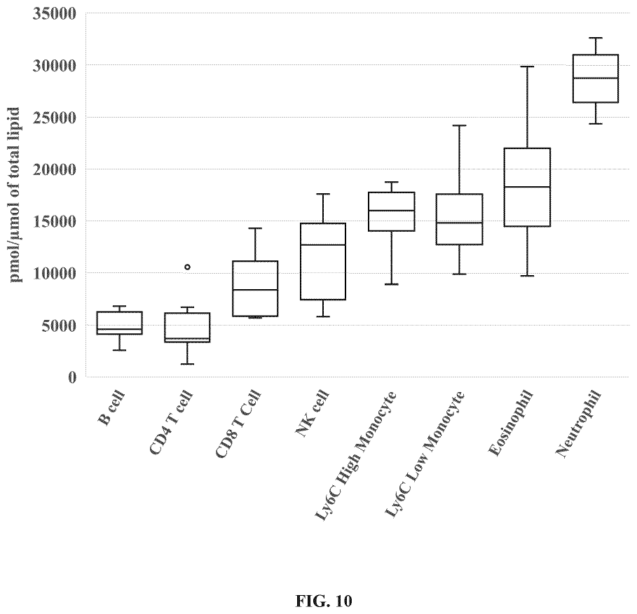

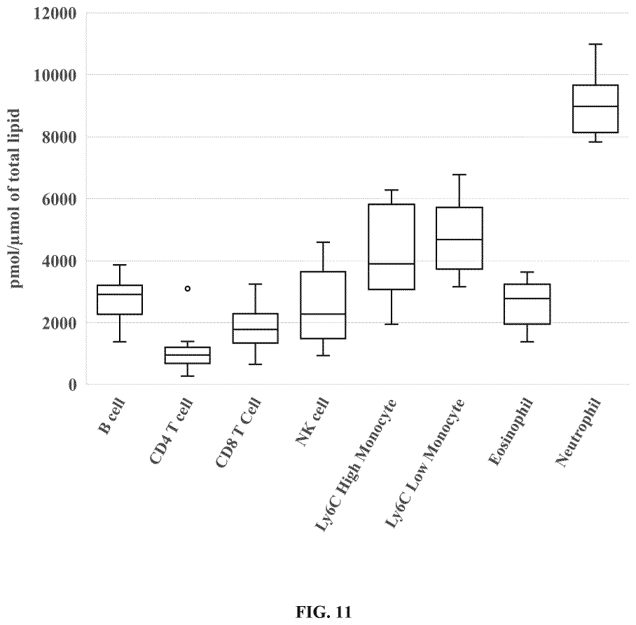

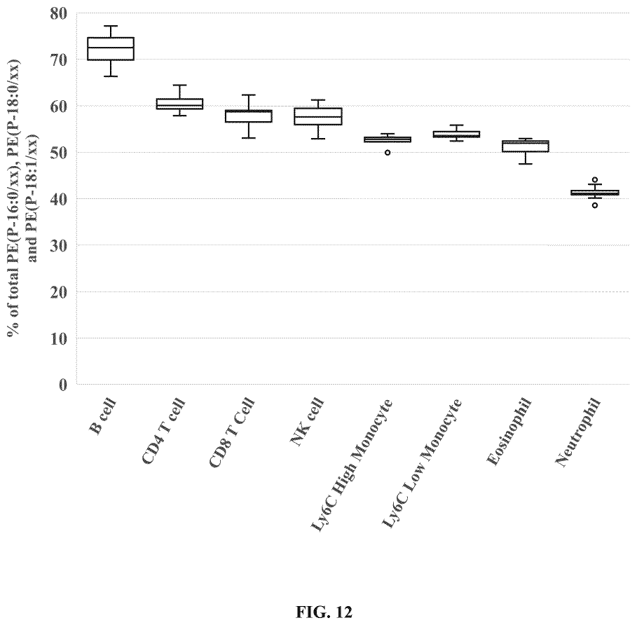

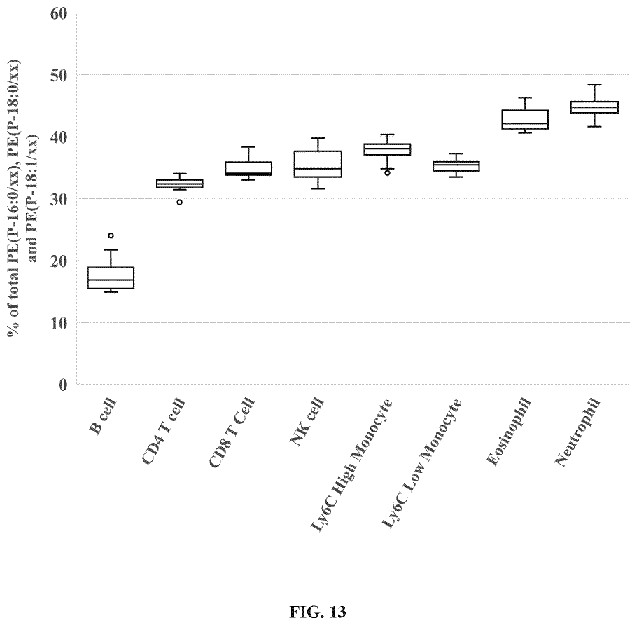

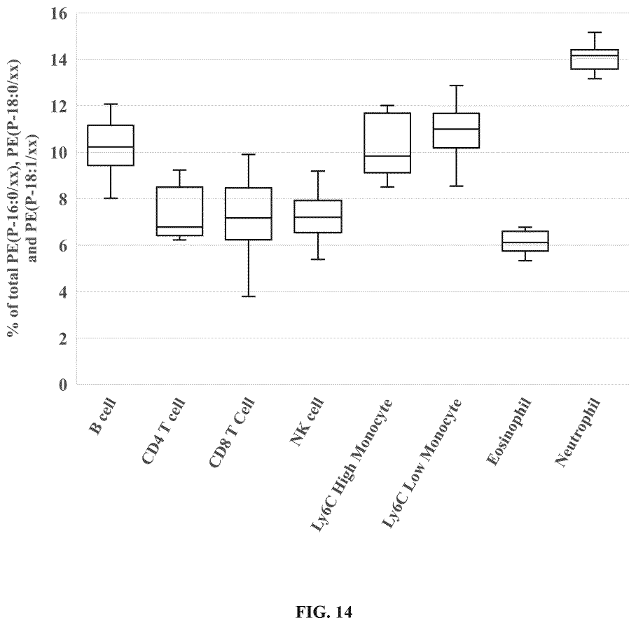

There is a clear distinction in plasmalogen levels between the human lymphoid cells (B cells, T cells and NK cells) and myeloid cells (monocytes, basophils, eosinophils and neutrophils) (FIG. 1 and Table 4). Overall, myeloid cells have a higher level of plasmalogens compared to lymphoid cells. The levels of plasmalogens with different alkenyl chains were also quite different across the different human immune cell types (FIG. 2-4 and Table 4). A unique plasmalogen composition can be provided for all immune cell types, although a more distinctive plasmalogen composition can be provided that are different from and more specific to particular immune cell types including subsets thereof (e.g. T-cells). The proportion of PE(P-16:0/xx) was noticeably lower in the eosinophils and neutrophils than other cell types (FIG. 5 and Table 4). In contrast, the proportion of PE(P-18:0/xx) was noticeably higher in the eosinophils and neutrophils than other cell types (FIG. 6 and Table 4). The proportion of PE(P-18:1/xx) was noticeably higher in the B cells and monocytes than other cell types (FIG. 7 and Table 4). Murine immune cell types also showed a distinct plasmalogen profile (FIG. 8-14 and Table 5). The type of distinction in plasmalogen profile within the murine immune cells was similar to that observed within the human immune cells.

This example demonstrates the diversity in plasmalogen content and composition within immune cell types in both human and murine immune systems and provides ranges for healthy individuals.

### Example 2: Impact of Shark Liver Oil Supplementation on the Circulatory Immune Cell Plasmalogens in Humans

### Study Design

In this double-blind, placebo-controlled crossover study, participants (n=10) were overweight or obese (BMI in the range of 28-40 kg/m²) adult males (aged 25-60 years) with no signs of cardiovascular disease or diabetes. Among the 10 participants, only four fit the definition of having metabolic syndrome according to the strict International Diabetes Federation (IDF) criteria, however, all the participants fulfil at least two criteria of metabolic syndrome. Written informed consent was obtained from all study participants prior to commencement of the study. This study was performed in accordance with the ethical principles set forth in the Declaration of Helsinki and received approval from the Alfred Hospital Ethics Committee (approval number: 436/15).

Participants were randomised into placebo or treatment arms and received 4 g Alkyrol® (purified SLO; Eurohealth, Ireland) per day or placebo (methylcellulose) for 3 weeks followed by a 3-week washout phase and were then crossed over to 3 weeks of the alternate placebo/Alkyrol® treatment. SLO refers to shark liver oil extract comprising a 76:5:19 ratio (molar %) mixture of C18:1 alkenyl glycerolipids, C18:0 alkyl glycerolipids and C16:0 alkyl glycerolipids, respectively. Both Alkyrol® and methylcellulose capsules have similar visual appearance. Participants were instructed to keep their dietary composition and food intake constant during the two treatment phases. Fasting blood samples were collected at the start and end of each intervention.

Isolation of White Blood Cells from Whole Blood

Participants' blood samples were collected in K3-EDTA tubes and centrifuged at (1,711×g, 15 min, room temperature). The top plasma layer was aspirated, and the buffy layer was mixed with 8 ml of phosphate buffered saline (PBS) and layered on top of 5 ml of Ficoll-Paque and centrifuged (400×g, 30 min, room temperature) with the lowest brake. The resulting upper layer (containing plasma and platelets) was discarded, and the thin cloudy layer of white blood cells was collected and transferred to a fresh tube. PBS (8 ml) was added, and the sample was centrifuged (250×g, 10 min, room temperature) with the highest brake. The cells were then resuspended in 1.5 ml of PBS and centrifuged (100×g, 10 min, room temperature). Following centrifugation, the supernatant was discarded, and the white blood cell pellet was suspended in 400 μl PBS and stored at −80° C.

Extraction of Lipids from White Blood Cells

Lipids were extracted using a single phase chloroform:methanol (2:1) extraction protocol (Weir et al., Journal of Lipid Research, 2013). Briefly, 20 μl of white blood cell pellet (suspended in PBS) was combined with 20 volumes (400 μl) of chloroform:methanol (2:1) and 10 μl of internal standard mix and then vortexed. Samples were mixed in a rotary mixer for 10 min, sonicated for 30 min and then allowed to stand for 20 min at room temperature. Samples were then centrifuged (16,000×g, 10 min, 20° C.) and the supernatant was dried under a stream of nitrogen at 40° C. The extracted lipids were finally resuspended with 50 μl of H₂O saturated butanol and 50 μl of methanol containing 10 mM ammonium formate.

Liquid Chromatography Coupled with Mass Spectrometry (LC/MS/MS)

Analysis of lipids were performed on an Agilent 1200 HPLC system coupled to an AB Sciex Q/TRAP 4000 triple quadrupole mass spectrometer using scheduled Multiple Reaction Monitoring (MRM) experiments described previously (Huynh et al., Cell Chemical Biology, 2019). Liquid chromatography separation was performed on a 2.1×100 mm C18 Poroshell column (Agilent, USA) at 400 μl/min. The following gradient conditions were used: 10% B to 55% B over 3 min, then to 70% B over 8 min, to 89% B over 0.1 min, and finally to 100% B over 3.3 min. The solvent was then held at 100% B for 1 min. Equilibration was as follows, solvent was decreased from 100% B to 10% B over 0.1 min and held for an additional 4.5 min. The solvent system consisted of solvent A: 50% H₂O/30% acetonitrile/20% isopropanol (v/v/v) containing 10 mM ammonium formate and solvent B: 1% H₂O/9% acetonitrile/90% isopropanol (v/v/v) containing 10 mM ammonium formate.

The concentrations of individual lipid species were calculated by taking a ratio of the area under the curve of the lipid of interest to the area under the curve of the internal standard of the corresponding lipid class, then multiplying said ratio by the amount of internal standard added into the sample. Response factors were also applied for some lipid species to better estimate true lipid concentrations. Lipid class concentrations were calculated from the sum of individual species within that class.

### Results and Conclusions

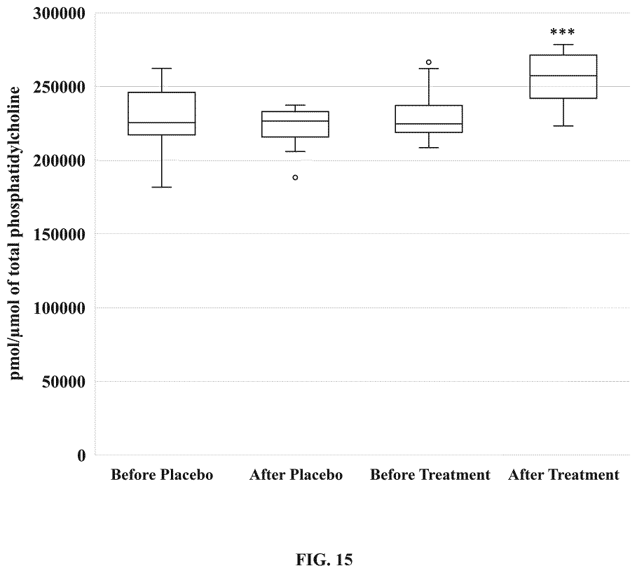

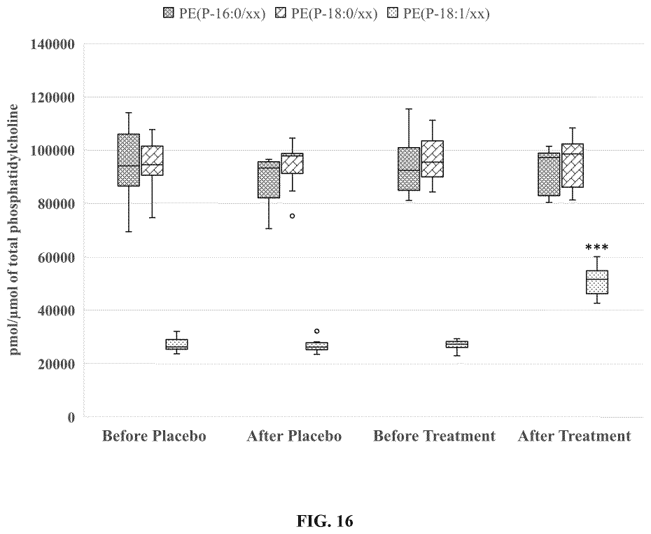

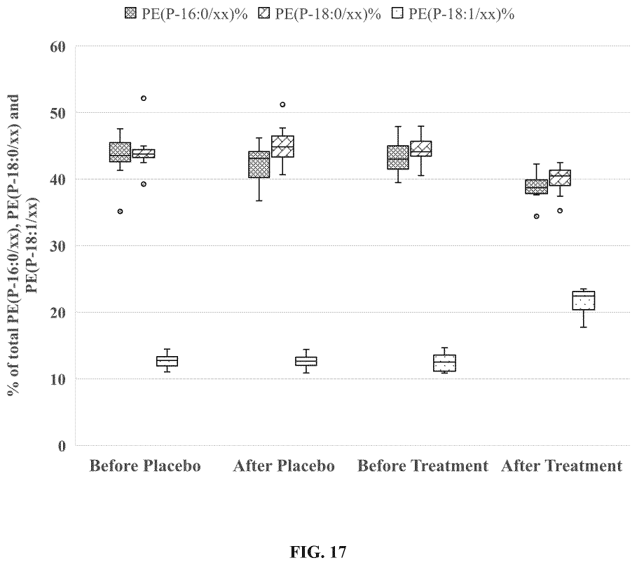

Shark liver oil supplementation significantly increased the level of PE plasmalogens in the circulatory immune cells (FIG. 15 and Table 6). However, this enrichment was mostly confined to the PE(P-18:1/xx) species (FIG. 16 and Table 6). This selective enrichment eventually altered the PE(P) composition of the circulatory immune cells, i.e., increased the proportion of PE(P-18:1/xx) and decreased the proportions of PE(P-16:0/xx) and PE(P-18:0/xx) (FIG. 17 and Table 6). This alteration is most likely due to the alkylglycerol composition of shark liver oil. Almost two thirds of the shark liver alkylglycerols contain O-18:1 alkyl chain (Paul et al, Journal of Lipid Research, 2021).

This example demonstrates that supplementation of humans with plasmalogen precursors can increase the plasmalogen content of immune cells and that the alkyl chain composition of plasmalogen precursors can significantly alter the endogenous plasmalogen composition following supplementation.

### Example 3: Effect of Alkylglycerol Mix Administration on Circulatory Immune Cell Lipidome in Mice

### Methods

A supplementation study was conducted in which mice were fed different supplementation diets over 4 weeks, with lipids quantified in different organs. In this example, eight-week-old male C57BL/6NJ mice housed at 6 mice per cage at 22±1° C. on a 12:12 h light/dark cycle were provided with ad libitum access to either a standard chow diet supplemented with or without an alkylglycerol mix (0.75% of diet w/w) for 4 weeks (n-8-9 per diet group). The alkylglycerol mix was comprised of 50% chimyl alcohol (O-16:0 alkylglycerol (weight %); Nikko Chemicals, Tokyo, Japan), 30% batyl alcohol (O-18:0 alkylglycerol (weight %); abcr GmbH, Karlsruhe, Germany) and 20% selachyl alcohol (O-18:1 alkylglycerol (weight %);′ Astral Scientific, Sydney, NSW, Australia). After 4 weeks of supplementation, mice were euthanised and blood was collected. Immune cells were collected from the whole blood following lysis of red blood cells. The different PE plasmalogen species of the immune cells were then analysed by targeted lipidomics as described in the Example 2.

### Results and Conclusions

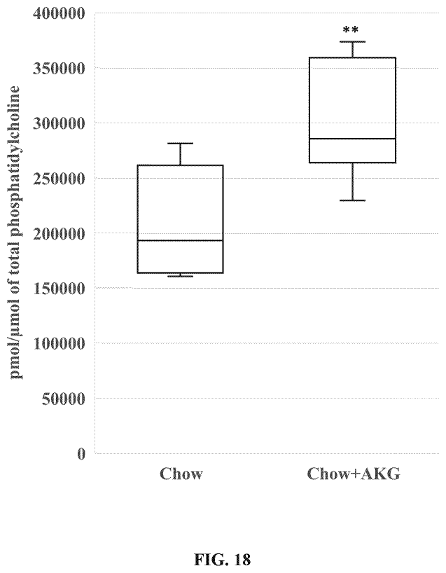

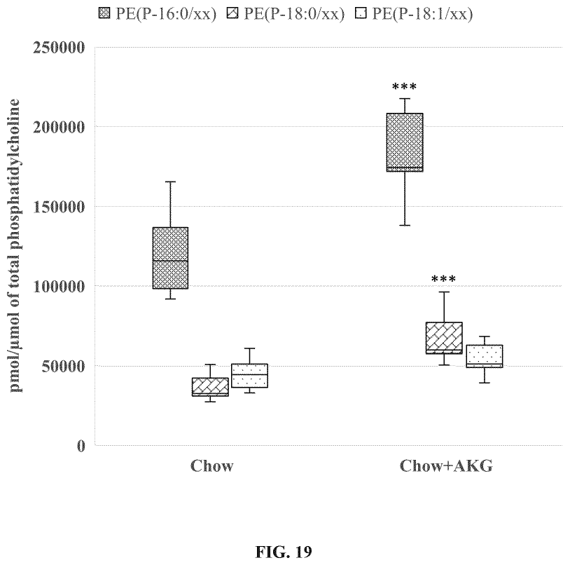

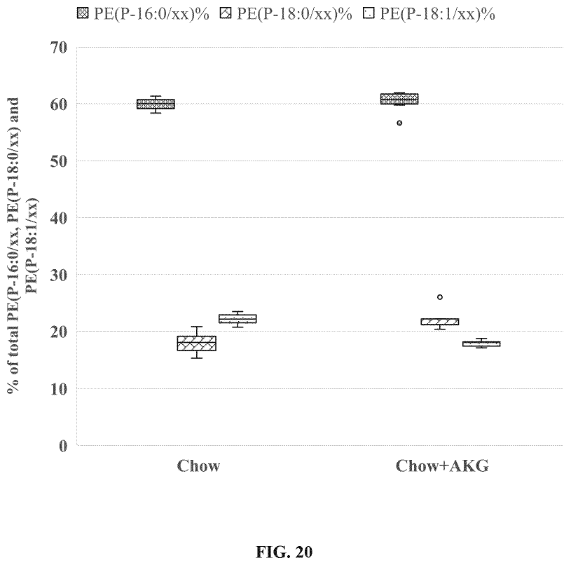

Alkylglycerol mix supplementation significantly increased the level of PE plasmalogens (FIG. 18 and Table 7) in the circulatory immune cells of mice. The supplementation increased the levels of plasmalogen species with different alkenyl chains (FIG. 19 and Table 7) and thereby maintained the endogenous composition of PE(P-16:0/xx), PE(P-18:0/xx) and PE(P-18:1/xx) in the enriched plasmalogen pool (FIG. 20 and Table 7). This demonstrates that a specific composition and/or formulation of the plasmalogen precursor can increase the plasmalogen content of the immune cells while maintaining the composition of the different plasmalogen alkenyl chains.

### Example 4: Modulation of Plasmalogen Content in Immune Cells Protects Against Ferroptosis. Methods

Whole bone marrow cells were collected from the study described in Example 3 and were treated with different concentrations of ML210 (0-10 μM), a GPx4 inhibitor that induces lipid peroxidation and ferroptosis, for 24 hours in a RPMI culture medium with 5% heat inactivated FBS. After treatment, lipid peroxidation and cell viability of T cells were assessed using flow cytometry.

### Cell Viability Analysis

The viability of the cultured cells was determined by DAPI (4′,6-Diamidino-2-Phenlindole, Dihydrochloride; BD Pharmingen), a nucleic acid stain that is excluded from viable cells. Immediately prior to flow cytometry analysis, cells were resuspended in FACS buffer containing 0.05 μg/mL DAPI solution and incubated for 5 mins at room temperature in the dark. After incubation, cells were immediately analysed on the flow cytometer. Upon identification of cell type, cell viability was determined using DAPI fluorescence where DAPI-cells were considered as viable cells and DAPI⁺ cells were considered to be dead cells.

### Lipid Peroxidation Analysis

Lipid peroxidation was assessed using the fluorescent probes C₁₁-BODIPY^{481/591} (4,4-difluoro-5-(4-phenyl-1,3-butadienyl)-4-bora-3a,4a-diaza-s-indacene-3-undecanoic acid; Thermofisher Scientific). C₁₁-BODIPY^{481/591} is a lipid probe that oxidises in the presence of ROS in cell membranes. Upon oxidation of the polyunsaturated butandineyl component, the dye fluoresces with an emission of ~510 nm (FITC channel). For staining, 2 μM of C₁₁-BODIPY^{481/591} was added in the culture medium of cells throughout the incubation period. After treatment, cells were washed, and the fluorescence intensity examined by flow cytometry.

### Results and Conclusions

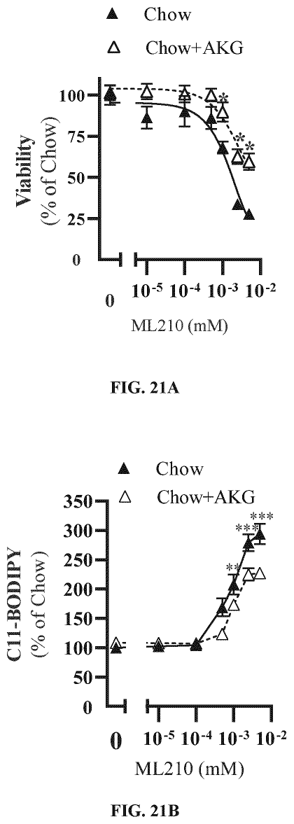

T cells from alkylglycerol mix supplemented mice were significantly protected against oxidative cell death (FIG. 21A) and upon treatment with ML210 accumulated lower levels of lipid peroxides (FIG. 21B). AKG feeding was shown to mitigate cell death and phospholipid peroxidation induced by the GPX4 inhibitor.

This demonstrates the alkylglycerol supplementation can protect T-cells against ferroptotic cell death.

### Example 5: Modulation of Plasmalogen Content in Immune Cells Protects Against Inflammation

### Methods

Following CO₂ asphyxiation of C57B16/J mice, bone marrow (BM) cells were harvested from hind limb bones by flushing bones with RPMI media. Following an initial incubation overnight at a density of 1,000,000 cells/mL in RPMI+Glutamax, 20% L929-cell conditioned media (v/v), 15% foetal bovine serum (v/v; FBS) and 1% penicillin/streptomycin (v/v) (L-cell conditioned media; LCM), non-adherent BM cells were plated into six-well plates (~1,000,000 cells/well). After 3 days, the volume of media in the well was doubled by adding fresh LCM. Cells were used as bone marrow derived macrophages (BMDM) following 7 days of differentiation in LCM. On day 7, the media in the well was aspirated and replaced with media containing RPMI+Glutamax, 5% FBS and 2% BSA (w/v). The BMDMs were then treated with 20 μM of an alkylglycerol mix (55% chimyl alcohol, 35% batyl alcohol and 10% selachyl alcohol) for 48 hours. The alkylglycerol mix was comprised of 55% chimyl alcohol (O-16:0 alkylglycerol (molar %); Bachem, Bubendorf, Switzerland), 35% batyl alcohol (O-18:0 alkylglycerol (molar %); Bachem, Bubendorf, Switzerland) and 10% selachyl alcohol (O-18:1 alkylglycerol (molar %); Astral Scientific, Sydney, NSW, Australia). After 48 hours of treatment, the alkylglycerol containing media was replaced with a lipopolysaccharide (LPS) (1 ug/ml) containing media. After 15 minutes of incubation with LPS, the extent of toll-like receptor (TLR4) activation was assessed. TLR4 activation leads to an inflammatory response in cells and is critically dependent on dimerization. In addition, the level of an inflammatory cytokine [tumour necrosis factor alpha (TNFα)] was measured in cell culture supernatants.

### Assessment of TLR4 Dimerization

BMDMs were washed twice with cold PBS and detached with Accutase (A6964, Sigma-Aldrich). Cells were washed in FACS buffer and BMDM subsequently stained with an antibody that assesses TLR4 dimerization (TLR4-PE-Cy7, clone MTS510, BioLegend) for 30 min on ice. Stained cells were washed with 1 ml FACS buffer, resus-pended in 150 ml of FACS buffer, and surface staining analysed with a BD Fortessa. The geometric mean fluorescence intensity of TLR-PE/Cy7 was recorded and the percentage dimerization of TLR4 calculated.

### Enzyme-Linked Immunosorbent Assay for TNFα Measurement

Levels of TNFα in cell culture supernatants were assessed by an ELISA kit (Catalogue number: 88-7324-22, Thermo Fisher Scientific, USA) according to the manufacturer's instructions.

### Results and Conclusions

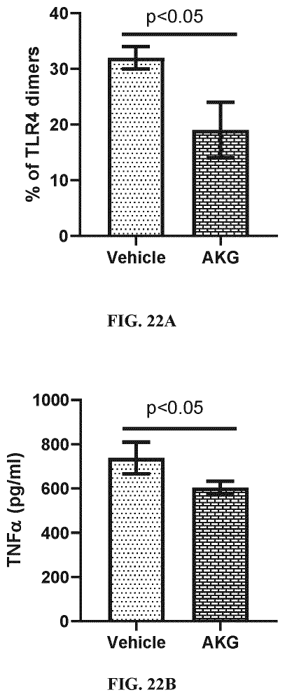

Alkylglycerol mix supplementation in BMDMs suppressed the lipopolysaccharide induced activation of the inflammatory response as measured by the toll-like receptor 4 (TLR4) dimerization (FIG. 22A) and release of an inflammatory cytokine, tumour necrosis factor (TNFα) from the cells (FIG. 22B). This example demonstrates the ant-inflammatory effects of alkylglycerol supplementation.

### Example 6: Alkylglycerol Supplementation Study in Improving the Immune Response to Vaccination and Protecting Against Viral Infection

Plasmalogens are a unique class of membrane glycerophospholipids and are ubiquitous in mammalian tissues. These specialised lipids are important endogenous anti-oxidants with additional roles in regulating membrane fluidity and dynamics, intracellular signalling, immunomodulation, and cholesterol metabolism.

In Example 1, we demonstrated that plasmalogens are particularly enriched in myeloid cells (monocytes, neutrophils, eosinophils and basophils) relative to lymphoid cells (B cells, T cells and NK cells) (FIG. 1). Interestingly, a specialised subset of T cells, follicular helper T (T_{FH}) cells are highly susceptible to ferroptotic cell death (Yao et al, Nature Immunology, 2021). These cells are associated with protective humoral immunity elicited by vaccination.

In Example 3, we demonstrated that supplementation of alkylglycerols can increase plasmalogens within immune cells and in Example 4 we demonstrated that alkylglycerol supplementation can suppress lipid peroxidation and ferroptotic cell death in murine T cells. Based on these observations, we believe that alkylglycerol supplementation can increase the plasmalogen level within T_{FH} cells, and hence lower the level of lipid peroxidation and thereby suppress the ferroptotic death of T_{FH} cells. We propose this as a potential strategy to improve the humoral immunity and protection against viral infection.

We also propose the following studies in murine models to further support the position that alkylglycerol supplementation can be used in improving the immune response to vaccination and protecting against viral infection.

### Study 1: Assess Alkylglycerol Supplementation on Response to Vaccination.

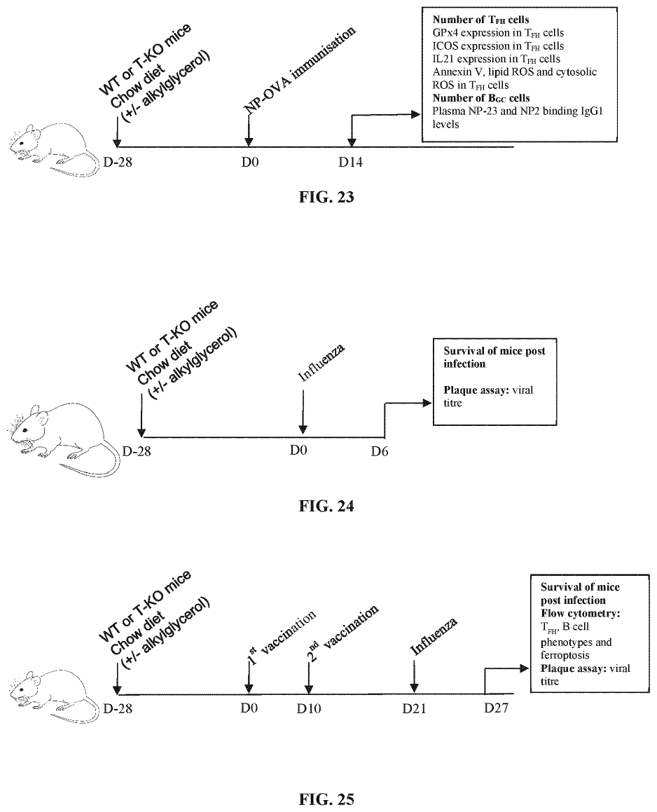

Wild-type mice (n=8 per group) will be placed on chow+/−alkylglycerol diet for four weeks and then will be subcutaneously immunized with a 20 μg NP-OVA (FIG. 23). At day 14 post-immunization, T_{FH} cells, ICOS expression and IL-21 production by T_{FH} cells in popliteal lymph nodes will be analysed. Lipid composition will be analysed by mass spectrometry and cytosolic ROS production and annexin V expression in T_{FH} cells will be analysed by flow cytometry. B_{GC} cells and serum NP23- and NP2-binding IgG1 will also be analysed.

### Study 1A: Assessment of Alkylglycerol Supplementation on Response to Vaccination.

Wild-type (WT) and Cd4-CreGPx4^{flox/flox} (T-KO) mice (n=5-6 per group) were placed on chow+/−alkylglycerol mix (0.75% of diet w/w) diet for four weeks and then were subcutaneously immunized with 20 μg NP-OVA (4-Hydroxy-3-nitrophenylacetyl hapten conjugated to ovalbumin) (FIG. 23). The alkylglycerol mix was comprised of 50% chimyl alcohol (O-16:0 alkylglycerol (weight %); Nikko Chemicals, Tokyo, Japan), 30% batyl alcohol (O-18:0 alkylglycerol (weight %); aber GmbH, Karlsruhe, Germany) and 20% selachyl alcohol (O-18:1 alkylglycerol (weight %); Astral Scientific, Sydney, NSW, Australia). At day 14 post-immunization, number of T_{FH} cells, expression of glutathione peroxidase (Gpx4), inducible T-cell costimulator (ICOS) and interleukin-21 (IL-21) expression in T_{FH} cells of popliteal lymph nodes were analysed. Lipid and cytosolic reactive oxygen species (ROS) production and annexin V expression in T_{FH} cells were analysed by flow cytometry. Number of germinal center B cells (B_{GC}) cells and serum NP23- and NP2-binding IgG1 were also analysed. The analysis of the data is currently underway.

We expect this will provide further support for increasing alkenylphosphatidylethanolamine (PE(P)) in response to alkylglycerol treatment. This will be associated with lower ROS levels and lower cell death as measured by annexin V. This will be associated with an improved immune response indicated by increased B_{GC} cells and serum NP23- and NP2-binding IgG1.

### Study 2: Assess Plasmalogen Supplementation on Response to Viral Infection.

Wild-type mice (WT) and Cd4-CreGPx4^{flox/flox} (T-KO) (n=5-6 per group) will be placed on chow+/−alkylglycerol mix diet for four weeks and then will be intranasally infected with the H1N1 influenza virus at the 50% lethal dose (LD50). At six days post the challenge, influenza-specific antibodies will be analysed by ELISA (H1N1-specific IgG1, IgG2c, IgG2b and IgG3 in serum and bronchoalveolar lavage fluid of virus-challenged mice) (FIG. 24).

We expect this will provide further support for improving survival in mice treated with alkylglycerol relative to control mice and elevated levels of H1N1-specific antibodies in serum and bronchoalveolar lavage fluid of virus-challenged mice treated with alkylglycerol relative to the control mice.

### Study 3: Assess Alkylglycerol Supplementation on the Ability of Vaccination to Protect Against Viral Infection.

Wild-type mice (WT) and Cd4-CreGPx4^{flox/flox} (T-KO) (n=5-6 per group) will be placed on chow+/−alkylglycerol mix diet for 4 weeks and then will be immunised with 100 μl of diluted human flu vaccine containing 1 μg of hemagglutinin derived from the H1N1 influenza virus strain. A booster immunization will be performed 10 days later. Three weeks after the first immunization, mice will be intranasally infected with the H1N1 influenza virus at the 50% lethal dose (LD50). The mice will be compared for survival provided by vaccination-mediated protection. At six days post the challenge, the memory response of T_{FH} cells, B cells and influenza-specific antibodies will be analysed by flow cytometry and ELISA (H1N1-specific IgG1, IgG2c, IgG2b and IgG3 in serum and bronchoalveolar lavage fluid of virus-challenged mice) (FIG. 25).

We expect this will provide further support for improving survival in mice treated with alkylglycerol relative to control mice and an improved H1N1-specific antibody response.

Tables described herein are provided below.

TABLE 4
Plasmalogen content and composition in different human immune cell types.

| | | PE(P- | PE(P- | PE(P- | PE(P- | PE(P- | PE(P- |
|---|---|---|---|---|---|---|---|
| Cell type | PE(P)^{a} | 16:0/xx)^{a} | 18:0/xx)^{a} | 18:1/xx)^{a} | 16:0/xx)%^{b} | 18:0/xx)%^{b} | 18:1/xx)%^{b} |
| Naive B | 51271 | 29318 | 9596 | 8695 | 62 | 21 | 18 |
| | (34632, | (20580, | (6564, | (5579, | (55, 67) | (16, 26) | (14, 19) |
| | 59111) | 34909) | 12871) | 10548) | | | |
| Memory B | 55683 | 29114 | 13456 | 9100 | 54 | 30 | 18 |
| | (41200, | (19787, | (12480, | (6990, | (48, 59) | (22, 33) | (13, 21) |
| | 63797) | 36146) | 19032) | 12403) | | | |
| CD4 T | 60680 | 34525 | 18996 | 4286 | 60 | 33 | 7 |
| Naive | (51796, | (27713, | (14999, | (2870, | (50, 69) | (24, 41) | (6, 10) |
| | 75429) | 45883) | 25590) | 6786) | | | |
| CD4 T | 60655 | 35131 | 19651 | 5179 | 59 | 32 | 8 |
| Central | (47397, | (29398, | (11994, | (3254, | (55, 66) | (26, 36) | (7, 11) |
| Memory | 78261) | 44274) | 24161) | 7502) | | | |
| CD4 T | 63582 | 34166 | 21004 | 5307 | 55 | 36 | 8 |
| Effector | (51925, | (26225, | (15847, | (4266, | (49, 64) | (27, 42) | (6, 10) |
| Memory | 76155) | 44230) | 28112) | 6294) | | | |
| CD8 T | 53810 | 29703 | 13630 | 4101 | 63 | 30 | 8 |
| Naive | (33425, | (17685, | (10632, | (2498, | (54, 68) | (25, 38) | (6, 10) |
| | 58843) | 36883) | 20465) | 5050) | | | |
| CD8 T | 46459 | 26479 | 13222 | 4447 | 59 | 33 | 9 |
| Central | (20586, | (12043, | (6434, | (1244, | (47, 61) | (28, 44) | (6, 11) |
| Memory | 63013) | 35143) | 25008) | 5430) | | | |
| CD8 T | 57728 | 29121 | 21567 | 4909 | 52 | 39 | 9 |
| Effector | (49827, | (20690, | (15291, | (3465, | (42, 60) | (30, 50) | (7, 11) |
| Memory | 66963) | 33942) | 25724) | 6691) | | | |
| CD56 Dim | 62790 | 29278 | 23735 | 5483 | 49 | 42 | 9 |
| NK | (33095, | (15279, | (13861, | (2624, | (41, 53) | (38, 50) | (8, 10) |
| | 72540) | 35386) | 32263) | 6461) | | | |
| CD56 Bright | 48297 | 21613 | 21244 | 3778 | 48 | 45 | 8 |
| NK | (40039, | (18599, | (17059, | (2508, | (42, 53) | (39, 52) | (6, 9) |
| | 72619) | 33357) | 30777) | 5690) | | | |
| Classical | 99415 | 45075 | 37994 | 10884 | 47 | 40 | 12 |
| Monocyte | (82748, | (35981, | (28733, | (7475, | (41, 55) | (33, 48) | (9, 14) |
| | 117337) | 55226) | 50316) | 14136) | | | |
| Intermediate | 85623 | 38820 | 27335 | 10734 | 49 | 38 | 14 |
| Monocyte | (65832, | (27447, | (24974, | (8572, | (41, 57) | (29, 48) | (11, 16) |
| | 109799) | 57732) | 39623) | 14888) | | | |
| Non- | 97021 | 46433 | 32086 | 15196 | 49 | 33 | 15 |
| classical | (63127, | (28122, | (23896, | (8745, | (40, 58) | (26, 47) | (12, 18) |
| Monocyte | 112903) | 60467) | 34673) | 19081) | | | |
| Basophil | 110754 | 52545 | 40580 | 11042 | 50 | 40 | 10 |
| | (79328, | (39270, | (28669, | (8619, | (46, 52) | (35, 47) | (8, 13) |
| | 153716) | 73828) | 61734) | 14710) | | | |
| Eosinophil | 95867 | 25277 | 59991 | 6603 | 28 | 66 | 7 |
| | (78737, | (19914, | (47466, | (4656, | (21, 32) | (59, 72) | (6, 9) |
| | 107207) | 28986) | 72971) | 8014) | | | |
| Neutrophil | 106339 | 31700 | 51438 | 12784 | 33 | 54 | 13 |
| | (96357, | (28858, | (45208, | (8934, | (30, 39) | (48, 60) | (9, 14) |
| | 111003) | 39455) | 60765) | 13340) | | | |

Data are presented as median (minimum, maximum) (n = 9-14 individual donors for each cell type);
^{a}pmol/μmol of total lipid;
^{b}proportion of total PE(16:0/xx), PE(P-18:0/xx) and PE(P-18:1/xx)

TABLE 4b
Specific ratios immune cell subset types calculated from Table 4

| | | | | PE P- | | PE(P- | | PE(P- | |
|---|---|---|---|---|---|---|---|---|---|
| | PE(P- | PE(P- | PE(P- | 16:0/xx)% b | | 18:0/xx)% b | | 18:1/xx)% b | |
| Celltype | 16:0/xx)%^{b} | 18:0/xx)%^{b} | 18:1/xx)%^{b} | low | high | low | high | low | high |
| All immune | 51.1 | 38.3 | 10.8 | 43.9 | 57.0 | 31.4 | 45.9 | 8.4 | 12.9 |
| cells | | | | | | | | | |
| B-cells | 58.0 | 25.5 | 18.0 | 51.5 | 63.0 | 19.0 | 29.5 | 13.5 | 20.0 |
| T-cells | 58.0 | 33.8 | 8.2 | 49.5 | 64.7 | 26.7 | 41.8 | 6.3 | 10.5 |
| NK cells | 48.5 | 43.5 | 8.5 | 41.5 | 53.0 | 38.5 | 51.0 | 7.0 | 9.5 |
| Monocytes | 48.3 | 37.0 | 13.7 | 40.7 | 56.3 | 29.3 | 47.7 | 10.7 | 16.0 |
| Neutrophils | 30.5 | 60.0 | 10.0 | 25.5 | 35.5 | 53.5 | 66.0 | 7.5 | 11.5 |
| Eosiniophils | 30.5 | 60.0 | 10.0 | 25.5 | 35.5 | 53.5 | 66.0 | 7.5 | 11.5 |

TABLE 5
Plasmalogen content and composition in different mouse immune cell types.

| | | | | | PE(P- | PE(P- | PE(P- |
|---|---|---|---|---|---|---|---|
| | | PE(P- | PE(P- | PE(P- | 16:0/xx) | 18:0/xx) | 18:1/xx) |
| Cell type | PE(P)^{a} | 16:0/xx)^{a} | 18:0/xx)^{a} | 18:1/xx)^{a} | %^{b} | %^{b} | %^{b} |
| B Cell | 27152 | 18548 | 4581 | 2904 | 72 | 17 | 10 |
| | (17373, | (13212, | (2553, | (1371, | (66, 77) | (15, 24) | (8, 12) |
| | 38313) | 27384) | 6800) | 3854) | | | |
| CD4 T Cell | 12180 | 6679 | 3682 | 949 | 60 | 32 | 7 |
| | (4699, | (2619, | (1196, | (254, | (58, 64) | (29, 34) | (6, 9) |
| | 34401) | 19903) | 10524) | 3091) | | | |
| CD8 T Cell | 25560 | 13745 | 8372 | 1772 | 59 | 34 | 7 |
| | (17036, | (9571, | (5664, | (636, | (53, 62) | (33, 38) | (4, 10) |
| | 39138) | 22286) | 14246) | 3235) | | | |
| NK Cell | 34328 | 18808 | 12656 | 2266 | 58 | 35 | 7 |
| | (17545, | (10597, | (5793, | (932, | (53, 61) | (32, 40) | (5, 9) |
| | 51988) | 30183) | 17544) | 4589) | | | |
| Ly6C High | 40354 | 19732 | 15955 | 3888 | 53 | 38 | 10 |
| Monocyte | (23236, | (12033, | (8879, | (1941, | (50, 54) | (34, 40) | (8, 12) |
| | 54742) | 28779) | 18692) | 6271) | | | |
| Ly6C Low | 43932 | 22704 | 14765 | 4672 | 53 | 35 | 11 |
| Monocyte | (30183, | (16178, | (9849, | (3152, | (52, 56) | (33, 37) | (9, 13) |
| | 66381) | 33976) | 24166) | 6764) | | | |
| Eosinophil | 43648 | 20940 | 18217 | 2767 | 52 | 42 | 6 |
| | (23872, | (12397, | (9670, | (1374, | (47, 53) | (41, 46) | (5, 7) |
| | 67724) | 32360) | 29836) | 3623) | | | |
| Neutrophil | 67637 | 26803 | 28722 | 8971 | 41 | 45 | 14 |
| | (56480, | (22382, | (24331, | (7821, | (39, 44) | (42, 48) | (13, 15) |
| | 75488) | 31209) | 32583) | 10979) | | | |

Data are presented as median (minimum, maximum) (n = 8-10 individual mice for each cell type);
^{a}pmol/μmol of total lipid;
^{b}proportion of total PE(16:0/xx), PE(P-18:0/xx) and PE(P-18:1/xx)

TABLE 6
Effect of shark liver oil supplementation on plasmalogen content
and composition of circulatory white blood cells in humans.

| | | | | | PE(P- | PE(P- | PE(P- |
|---|---|---|---|---|---|---|---|
| | | PE(P- | PE(P- | PE(P- | 16:0/xx) | 18:0/xx) | 18:1/xx) |
| Group | PE(P)a | 16:0/xx)^{a} | 18:0/xx)^{a} | 18:1/xx)^{a} | %^{b} | %^{b} | %^{b} |
| Before | 225269 | 93914 | 94326 | 26052 | 43 | 44 | 13 |
| Placebo | (181449, | (69243, | (74478, | (23504, | (35, 47) | (39, 52) | (11, 14) |
| | 262032) | 113956) | 107654) | 31893) | | | |
| After | 226320 | 93068 | 97631 | 26025 | 43 | 45 | 13 |
| Placebo | (188027, | (70403, | (75155, | (23316, | (37, 46) | (41, 51) | (11, 14) |
| | 237170) | 96339) | 104451) | 31995) | | | |
| Before | 224415 | 92263 | 95320 | 27118 | 43 | 44 | 12 |
| Treatment | (208253, | (80929, | (84153, | (22755, | (39, 48) | (40, 48) | (11, 15) |
| | 266324) | 115375) | 111148) | 32544) | | | |
| After | 257056*** | 97040 | 98357 | 51569*** | 39 | 40 | 22 |
| Treatment | (223035, | (80189, | (81144, | (42452, | (34, 42) | (35, 42) | (18, 23) |
| | 278240) | 101257) | 108265) | 60042) | | | |

Data are presented as median (minimum, maximum) (n = 10/group);
^{a}pmol/μmol of total lipid;
^{b}proportion of total PE(16:0/xx), PE(P-18:0/xx) and PE(P-18:1/xx).
The nominal significance of the treatment effect was determined using Repeated Measures ANOVA;
***indicates p < 0.001.

TABLE 7
Effect of alkylglycerol mix supplementation on plasmalogen content
and composition of circulatory immune cells in mice.

| | | | | | | PE(P- | PE(P- |
|---|---|---|---|---|---|---|---|
| | | PE(P- | PE(P- | PE(P- | PE(P- | 18:0/xx) | 18:1/xx) |
| Group | PE(P)^{a} | 16:0/xx)^{a} | 18:0/xx)^{a} | 18:1/xx)^{a} | 16:0/xx)^{b} | %^{b} | %^{b} |
| Chow | 193136 | 115532 | 32423 | 44224 | 60 | 18 | 22 |
| | (160230, | (91648, | (27248, | (32852, | (58, 61) | (15, 21) | (21, 23) |
| | 281083) | 165208) | 50580) | 60533) | | | |
| Chow + | 285395*** | 174107*** | 59571*** | 50991 | 61 | 21 | 18 |
| AKG | (229496, | (137834, | (50287, | (39121, | (57, 62) | (20, 26) | (17, 19) |
| | 373349) | 217330) | 96066) | 68015) | | | |

Data are presented as median (minimum, maximum) (n = 8-9 mice/group);
^{a}pmol/μmol of total lipid;
^{b}proportion of total PE(16:0/xx), PE(P-18:0/xx) and PE(P-18:1/xx).
The mean difference between the groups were evaluated by student t-test;
**indicates p < 0.01 and
***indicates p < 0.001.

1. A composition for increasing in vivo ether lipids in immune cells comprising a mixture of two or more ether lipid molecules of Compound 1: 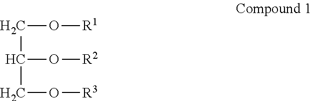
wherein
R¹ is an alkyl or alkenyl group;
R² is hydrogen or 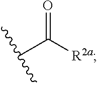
R³ is hydrogen, 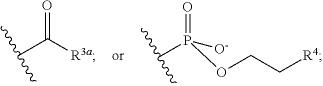
R^{2a} and R^{3a} are each an alkyl or alkenyl group; and
R⁴ is —N(Me)₃⁺ or —NH₃⁺.

2. The composition according to claim 1, wherein the immune cells are selected from B-cells, T-cells, NK cells, monocytes, eosiniophils, or neutrophils.

3. The composition according to claim 1 or claim 2, wherein the composition is for increasing in vivo ether lipids in immune cells at coordinated ratios associated with a non-disease state of the immune cells.

4. The composition according to any one of claims 1 to 3, wherein the increase of ether lipids in the immune cells (in %) is at least about 5, 10, 15, 20, 25, 30, 35, 40, 45, 50, 60, 70, 80, 90, 100, 110, 120, 130, 140, 150, 160, 170, 180, 190, or 200.

5. The composition according to any one of claims 1 to 4, wherein the ether lipids increased in the immune cells are plasmanyl- and/or plasmenyl-phospholipids.

6. The composition according to any one of claims 1 to 5, wherein the ether lipids increased in the immune cells are plasmalogens.

7. The composition according to any one of claims 1 to 6, wherein the ether lipids increased in the immune cells comprise at least two ether lipids of Compound 1 wherein R¹ is selected from C₁₆alkyl, C₁₆alkenyl, C₁₈alkyl, and C₁₈alkenyl.

8. The composition according to any one of claims 1 to 7, wherein the composition comprises at least two ether lipid molecules of Compound 1 wherein R¹ is selected from a C₁₆alkyl, C₁₆alkenyl, C₁₈alkyl, and C₁₈alkenyl.

9. The composition according to any one of claims 1 to 8, wherein the composition comprises at least two ether lipid molecules of Compound 1 wherein R¹ is selected from 16:0 alkyl, 18:0 alkyl, and 18:1 alkenyl.

10. The composition according to any one of claims 1 to 9, wherein the composition comprises an ether lipid molecule of Compound 1 wherein R¹ is 16:0 alkyl and an ether lipid molecule of Compound 1 wherein R¹ is 18:0 alkyl.

11. The composition according to claim 10, wherein the ratio of the 16:0 alkyl ether lipid molecules to the 18:0 alkyl ether lipid molecules is in a range of about 0.85:1 to 3:1, 1:1 to 2.5:1, or 1.5:1 to 2:1.

12. The composition according to claim 10, wherein the composition has a molar percent of 16:0 alkyl ether lipid molecules in the range of from 35% to 80%, 40% to 70%, or 45% 65%, and a molar percent of 18:0 alkyl ether lipid molecules in the range of from 15% to 60%, 20% to 50%, or 25% to 45%.

13. The composition according to any one of claims 1 to 12, wherein the composition comprises an ether lipid molecule of Compound 1 wherein R¹ is 16:0 alkyl and an ether lipid molecule of Compound 1 wherein R¹ is 18:1 alkenyl.

14. The composition according to claim 13, wherein the ratio of the 16:0 alkyl ether lipid molecule to the 18:1 alkenyl ether lipid molecule is in a range of about 2:1 to 10:1, 3:1 to 7:1, or 4:1 to 6:1.

15. The composition according to claim 13, wherein the composition has a molar percent of 16:0 alkyl ether lipid molecules in the range of from 35% to 80%, 40% to 70%, or 45% 65%, and a molar percent of 18:1 alkenyl ether lipid molecules in the range of from about 1% to 20%, 2% to 18%, or 5% to 15%.

16. The composition according to any one of claims 1 to 15, wherein the composition comprises an ether lipid molecule of Compound 1 wherein R¹ is 18:0 alkyl and an ether lipid molecule of Compound 1 wherein R¹ is 18:1 alkenyl.

17. The composition according to claim 16, wherein the ratio of the 18:0 alkyl ether lipid molecule to the 18:1 alkenyl ether lipid molecule is in a range of about 1.5:1 to 10:1, 2:1 to 7:1, or 3:1 to 6:1.

18. The composition according to claim 17, wherein the composition has a molar percent of 18:0 alkyl ether lipid molecules in the range of from 15% to 60%, 20% to 50%, or 25% to 45%, and a molar percent of 18:1 alkenyl ether lipid molecules in the range of from about 1% to 20%, 2% to 18%, or 5% to 15%.

19. The composition according to any one of claims 1 to 18, wherein the composition comprises an ether lipid molecule of Compound 1 wherein R¹ is 16:0 alkyl, an ether lipid molecule of Compound 1 wherein R¹ is 18:0 alkyl, and an ether lipid molecule of Compound 1 wherein R¹ is 18:1 alkenyl.

20. The composition according to claim 19, wherein the composition has a molar percent of 16:0 alkyl ether lipid molecules in the range of from 35% to 80%, 40% to 70%, or 45% 65%, a molar percent of 18:0 alkyl ether lipid molecules in the range of from 15% to 60%, 20% to 50%, or 25% to 45%, and a molar percent of 18:1 alkenyl ether lipid molecules in the range of from about 1% to 20%, 2% to 18%, or 5% to 15%.

21. The composition according to any one of claims 1 to 20, wherein the ether lipids having an 18:1 alkenyl R¹ group, ether lipids having an 18:0 alkyl R¹ group, and/or ether lipids having a 16:0 alkyl R¹ group, if present, together comprise at least about 10, 20, 30, 40, 50, 60, 70, 80, or 90% of total ether lipids in the composition.

22. A composition according to any one of claims 1 to 21, wherein the composition is in the form of a product which is a dietary supplement, capsule, syrup, liquid, food or beverage.

23. The composition according to any one of claims 1 to 22, wherein the composition is prepared by mixing a plurality of ether lipids in ratios and/or levels corresponding with ratios and/or levels associated with a non-disease state in vivo immune cell.

24. A composition for increasing in vivo ether lipids in immune cells comprising a mixture of two or more ether lipid molecules of Compound 1A: 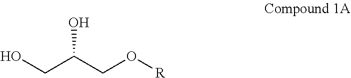
wherein
R is an alkyl or alkenyl group;
wherein the alkyl or alkenyl group is a C₁-C₃₀ alkyl, or C₁-C₃₀ alkenyl chain.

25. The composition according to claim 24, wherein the immune cells are selected from B-cells, T-cells, NK cells, monocytes, eosinophils, or neutrophils.

26. The composition according to claim 25, wherein the immune cells are T-cells.

27. The composition according to claim 25, wherein the immune cells are monocytes.

28. The composition according to any one of claims 24 to 27, wherein the composition is for increasing in vivo ether lipids in immune cells at coordinated ratios associated with a non-disease state of the immune cells.

29. The composition according to any one of claims 24 to 28, wherein the increase of ether lipids in the immune cells (in %) is at least about 5, 10, 15, 20, 25, 30, 35, 40, 45, 50, 60, 70, 80, 90, 100, 110, 120, 130, 140, 150, 160, 170, 180, 190, or 200.

30. The composition according to any one of claims 24 to 29, wherein the ether lipids increased in the immune cells are plasmanyl- and/or plasmenyl-phospholipids.

31. The composition according to any one of claims 24 to 30, wherein the ether lipids increased in the immune cells are plasmalogens.

32. The composition according to any one of claims 24 to 31, wherein the ether lipids increased in the immune cells comprise at least two ether lipids of Compound 1A wherein R is selected from C₁₆alkyl, C₁₆alkenyl, C₁₈alkyl, and C₁₈alkenyl.

33. The composition according to any one of claims 24 to 32, wherein the mixture of two or more ether lipid molecules of Compound 1A comprises two or more ether lipid molecules selected from chimyl alcohol, batyl alcohol and selachyl alcohol.

34. The composition of any one of claims 24 to 33, wherein the mixture of two or more ether lipids comprises chimyl alcohol.

35. The composition of any one of claims 24 to 34, wherein the mixture of two or more ether lipids comprises chimyl alcohol in a molar percent amount of 19-55% of the total ether lipids or comprises chimyl alcohol in a weight percent amount of 19-55% of the total ether lipids.

36. The composition of any one of claims 24 to 35, wherein the mixture of two or more ether lipids comprises batyl alcohol.

37. The composition of any one of claims 24 to 36, wherein the mixture of two or more ether lipids comprises batyl alcohol in a molar percent amount of 5-35% of the total ether lipids or comprises batyl alcohol in a weight percent amount of 5-35% of the total ether lipids.

38. The composition of any one of claims 24 to 37, wherein the mixture of two or more ether lipids comprises selachyl alcohol.

39. The composition of any one of claims 24 to 38, wherein the mixture of two or more ether lipids comprises selachyl alcohol in a molar percent amount of 10-76% of the total ether lipids or comprises selachyl alcohol in a weight percent amount of 10-76% of the total ether lipids.

40. The composition of claim 33, wherein the mixture of two or more ether lipid molecules of Compound 1A comprises a mixture of chimyl alcohol and batyl alcohol.

41. The composition of claim 33 or claim 40, wherein the mixture of two or more ether lipids comprises a mixture of chimyl alcohol in a molar percent amount of 19-55% of the total ether lipids and batyl alcohol in a molar percent amount of 5-35% of the total ether lipids or comprises a mixture of chimyl alcohol in a weight percent amount of 19-55% of the total ether lipids and batyl alcohol in a weight percent amount of 5-35% of the total ether lipids.

42. The composition of claim 33, wherein the mixture of two or more ether lipid molecules of Compound 1A comprises a mixture of chimyl alcohol and selachyl alcohol.

43. The composition of claim 33 or claim 42, wherein the mixture of two or more ether lipids comprises a mixture of chimyl alcohol in a molar percent amount of 19-55% of the total ether lipids and selachyl alcohol in a molar percent amount of 10-76% of the total ether lipids or comprises a mixture of chimyl alcohol in a weight percent amount of 19-55% of the total ether lipids and selachyl alcohol in a weight percent amount of 10-76% of the total ether lipids.

44. The composition of claim 33, wherein the mixture of two or more ether lipid molecules of Compound 1A comprises a mixture of batyl alcohol and selachyl alcohol.

45. The composition of claim 33 or claim 44, wherein the mixture of two or more ether lipids comprises a mixture of batyl alcohol in a molar percent amount of 5-35% of the total ether lipids and selachyl alcohol in a molar percent amount of 10-76% of the total ether lipids or comprises a mixture of batyl alcohol in a weight percent amount of 5-35% of the total ether lipids and selachyl alcohol in a weight percent amount of 10-76% of the total ether lipids.

46. The composition of claim 33, wherein the mixture of two or more ether lipid molecules of Compound 1A comprises chimyl alcohol, batyl alcohol and selachyl alcohol.

47. The composition of claim 46, wherein the chimyl alcohol is present in a molar percent amount of 19% of the total ether lipids, batyl alcohol is present in a molar percent amount of 5% of the total ether lipids, and selachyl alcohol is present in a molar percent amount of 76% of the total ether lipids.

48. The composition of claim 46, wherein the chimyl alcohol is present in an amount of 50% of the total ether lipids by weight, batyl alcohol is present in an amount of 30% of the total ether lipids by weight, and selachyl alcohol is present in an amount of 20% of the total ether lipids by weight.

49. The composition of claim 46, wherein the chimyl alcohol is present in a molar percent amount of 55% of the total ether lipids, batyl alcohol is present in a molar percent amount of 35% of the total ether lipids, and selachyl alcohol is present in a molar percent amount of 10% of the total ether lipids.

50. The composition according to any one of claims 24 to 49, wherein the composition is formulated for oral administration.

51. The composition according to any one of claims 24 to 50, wherein the composition is a tablet, a capsule, a solution, a mouthwash, a suspension, a syrup, a powder, a gum, a food product, a beverage, a dietary supplement, a confectionary, a lozenge, a sublingual delivery system, or a rapid-melt formulation.

52. The composition according any one of claims 24 to 51, wherein the composition is a food product.

53. The composition according any one of claims 24 to 51, wherein the composition is a dietary supplement.

54. The composition according any one of claims 24 to 52, wherein the food product is a medical food product.

55. The composition according any one of claims 24 to 52, wherein the food product is infant formula.

56. The composition according to any one of claims 24 to 55, wherein the composition is prepared by mixing a plurality of ether lipids in ratios and/or levels corresponding with ratios and/or levels associated with a non-disease state in vivo immune cell.

57. A composition of any one of claims 1 to 56 for use in therapy.

58. A nutritional composition comprising a composition of any one of claims 1 to 57 and a potable excipient.

59. A pharmaceutical composition comprising a composition of any one of claims 1 to 57 and a pharmaceutically acceptable excipient.

60. A method of increasing in vivo ether lipids in immune cells comprising administering to a subject an effective amount of a composition according to any one of claims 1 to 59.

61. The method according to claim 60, wherein the immune cells are selected from B-cells, T-cells, NK cells, monocytes, eosinophils, and/or neutrophils.

62. The method according to claim 60 or claim 61, wherein the method is for increasing in vivo ether lipids in immune cells at coordinated ratios associated with a non-disease state of the immune cells.

63. The method according to any one of claims 60 to 62, wherein the increase of ether lipids in the immune cells (in %) of the subject is at least about 5, 10, 15, 20, 25, 30, 35, 40, 45, 50, 60, 70, 80, 90, 100, 110, 120, 130, 140, 150, 160, 170, 180, 190, or 200.

64. The method according to any one of claims 60 to 63, wherein the ether lipids increased in the immune cells are plasmanyl- and/or plasmenyl-phospholipids.

65. The method according to any one of claims 60 to 64, wherein the ether lipids increased in the immune cells are plasmalogens.

66. The method according to any one of claims 60 to 65, wherein the ether lipids increased in the immune cells comprise at least two ether lipids of Compound 1 wherein R¹ is selected from C₁₆alkyl, C₁₆alkenyl, C₁₈alkyl, and C₁₈alkenyl.

67. The method according to any one of claims 60 to 66, wherein the ether lipids increased in the immune cells are in coordinated ratios corresponding to the ether lipid molecules as defined in any one of claims 1 to 56.

68. The method according to any one of claims 60 to 67, further comprising (i) identifying a subject with low amount of plasmalogen content in their immune cells, and (ii) administering to the subject a composition as defined in any one of claims 1 to 56 having coordinated ratios of ether lipid molecules that substantially correspond to the plasmalogen profile in the immune cells to elevate two or more key plasmalogen species in a coordinated manner to increase the level of plasmalogens in the immune cells.

69. The method according to claim 68, wherein the total plasmalogen content in the immune cells of a subject prior to treatment is less than about 10, 9, 8, 7, 6, 5, 4, 3, or 2 molar % of total lipids.

70. A method of treating or preventing a disease, disorder, or condition, associated with the immune system in a subject, the method comprising administering an effective amount of a composition according to any one of claims 1 to 59 to the subject.

71. A method of improving immune health or an immune response in a subject, the method comprising administering an effective amount of a composition according to any one of claims 1 to 59 to the subject.

72. The method of claim 70 or claim 71, wherein the disease, disorder, or condition in a subject is cardiovascular disease, Alzheimer's disease, viral infection, metabolic disease, diabetes, cardiovascular disease, obesity, fatty liver disease, an inflammatory condition, or dyslipidaemia.

73. A method of improving response to vaccination in a subject, the method comprising administering an effective amount of a composition according to any one of claims 1 to 59 to the subject.

74. A method of treating or preventing an adverse response to a viral infection in a subject, the method comprising administering an effective amount of a composition according to any one of claims 1 to 59 to the subject to provide an enhanced immune response in the subject.

75. A method of treating or preventing a disease, disorder, or condition associated with ferroptosis of immune cells in a subject, the method comprising administering an effective amount of a composition according to any one of claims 1 to 59 to the subject.

76. Use of a composition of any one of claims 1 to 59 for the manufacture of a medicament for improving immune health or an immune response in a subject in need thereof, or for treating or preventing an adverse response to a vaccination and/or viral infection in a subject.

77. Use of a composition of any of claims 1 to 59 for the manufacture of a medicament for treating or preventing a disease, disorder, or condition associated with ferroptosis of immune cells in a subject.

78. A method of reducing ferroptosis in immune cells comprising administering to a subject an effective amount of a composition according to any one of claims 1 to 59.

79. The method of claim 78, wherein the immune cells are selected from B-cells, T-cells, NK cells, monocytes, eosinophils, or neutrophils.

80. The method of claim 79, wherein the immune cells are T-cells.

81. The method of claim 80, wherein the T-cells are follicular helper T (T_{FH}) cells.

82. A method of treating or preventing a disease, disorder, or condition associated with the immune system in a subject, the method comprising administering an effective amount of a composition according to any one of claims 1 to 59 to the subject.

83. The method of claim 82, wherein the disease, disorder, or condition associated with the immune system in a subject is Alzheimer's disease, a viral infection, a metabolic disease, a cardiovascular disease, obesity, fatty liver disease, an inflammatory condition, or dyslipidaemia.

84. The method of claim 82 or claim 83, wherein the disease, disorder, or condition associated with the immune system in a subject is a viral infection, a metabolic disease, or an inflammatory condition.

85. The method of claim 83 or claim 84, wherein the viral infection is COVID-19 infection.

86. The method of claim 83 or claim 84, wherein the metabolic disease is diabetes.

87. The method of claim 86, wherein the diabetes is type 2 diabetes.

88. The method of claim 83 or claim 84 wherein the inflammatory condition is asthma or atopic dermatitis.

89. The method of any one of claim 83, 84, or 88, wherein the inflammatory condition is associated with an increase in the levels of inflammatory cytokines.

90. The method of claim 89, wherein the increase in the levels of inflammatory cytokines is an increase in the levels of TNFα.

91. The method of claim 75 or claim 77, wherein the treating or preventing a disease, disorder, or condition associated with ferroptosis of immune cells in a subject comprises a reduction in oxidative cell death.

92. The method of claim 91, wherein the treating or preventing a disease, disorder, or condition associated with ferroptosis of immune cells in a subject comprises a reduction in ferroptosis.

93. The method of claim 91 or claim 92, wherein the treating or preventing a disease, disorder, or condition associated with ferroptosis of immune cells in a subject comprises a reduction in the progression and symptoms associated with tumors, inflammatory diseases, bacterial infections, pulmonary fibrosis, hepatitis, inflammatory bowel disease, neurodegenerative diseases, kidney injury, ischemia-reperfusion injury, skeletal muscle injury, neurodegeneration, Alzheimer's disease, and frailty in the elderly.

94. The method of any one of claims 60 to 93, wherein the composition of ether lipids is administered in a dose of from 0.1 mg per day to 4,000 mg per day.

95. The method of any one of claims 60 to 94, wherein the composition of ether lipids is administered in a dose of from 0.1 mg per day to 2,000 mg per day.

96. The method of any one of claims 60 to 95, wherein the composition of ether lipids is administered in a dose of from 25 mg per day to 1600 mg per day.

97. The method of any one of claims 60 to 96, wherein the composition of ether lipids is administered in a dose of 400 mg, 800 mg, or 1600 mg per day.

98. The method of any one of claims 60 to 96, wherein the composition of ether lipids is administered in a dose of 200 mg per day.

99. The method of any one of claims 60 to 96, wherein the composition of ether lipids is administered in a dose of from 25 mg per day to 100 mg per day.
